SEQUENCE LISTING (32 sequences)

SEQ ID NO: 1 - protein, 8 aa, Artificial Sequence
  Description of Artificial Sequence Synthetic peptide
FCYWKTCT

SEQ ID NO: 2 - protein, 8 aa, Artificial Sequence
  Description of Artificial Sequence Synthetic peptide
FCYWKTCT

SEQ ID NO: 3 - protein, 8 aa, Artificial Sequence
  Description of Artificial Sequence Synthetic peptide
FCYWKTCT

SEQ ID NO: 4 - protein, 8 aa, Artificial Sequence
  Description of Artificial Sequence Synthetic peptide
FCYWKTCT

SEQ ID NO: 5 - protein, 4 aa, Artificial Sequence
  Description of Artificial Sequence Synthetic peptide
KYKK

SEQ ID NO: 6 - protein, 5 aa, Artificial Sequence
  Description of Artificial Sequence Synthetic peptide
QAKYK

SEQ ID NO: 7 - protein, 4 aa, Artificial Sequence
  Description of Artificial Sequence Synthetic peptide
AKYK

SEQ ID NO: 8 - protein, 4 aa, Artificial Sequence
  Description of Artificial Sequence Synthetic peptide
AKYK

SEQ ID NO: 9 - protein, 44 aa, Artificial Sequence
  Description of Artificial Sequence Synthetic polypeptide
SHIQIPPGLTELLQGYTVEVLRQQPPDLVEFAVEYFTRLREARA

SEQ ID NO: 10 - protein, 45 aa, Artificial Sequence
  Description of Artificial Sequence Synthetic polypeptide
CGHIQIPPGLTELLQGYTVEVLRQQPPDLVEFAVEYFTRLREARA

SEQ ID NO: 11 - protein, 17 aa, Artificial Sequence
  Description of Artificial Sequence Synthetic peptide
QIEYLAKQIVDNAIQQA

SEQ ID NO: 12 - protein, 21 aa, Artificial Sequence
  Description of Artificial Sequence Synthetic peptide
CGQIEYLAKQIVDNAIQQAGC

SEQ ID NO: 13 - protein, 55 aa, Artificial Sequence
  Description of Artificial Sequence Synthetic polypeptide
GSGGGGSGGGGSHIQIPPGLTELLQGYTVEVLRQQPPDLVEFAVEYFTRLREARA

SEQ ID NO: 14 - protein, 29 aa, Artificial Sequence
  Description of Artificial Sequence Synthetic peptide
GSGGGGSGGGGSQIEYLAKQIVDNAIQQA

SEQ ID NO: 15 - protein, 4 aa, Artificial Sequence
  Description of Artificial Sequence Synthetic peptide
AKYK

SEQ ID NO: 16 - protein, 4 aa, Artificial Sequence
  Description of Artificial Sequence Synthetic peptide
DKYK

SEQ ID NO: 17 - protein, 3 aa, Artificial Sequence
  Description of Artificial Sequence Synthetic peptide
KYK

SEQ ID NO: 18 - protein, 4 aa, Artificial Sequence
  Description of Artificial Sequence Synthetic peptide
AKYK

SEQ ID NO: 19 - protein, 10 aa, Artificial Sequence
  Description of Artificial Sequence Synthetic peptide
QWVWAVGHLM

SEQ ID NO: 20 - protein, 8 aa, Artificial Sequence
  Description of Artificial Sequence Synthetic peptide
FCFWKTCT

SEQ ID NO: 21 - protein, 4 aa, Artificial Sequence
  Description of Artificial Sequence Synthetic peptide
YKEK

SEQ ID NO: 22 - protein, 8 aa, Artificial Sequence
  Description of Artificial Sequence Synthetic peptide
FCFWKTCT

SEQ ID NO: 23 - protein, 8 aa, Artificial Sequence
  Description of Artificial Sequence Synthetic peptide
QWAVGHLM

SEQ ID NO: 24 - protein, 4 aa, Artificial Sequence
  Description of Artificial Sequence Synthetic peptide
AKYK

SEQ ID NO: 25 - protein, 3 aa, Artificial Sequence
  Description of Artificial Sequence Synthetic peptide
KYK

SEQ ID NO: 26 - protein, 3 aa, Artificial Sequence
  Description of Artificial Sequence Synthetic peptide
KYK

SEQ ID NO: 27 - protein, 4 aa, Artificial Sequence
  Description of Artificial Sequence Synthetic peptide
EKYK

SEQ ID NO: 28 - protein, 4 aa, Artificial Sequence
  Description of Artificial Sequence Synthetic peptide
EKYK

SEQ ID NO: 29 - protein, 3 aa, Artificial Sequence
  Description of Artificial Sequence Synthetic peptide
KYK

SEQ ID NO: 30 - protein, 3 aa, Artificial Sequence
  Description of Artificial Sequence Synthetic peptide
KYK

SEQ ID NO: 31 - protein, 5 aa, Artificial Sequence
  Description of Artificial Sequence Synthetic peptide
GGGGS

SEQ ID NO: 32 - protein, 4 aa, Artificial Sequence
  Description of Artificial Sequence Synthetic peptide
AKYK

(19) United States
(12) Patent Application Publication
(10) Pub. No.: US 2016/0045626 A1
(43) Pub. Date: Feb. 18, 2016

(54) Methods and Compositions for Improved Labeling of Targeting Peptides

(71) Applicant: Immunomedics, Inc., Morris Plains, NJ (US)

(72) Inventors: William J. McBride, Boonton, NJ (US); David M. Goldenberg, Mendham, NJ (US)

(21) Appl. No.: 14/922,619

(22) Filed: Oct. 26, 2015

Related U.S. Application Data
(63) Continuation-in-part of application No. 14/755,712, filed on Jun. 30, 2015.
Application No. 14/755,712 is a division of application No. 14/509,679, filed on Oct. 8, 2014, now Pat. No. 9,115,172.
Application No. 14/509,679 is a division of application No. 14/199,625, filed on Mar. 6, 2014, now Pat. No. 8,889,100.
Application No. 14/199,625 is a division of application No. 13/897,849, filed on May 20, 2013, now Pat. No. 8,709,382.
Application No. 13/897,849 is a continuation-in-part of application No. 13/850,591, filed on Mar. 26, 2013, now Pat. No. 8,617,518.
Application No. 13/850,591 is a division of application No. 13/474,260, filed on May 17, 2012, now Pat. No. 8,444,956.
Application No. 13/474,260 is a division of application No. 12/958,889, filed on Dec. 2, 2010, now Pat. No. 8,202,509.
Application No. 12/958,889 is a continuation-in-part of application No. 12/433,212, filed on Apr. 30, 2009, now Pat. No. 8,153,100.
Application No. 12/433,212 is a continuation-in-part of application No. 12/343,655, filed on Dec. 24, 2008, now Pat. No. 7,993,626.
Application No. 12/343,655 is a continuation-in-part of application No. 12/112,289, filed on Apr. 30, 2008, now Pat. No. 7,563,433.
Application No. 12/112,289 is a continuation-in-part of application No. 11/960,262, filed on Dec. 19, 2007, now Pat. No. 7,597,876.
(60) Provisional application No. 62/078,657, filed on Nov. 12, 2014.
(60) Provisional application No. 60/884,521, filed on Jan. 11, 2007.

(51) Int. Cl. A61K 51/08; A61K 45/06

(52) U.S. Cl. CPC A61K 51/083; A61K 45/06

(57) ABSTRACT

The present application discloses compositions and methods of synthesis and use of labeled targeting peptides, such as octreotide, octreotate, or other somatostatin analogs or derivatives. The targeting peptide may be labeled with a therapeutic or diagnostic isotope, such as ⁶¹Cu, ⁶²Cu, ⁶⁴Cu, ⁶⁷Cu, ¹⁸F, ¹⁹F, ⁶⁶Ga, ⁶⁷Ga, ⁶⁸Ga, ⁷²Ga, ¹¹¹In, ¹⁷⁷Lu, ⁴⁴Sc, ⁴⁷Sc, ⁸⁶Y, ⁸⁸Y, ⁹⁰Y, ⁴⁵Ti or ⁸⁹Zr, preferably ¹⁸F or ¹⁹F. More preferably, the targeting peptide is NOTA-octreotate, NOTA-MPAA-octreotate, pyridine-NOTA-octreotate or triazole-NOTA-octreotate. The labeled targeting peptides may be used for detection, diagnosis, imaging and/or treatment of sst₂⁺ tumors, such as neuroendocrine tumors.

### RELATED APPLICATIONS

This application claims the benefit under 35 U.S.C. 119(e) of provisional U.S. Patent Appl. No. 62/078,657, filed Nov. 12, 2014. This application is a continuation-in-part of U.S. patent application Ser. No. 14/755,712, filed Jun. 30, 2015, which was a divisional of U.S. patent application Ser. No. 14/509,679 (now U.S. Pat. No. 9,115,172), filed Oct. 8, 2014, which was a divisional of Ser. No. 14/199,625 (now U.S. Pat. No. 8,889,100), filed Mar. 6, 2014, which was a divisional of Ser. No. 13/897,849 (now U.S. Pat. No. 8,709,382), filed May 20, 2013, which was a continuation-in-part of Ser. No. 13/850,591 (now U.S. Pat. No. 8,617,518), filed Mar. 26, 2013, which was a divisional of Ser. No. 13/474,260 (now U.S. Pat. No. 8,444,956), filed May 17, 2012, which was a divisional of Ser. No. 12/958,889 (now U.S. Pat. No. 8,202,509), filed Dec. 2, 2010, which was a continuation-in-part of Ser. No. 12/433,212 (now U.S. Pat. No. 8,153,100), filed Apr. 30, 2009, which was a continuation-in-part of Ser. No. 12/343,655 (now U.S. Pat. No. 7,993,626), filed Dec. 24, 2008, which was a continuation-in-part of Ser. No. 12/112,289 (now U.S. Pat. No. 7,563,433), filed Apr. 30, 2008, which was a continuation-in-part of Ser. No. 11/960,262 (now U.S. Pat. No. 7,597,876), filed Dec. 19, 2007, which claimed the benefit under 35 U.S.C. 119(e) of provisional U.S. Patent Appl. 60/884,521, filed Jan. 11, 2007. The text of each priority application is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Oct. 9, 2015 is named IMM351US1_SL and is 14,929 bytes in size.

### FIELD

The present invention concerns novel compounds comprising octreotide, octreotate, or another somatostatin analog, of use for targeted delivery to cells or tissues expressing somatostatin receptors. The compounds may be used to deliver diagnostic agents for detection, diagnosis or imaging, or therapeutic agents for treatment of diseased cells or tissues that express somatostatin receptor, such as neuroendocrine tumors. The compounds further comprise one or more chelator moieties, which may be used to attach diagnostic or therapeutic radionuclides, paramagnetic ions or other diagnostic or therapeutic agents. In more preferred embodiments, the compounds may be labeled with metal-¹⁸F or metal-¹⁹F complexes that are of use, for example, in PET, MRI or SPECT in vivo imaging. Preferably, the ¹⁸F or ¹⁹F is attached as a complex with aluminum or another Group IIIA metal. The chelating moiety may be attached to the targeting peptide either before or after binding to the metal-¹⁸F or metal-¹⁹F complex. Although labeling may occur at an elevated temperature, such as 70° C., 80° C., 90° C., 95° C., 100° C., 105° C., 110° C., or any temperature in between, preferably labeling of heat sensitive molecules may occur at a lower temperature, such as room temperature. Although fluorine isotopes are preferred, in alternative embodiments other therapeutic or diagnostic metals and/or isotopes may be bound to the chelating moieties discussed below, including but not limited to isotopes of aluminum, gallium, indium, copper and yttrium. Using the techniques described herein, labeled molecules of high specific activity may be prepared in 30 minutes or less and with minimal or no need for purification of labeled molecules. Labeling may occur in a saline medium suitable for direct use in vivo. Alternatively, an organic solvent may be added to improve the labeling efficiency. The labeled targeting peptides are stable under physiological conditions, although for certain purposes, such as kit formulations, a stabilizing agent such as ascorbic acid, trehalose, sorbitol or mannitol may be added.

### BACKGROUND

Octreotate is an octapeptide that mimics somatostatin and binds with high affinity to somatostatin receptors. Octreotide is a structurally similar peptide wherein the C-terminal threonine moiety has been reduced to the corresponding amino alcohol. Both have been extensively used for detection, imaging or treatment of diseased tissues that express high levels of somatostatin receptor, particularly neuroendocrine tumors or other somatostatin-receptor positive endocrine tumors (e.g., Bodei et al., 2014, Thorac Surg Clin 24:333-49; van Essen et al., 2009, Nat Rev Endocrinol 5:382-93; De Jong et al., 2002, Semn Nucl Med 32:133-40). Therapeutic or diagnostic agents that have been attached to octreotide or octreotate for delivery to targeted tissues have included ⁹⁰Y, ¹⁷⁷Lu, ¹¹¹In, ⁶⁸Ga, ¹²³I, ¹¹C, ²¹³Bi, and ²¹¹At, (see, Bodei et al., 2014, Thorac Surg Clin 24:333-49; van Essen et al., 2009, Nat Rev Endocrinol 5:382-93; Kwekkeboom et al., 2010, Endocr Relat Cancer 17:R53-73; Chin et al., 2013, Amino Acids 45:1097-108; Dadachova, 2010, Semin Nucl Med 40:204-8).

Although symptomatic improvement has been reported with such conjugated somatostatin analogs, large variation in antitumor effects has been reported in different studies with, for example, an objective response achieved in 9% to 33% of patients treated with ⁹⁰Y-octreotide (van Essen et al., 2009, Nat Rev Endocrinol 5:382-93). A need exists for more effective somatostatin analogs that have better labeling characteristics, improved stability and/or increased therapeutic or diagnostic efficiency with better imaging.

### SUMMARY

In various embodiments, the present invention concerns compositions and methods relating to labeled octreotide, octreotate or other somatostatin analogs, of use for targeted delivery to cells or tissues expressing somatostatin receptors (sst). The major subtype of sst is sst₂, and therapeutic or diagnostic uses of octreotide or octreotate have been primarily directed to sst₂⁺ cancers (e.g., Lustig et al., 2003, J Clin Endocrinol 88:2586-92; Uhl et al., 1999, Digestion 60(Suppl 2):23-31). Within the scope of the present invention for therapy and/or diagnosis, labeled octreotide or octreotate may be applied to tumors including, but not limited to, sst₂ neuroendocrine tumors (NET), gastroenteropancreatic NET, meningiomas, well-differentiated brain tumors, malignant lymphomas, renal cell carcinoma, breast carcinoma and lung carcinoma. Any other cancer that is sst₂⁺ may also be treated or diagnosed.

Exemplary radionuclides or stable isotopes that may be attached to the subject peptides for therapy and/or diagnosis include, but are not limited to, ⁶¹Cu, ⁶²Cu, ⁶⁴Cu, ⁶⁷Cu, ¹⁸F, ¹⁹F, ⁶⁶Ga, ⁶⁷Ga, ⁶⁸Ga, ⁷²Ga, ¹¹¹In, ¹⁷⁷Lu, ⁴⁴Sc, ⁴⁷Sc, ⁸⁶Y, ⁸⁸Y, ⁹⁰Y, ⁴⁵Ti and ⁸⁹Zr.

In preferred embodiments, the radionuclides or other diagnostic or therapeutic agents may be attached to octreotide, octreotate or other somatostatin analogs using a chelating moiety, such as NOTA, NETA, DOTA, DTPA or derivatives thereof. Exemplary chelating moieties of particular use are shown below and in the Examples section. The person of ordinary skill will understand that the chelating moieties of use are not limited to the specific embodiments disclosed herein, but rather may include other chelating moieties known in the art to bind therapeutic and/or diagnostic agents.

In particularly preferred embodiments, targeting peptides of use may include, but are not limited to the NOTA-octreotate derivatives shown below. The underlined portion of the peptide may be cyclized by disulfide bond formation between the two cysteine residues.

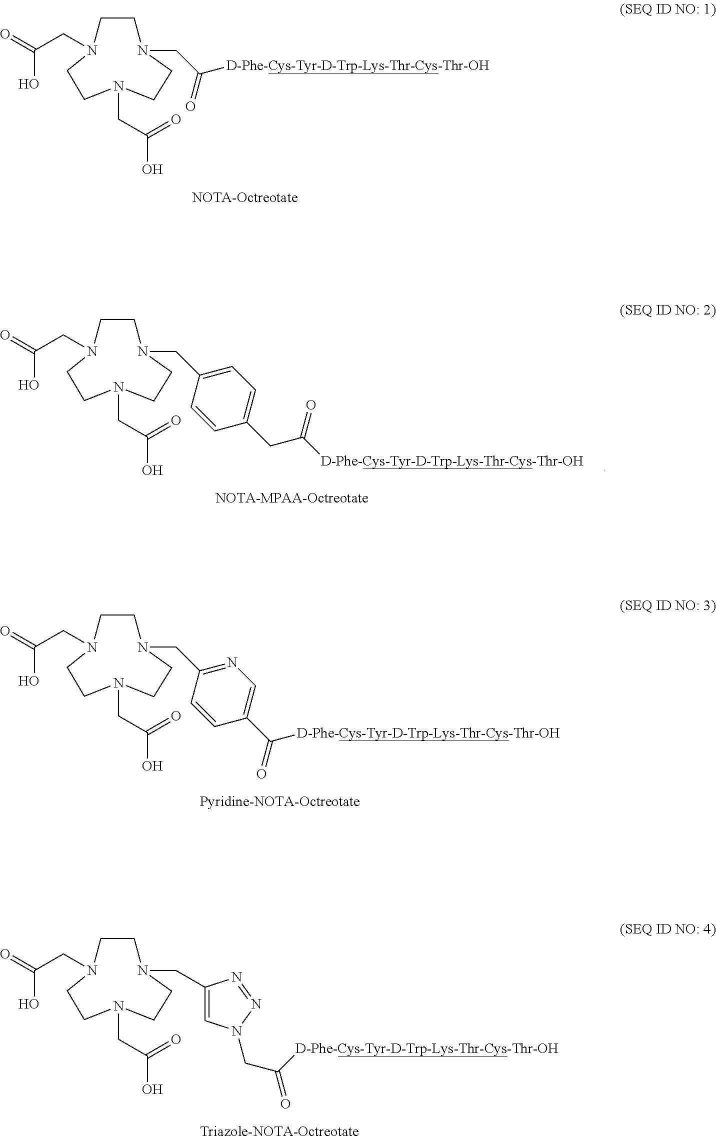

Although the Examples below show use of radionuclide-labeled chelator-peptide complexes, the invention is not limited and other diagnostic or therapeutic agents known to bind to chelating moieties may be utilized within the scope of the claimed methods and compositions.

In preferred embodiments, the chelating moieties are used to attach metal-¹⁸F or metal-¹⁹F complexes to octreotide, octreotate or other somatostatin analogs. In an exemplary approach, the ¹⁸F is bound to a metal and the ¹⁸F-metal complex is attached to a chelator on the peptide. As described below, the metals of group IIIA (aluminum, gallium, indium, and thallium) are suitable for ¹⁸F or ¹⁹F binding, although aluminum is preferred. Lutetium may also be of use. The chelating moiety be selected from NOTA, NETA, DOTA, DTPA and other chelating groups discussed in more detail below. Alternatively, one can attach the metal to a molecule first and then add the ¹⁸F to bind to the metal. In still other embodiments, one may attach an ¹⁸F-metal to a chelating moiety first and then attach the labeled chelating moiety to the peptide. In this way, the ¹⁸F-metal may be attached to a chelating moiety at a higher temperature, such as between 90° to 110° C., more preferably between 95° to 105° C., and the ¹⁸F-labeled chelating moiety may be attached to the peptide at a lower temperature, such as at room temperature. In preferred embodiments, the labeling method uses a biofunctional chelator that forms a physiologically stable complex with metal-¹⁸F, which contains reactive groups that can bind to peptides at, e.g., room temperature. More preferably, labeling can be accomplished in 10 to 15 minutes in aqueous medium, with a total synthesis time of about 30 minutes.

Certain alternative embodiments involve the use of “click” chemistry for attachment of ¹⁸F-labeled moieties to targeting molecules. Preferably, the click chemistry involves the reaction of a targeting peptide comprising a functional group such as an alkyne, nitrone or an azide group, with a ¹⁸F-labeled moiety comprising the corresponding reactive moiety such as an azide, alkyne or nitrone. Where the targeting molecule comprises an alkyne, the chelating moiety or carrier will comprise an azide, a nitrone or similar reactive moiety.

In other alternative embodiments, a prosthetic group, such as a NOTA-maleimide moiety, may be labeled with ¹⁸F-metal and then conjugated to a targeting molecule, for example by a maleimide-sulfhydryl reaction. Exemplary NOTA-maleimide moieties include, but are not limited to, NOTA-MPAEM, NOTA-PM, NOTA-PAEM, NOTA-BAEM, NOTA-BM, NOTA-MPM, and NOTA-MBEM.

The claimed compounds may be used in combination with other standard therapeutic modalities, such as surgery, chemotherapy, radiation therapy, immunotherapy and the like. Labeled somatostatin analogs may also be utilized in adjuvant or neoadjuvant settings. The therapeutic efficacy of the labeled somatostatin analogs may be enhanced by combination therapy with other therapeutic agents, administered either before, concurrently with or after the labeled somatostatin analogs. Agents of use in combination therapy may include, but are not limited to, canertinib, dasatinib, erlotinib, gefitinib, imatinib, lapatinib, leflunomide, nilotinib, pazopanib, semaxinib, sorafenib, sunitinib, vatalanib, temsirolimus, rapamycin, ridaforolimus everolimus, ibrutinib, 5-fluorouracil, capecitabine, temozolomide, lambrolizumab, pidilizumab, ipilimumab and tremelimumab.

### BRIEF DESCRIPTION OF THE DRAWINGS

The following Figures are included to illustrate particular embodiments of the invention and are not meant to be limiting as to the scope of the claimed subject matter.

FIG. 1. Biodistribution of ¹⁸F-labeled agents in tumor-bearing nude mice by microPET imaging. Coronal slices of 3 nude mice bearing a small, subcutaneous LS174T tumor on each left flank after being injected with either (A) ¹⁸F-FDG, (B) Al¹⁸F(IMP449) pretargeted with the anti-CEA×anti-HSG bsMAb, (C) Al¹⁸F(IMP449) alone (not pretargeted with the bsMAb). Biodistribution data expressed as percent-injected dose per gram (% ID/g) are given for the tissues removed from the animals at the conclusion of the imaging session. Abbreviations: B, bone marrow; H, heart; K, kidney; T, tumor.

FIG. 2. Dynamic imaging study of pretargeted Al¹⁸F(IMP449) given to a nude mouse bearing a 35-mg LS174T human colorectal cancer xenograft in the upper flank. The top 3 panels show coronal, sagittal, and transverse sections, respectively, taken of a region of the body centering on the tumor's peripheral location at 6 different 5-min intervals over the 120-min imaging session. The first image on the left in each sectional view shows the positioning of the tumor at the intersection of the crosshairs, which is highlighted by arrows. The animal was partially tilted to its right side during the imaging session. The bottom 2 panels show additional coronal and sagittal sections that focus on a more anterior plane in the coronal section to highlight distribution in the liver and intestines, while the sagittal view crosses more centrally in the body. Abbreviations: Cor, coronal; FA, forearms; H, heart; K, kidney; Lv, liver; Sag, sagittal; Tr, transverse; UB, urinary bladder.

FIG. 3. In vivo tissue distribution with Al¹⁸F(IMP466) bombesin analogue.

FIG. 4. Comparison of biodistribution of Al¹⁸F(IMP466) and ⁶⁸Ga(IMP466) at 2 hours post-injection in AR42J tumor-bearing mice (n=5). As a control, mice in separate groups (n=5) received an excess of unlabeled octreotide to demonstrate receptor specificity.

FIG. 5. Coronal slices of PET/CT scan of Al¹⁸F(IMP466) and ⁶⁸Ga(IMP466) at 2 hours post-injection in mice with an s.c. AR42J tumor in the neck. Accumulation in tumor and kidneys is clearly visualized.

FIG. 6. Biodistribution of 6.0 nmol ¹²⁵I-TF2 (0.37 MBq) and 0.25 nmol ⁶⁸Ga(IMP288) (5 MBq), 1 hour after i.v. injection of ⁶⁸Ga(IMP288) in BALB/c nude mice with a subcutaneous LS174T and SK-RC52 tumor. Values are given as means±standard deviation (n=5).

FIG. 7. Biodistribution of 5 MBq FDG and of 5 MBq ⁶⁸Ga(IMP288) (0.25 nmol) 1 hour after i.v. injection following pretargeting with 6.0 nmol TF2. Values are given as means±standard deviation (n=5).

FIG. 8. PET/CT images of a BALB/c nude mouse with a subcutaneous LS 174T tumor (0.1 g) on the right hind leg (light arrow) and a inflammation in the left thigh muscle (dark arrow), that received 5 MBq ¹⁸F-FDG, and one day later 6.0 nmol TF2 and 5 MBq ⁶⁸Ga(IMP288) (0.25 nmol) with a 16 hour interval. The animal was imaged one hour after the ¹⁸F-FDG and ⁶⁸Ga(IMP288) injection. The panel shows the 3D volume rendering (A), transverse sections of the tumor region (B) of the FDG-PET scan, and the 3D volume rendering (C), transverse sections of the tumor region (D) of the pretargeted immunoPET scan.

FIG. 9. Biodistribution of 0.25 nmol Al¹⁸F(IMP449) (5 MBq) 1 hour after i.v. injection, following 6.0 nmol TF2 administered 16 hours earlier. Biodistribution of Al¹⁸F(IMP449) without pretargeting, or biodistribution of [Al¹⁸F]. Values are given as means±standard deviation.

FIG. 10. Static PET/CT imaging study of a BALB/c nude mouse with a subcutaneous LS174T tumor (0.1 g) on the right side (arrow), that received 6.0 nmol TF2 and 0.25 nmol Al¹⁸F(IMP449) (5 MBq) intravenously with a 16 hour interval. The animal was imaged one hour after injection of Al¹⁸F(IMP449). The panel shows the 3D volume rendering (A) posterior view, and cross sections at the tumor region, (B) coronal, (C) sagittal.

FIG. 11. Structure of IMP479 (SEQ ID NO:24).

FIG. 12. Structure of IMP485 (SEQ ID NO:25).

FIG. 13A. Structure of IMP487 (SEQ ID NO:26).

FIG. 13B. Structure of IMP490 (SEQ ID NO:22).

FIG. 13C. Structure of IMP493 (SEQ ID NO:23).

FIG. 13D. Structure of IMP495 (SEQ ID NO: 27).

FIG. 13E. Structure of IMP496 (SEQ ID NO: 28).

FIG. 13F. Structure of IMP500.

FIG. 14. Synthesis of bis-t-butyl-NOTA-MPAA.

FIG. 15. Synthesis of maleimide conjugate of NOTA.

FIG. 16. Chemical structure of exemplary NOTA-based bifunctional chelators.

FIG. 17. Chemical structures of NOTA-BM derived bifunctional chelators.

FIG. 18. Further exemplary structures of NOTA-based bifunctional chelators: (A) NOTA-HA, (B) NOTA-MPN, (C) NOTA-EPN, (D) NOTA-MBA, (E) NOTA-EPA, (F) NOTA-MPAA, (G) NOTA-BAEM, (H) NOTA-MPAEM, (I) NOTA-BM, (J) NOTA-MBEM, (K) NOTA moiety with maleimide reactive group, (L) alternative NOTA moiety with maleimide reactive group, (M) NOTA-BA, (N) NOTA-EA, (O) NOTA-MPH, (P) NOTA-butyne, (Q) NOTA-MPAPEG₃N₃, (R) NOTA moiety with carboxyl reactive group, (S) NOTA moiety with nitrophenyl reactive group, (T) NOTA moiety with carboxyl and nitrophenyl reactive groups, (U) another NOTA moiety with carboxyl reactive group, (V) another NOTA moiety with carboxyl reactive group, (W) another NOTA moiety with carboxyl reactive group, (X) another NOTA moiety with carboxyl reactive group, (Y) another NOTA moiety with carboxyl reactive group, (Z) another NOTA moiety with carboxyl reactive group, (AA) another NOTA moiety with carboxyl reactive group, (BB) another NOTA moiety with carboxyl reactive group, (CC) another NOTA moiety with carboxyl reactive group.

FIGS. 19(A) and 19(B). Radiochromatograms of the ¹⁸F-labeled functionalized TACN ligands.

FIGS. 20(A) and 20(B). Radiochromatograms of ¹⁸F-hMN14-Fab′, its stability in human serum and immunoreactivity with CEA.

FIG. 21. Schematic diagram of automated synthesis module for ¹⁸F-labeling via [Al¹⁸F]-chelation.

FIG. 22. NOTA-propyl amine derived bifunctional chelating moieties.

FIG. 23A. Structure of IMP 508 (SEQ ID NO: 29).

FIG. 23B. Structure of IMP517 (SEQ ID NO: 30).

FIG. 23C. Structure of NOTA-2-nitroimidazole.

FIG. 23D. Structure of NOTA-DUPA-Peptide.

FIG. 24. Labeling efficiency as a function of temperature.

### DETAILED DESCRIPTION

The following definitions are provided to facilitate understanding of the disclosure herein. Terms that are not explicitly defined are used according to their plain and ordinary meaning

As used herein, the term “somatostatin analog(s)” refers to octreotide, octreatate, or other derivatives or analogs of somatostatin.

As used herein, “a” or “an” may mean one or more than one of an item.

As used herein, the terms “and” and “or” may be used to mean either the conjunctive or disjunctive. That is, both terms should be understood as equivalent to “and/or” unless otherwise stated.

As used herein, “about” means within plus or minus ten percent of a number. For example, “about 100” would refer to any number between 90 and 110.

As used herein, a “peptide” refers to any sequence of naturally occurring or non-naturally occurring amino acids of between 2 and 100 amino acid residues in length, more preferably between 2 and 10, more preferably between 4 and 8 amino acids in length. An “amino acid” may be an L-amino acid, a D-amino acid, an amino acid analogue, an amino acid derivative or an amino acid mimetic.

As used herein, a “radiolysis protection agent” refers to any molecule, compound or composition that may be added to a radionuclide-labeled complex or molecule to decrease the rate of breakdown of the radiolabeled complex or molecule by radiolysis. Any known radiolysis protection agent, including but not limited to ascorbic acid, may be used.

### Peptides

The targeting peptides used are conveniently synthesized on an automated peptide synthesizer using a solid-phase support and standard techniques of repetitive orthogonal deprotection and coupling. Free amino groups in the peptide, that are to be used later for conjugation of chelating moieties or other agents, are advantageously blocked with standard protecting groups such as a Boc group, while N-terminal residues may be acetylated to increase serum stability. Such protecting groups are well known to the skilled artisan. See Greene and Wuts Protective Groups in Organic Synthesis, 1999 (John Wiley and Sons, N.Y.). Peptides are advantageously cleaved from the resins to generate the corresponding C-terminal amides, in order to inhibit in vivo carboxypeptidase activity. Exemplary structures of use and methods of peptide synthesis are disclosed in the Examples below. Chelating moieties may be conjugated to peptides using bifunctional chelating moieties as discussed below.

### Amino Acid Substitutions

Certain embodiments may involve production and use of targeting peptides with one or more substituted amino acid residues. The skilled artisan will be aware that amino acid substitutions typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within ±2 is preferred, within ±1 are more preferred, and within ±0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Some embodiments may involve substitution of one or more D-amino acids for the corresponding L-amino acids. Peptides comprising D-amino acid residues are more resistant to peptidase activity than L-amino acid comprising peptides. Such substitutions may be readily performed using standard amino acid synthesizers, as discussed in the Examples below.

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded protein sequence are well known and a matter of routine experimentation for the skilled artisan, for example by the technique of site-directed mutagenesis or by synthesis and assembly of oligonucleotides encoding an amino acid substitution and splicing into an expression vector construct.

### Chelating Moieties

In some embodiments, Al¹⁸F or another radiolabel may bind to a hydrophilic chelating moiety, which can bind metal ions and also help to ensure rapid in vivo clearance. Chelators may be selected for their particular metal-binding properties, and substitution by known chemical cross-linking techniques or by use of chelators with side-chain reactive groups (such as bifunctional chelating moieties) may be performed with only routine experimentation.

Particularly useful metal-chelate combinations include 2-benzyl-DTPA and its monomethyl and cyclohexyl analogs, used with diagnostic isotopes in the general energy range of 60 to 4,000 keV, such as ¹²⁵I, ¹³¹I, ¹²³I, ¹²⁴I, ⁶²Cu, ⁶⁴Cu, ¹⁸F, ¹¹¹In, ⁶⁷Ga, ⁶⁸Ga, ^{99m}Tc, ^{94m}Tc, ¹¹C, ¹³N, ¹⁵O, ⁷⁶Br, for radioimaging. The same chelates, when complexed with non-radioactive metals, such as manganese, iron and gadolinium are useful for MRI. Macrocyclic chelates such as NOTA (1,4,7-triazacyclononane-1,4,7-triacetic acid), DOTA, TETA (p-bromoacetamido-benzyl-tetraethylaminetetraacetic acid) and NETA are of use with a variety of diagnostic or therapeutic metals and radiometals, most particularly with radionuclides of gallium, yttrium and copper. Such metal-chelate complexes can be made very stable by tailoring the ring size to the metal of interest. Other ring-type chelates such as macrocyclic polyethers, which are of interest for stably binding nuclides, such as ²²³Ra for RAIT are encompassed. The person of ordinary skill will understand that, by varying the groups attached to a macrocyclic ring structure such as NOTA, the binding characteristics and affinity for different metals and/or radionuclides may change and such derivatives or analogs of, e.g. NOTA, may therefore be designed to bind any of the metals, radionuclides and/or paramagnetic species discussed herein.

DTPA and DOTA-type chelators, where the ligand includes hard base chelating functions such as carboxylate or amine groups, are most effective for chelating hard acid cations, especially Group IIa and Group IIIa metal cations. Such metal-chelate complexes can be made very stable by tailoring the ring size to the metal of interest. Other ring-type chelators such as macrocyclic polyethers are of interest for stably binding nuclides. Porphyrin chelators may be used with numerous metal complexes. More than one type of chelator may be conjugated to a peptide to bind multiple metal ions. Chelators such as those disclosed in U.S. Pat. No. 5,753,206, especially thiosemicarbazonylglyoxylcysteine (Tscg-Cys) and thiosemicarbazinyl-acetylcysteine (Tsca-Cys) chelators are advantageously used to bind soft acid cations of Tc, Re, Bi and other transition metals, lanthanides and actinides that are tightly bound to soft base ligands. One example of such a peptide is Ac-Lys(DTPA)-Tyr-Lys(DTPA)-Lys(Tscg-Cys)-NH₂ (core peptide disclosed as SEQ ID NO:5). Other hard acid chelators such as DOTA, TETA and the like can be substituted for the DTPA and/or Tscg-Cys groups.

Another useful chelator may comprise a NOTA-type moiety, for example as disclosed in Chong et al. (J. Med. Chem., 2008, 51:118-25). Chong et al. disclose the production and use of a bifunctional C-NETA ligand, based upon the NOTA structure, that when complexed with ¹⁷⁷Lu or ^{205/206}Bi showed stability in serum for up to 14 days. The chelators are not limiting and these and other examples of chelators that are known in the art and/or described in the following Examples may be used in the practice of the invention.

### Click Chemistry

In various embodiments, targeting peptide conjugates may be prepared using click chemistry technology. The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems. However, the copper catalyst is toxic to living cells, precluding biological applications.

A copper-free click reaction has been proposed for covalent modification of biomolecules in living systems. (See, e.g., Agard et al., 2004, J Am Chem Soc 126:15046-47.) The copper-free reaction uses ring strain in place of the copper catalyst to promote a [3+2] azide-alkyne cycloaddition reaction (Id.) For example, cyclooctyne is a 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions, without the toxic copper catalyst (Id.)

Another type of copper-free click reaction was reported by Ning et al. (2010, Angew Chem Int Ed 49:3065-68), involving strain-promoted alkyne-nitrone cycloaddition. To address the slow rate of the original cyclooctyne reaction, electron-withdrawing groups are attached adjacent to the triple bond (Id.) Examples of such substituted cyclooctynes include difluorinated cyclooctynes, 4-dibenzocyclooctynol and azacyclooctyne (Id.) An alternative copper-free reaction involved strain-promoted alkyne-nitrone cycloaddition to give N-alkylated isoxazolines (Id.) The reaction was reported to have exceptionally fast reaction kinetics and was used in a one-pot three-step protocol for site-specific modification of peptides and proteins (Id.) Nitrones were prepared by the condensation of appropriate aldehydes with N-methylhydroxylamine and the cycloaddition reaction took place in a mixture of acetonitrile and water (Id.)

The Diels-Alder reaction has also been used for in vivo labeling of molecules. Rossin et al. (2010, Angew Chem Int Ed 49:3375-78) reported a 52% yield in vivo between a tumor-localized anti-TAG72 (CC49) antibody carrying a trans-cyclooctene (TCO) reactive moiety and an ¹¹¹ In-labeled tetrazine DOTA derivative. The TCO-labeled CC49 antibody was administered to mice bearing colon cancer xenografts, followed 1 day later by injection of ¹¹¹ In-labeled tetrazine probe (Id.) The reaction of radiolabeled probe with tumor localized antibody resulted in pronounced radioactivity localized in the tumor, as demonstrated by SPECT imaging of live mice three hours after injection of radiolabeled probe, with a tumor-to-muscle ratio of 13:1 (Id.) The results confirmed the in vivo chemical reaction of the TCO and tetrazine-labeled molecules.

Modifications of click chemistry reactions are suitable for use in vitro or in vivo. Reactive targeting molecule may be formed either by either chemical conjugation or by biological incorporation. The targeting peptide may be activated with an azido moiety, a substituted cyclooctyne or alkyne group, or a nitrone moiety. Where the targeting peptide comprises an azido or nitrone group, the corresponding chelator will comprise a substituted cyclooctyne or alkyne group, and vice versa. Such activated molecules may be made by metabolic incorporation in living cells, as discussed above. Alternatively, methods of chemical conjugation of such moieties to biomolecules are well known in the art, and any such known method may be utilized. The disclosed techniques may be used in combination with the diagnostic radionuclide (e.g., ¹⁸F) labeling methods described below for PET, SPECT or MRI imaging, or alternatively may be utilized for delivery of any therapeutic and/or diagnostic agent that may be attached to a suitable activated targeting peptide.

Therapeutic Agents

In varioius embodiments, the labeled targeting peptides may be administered in combination with one or more additional therapeutic or diagnostic agents. Such additional agents may be administered before, concurrently with, or after the labeled peptide. Therapeutic agents of use may include cytotoxic agents, anti-angiogenic agents, pro-apoptotic agents, antibiotics, hormones, hormone antagonists, chemokines, drugs, prodrugs, toxins, enzymes, antibodies, antibody fragments, immunoconjugates, immunomodulators, oligonucleotides, siRNA, RNAi or other known agents.

Drugs of use may possess a pharmaceutical property selected from the group consisting of antimitotic, antikinase, alkylating, antimetabolite, antibiotic, alkaloid, anti-angiogenic, pro-apoptotic agents and combinations thereof. Exemplary drugs of use include, but are not limited to, 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, Cox-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, doxorubicin, 2-pyrrolinodoxorubicine (2P-DOX), pro-2P-DOX, cyano-morpholino doxorubicin, doxorubicin glucuronide, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide (an aqueous form of DTIC), transplatinum, thalidomide, thioguanine, thiotepa, teniposide, topotecan, uracil mustard, vatalanib, vinorelbine, vinblastine, vincristine, vinca alkaloids and ZD1839.

Toxins of use may include ricin, abrin, alpha toxin, saporin, ribonuclease (RNase), e.g., onconase, DNase I, Staphylococcal enterotoxin-A, pokeweed antiviral protein, gelonin, diphtheria toxin, Pseudomonas exotoxin, and Pseudomonas endotoxin.

Chemokines of use may include RANTES, MCAF, MIP 1-alpha, MIP 1-Beta and IP-10.

In certain embodiments, anti-angiogenic agents, such as angiostatin, baculostatin, canstatin, maspin, anti-VEGF antibodies, anti-PlGF peptides and antibodies, anti-vascular growth factor antibodies, anti-Flk-1 antibodies, anti-Flt-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, IP-10, Gro-β, thrombospondin, 2-methoxyoestradiol, proliferin-related protein, carboxiamidotriazole, CM101, Marimastat, pentosan polysulphate, angiopoietin-2, interferon-alpha, herbimycin A, PNU145156E, 16K prolactin fragment, Linomide (roquinimex), thalidomide, pentoxifylline, genistein, TNP-470, endostatin, paclitaxel, accutin, angiostatin, cidofovir, vincristine, bleomycin, AGM-1470, platelet factor 4 or minocycline may be of use.

Immunomodulators of use may be selected from a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β or -γ, and stem cell growth factor, such as that designated “S1 factor”. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin, tumor necrosis factor and LT.

Radionuclides of use include, but are not limited to—¹¹¹In, ¹⁷⁷Lu, ²¹²Bi, ²¹³Bi, ²¹¹At, ⁶²Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ²²⁷Th, and ²¹¹Pb. The therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, 1-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227 and Fm-255. Decay energies of useful alpha-particle-emitting radionuclides are preferably 2,000-10,000 keV, more preferably 3,000-8,000 keV, and most preferably 4,000-7,000 keV. Additional potential radioisotopes of use include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like. Some useful diagnostic nuclides may include ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ⁹⁴Tc, ^{94m}Tc, ^{99m}Tc, or ¹¹¹In.

Therapeutic agents may include a photoactive agent or dye. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See Joni et al. (eds.), PHOTODYNAMIC THERAPY OF TUMORS AND OTHER DISEASES (Libreria Progetto 1985); van den Bergh, Chem. Britain (1986), 22:430. Moreover, targeting molecules have been coupled with photoactivated dyes for achieving phototherapy. See Mew et al., J. Immunol. (1983),130:1473; idem., Cancer Res. (1985), 45:4380; Oseroff et al., Proc. Natl. Acad. Sci. USA (1986), 83:8744; idem., Photochem. Photobiol. (1987), 46:83; Hasan et al., Prog. Clin. Biol. Res. (1989), 288:471; Tatsuta et al., Lasers Surg. Med. (1989), 9:422; Pelegrin et al., Cancer (1991), 67:2529.

Other useful therapeutic agents may comprise oligonucleotides, especially antisense oligonucleotides that preferably are directed against oncogenes and oncogene products, such as bcl-2 or p53. A preferred form of therapeutic oligonucleotide is siRNA.

Diagnostic Agents

Diagnostic agents are preferably selected from the group consisting of a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters. Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerytherin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

### Methods of Administration

The labeled targeting peptides may be formulated to obtain compositions that include one or more pharmaceutically suitable excipients, one or more additional ingredients, or some combination of these. These can be accomplished by known methods to prepare pharmaceutically useful dosages, whereby the active ingredients (i.e., the labeled peptides) are combined in a mixture with one or more pharmaceutically suitable excipients. Sterile phosphate-buffered saline is one example of a pharmaceutically suitable excipient. Other suitable excipients are well known to those in the art. See, e.g., Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The preferred route for administration of the compositions described herein is parenteral injection. Injection may be intravenous, intraarterial, intralymphatic, intrathecal, subcutaneous or intracavitary (i.e., parenterally). In parenteral administration, the compositions will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hank's solution. Nonaqueous excipients such as fixed oils and ethyl oleate may also be used. A preferred excipient is 5% dextrose in saline. The excipient may contain minor amounts of additives such as substances that enhance isotonicity and chemical stability, including buffers and preservatives. Other methods of administration, including oral administration, are also contemplated.

Formulated compositions comprising labeled targeting peptides can be used for intravenous administration via, for example, bolus injection or continuous infusion. Compositions for injection can be presented in unit dosage form, e.g., in ampoules or in multi-dose containers, with an added preservative. Compositions can also take such forms as suspensions, solutions or emulsions in oily or aqueous vehicles, and can contain formulatory agents such as suspending, stabilizing and/or dispersing agents. Alternatively, the compositions can be in powder form for constitution with a suitable vehicle, e.g., sterile pyrogen-free water, before use.

The compositions may be administered in solution. The pH of the solution should be in the range of pH 5 to 9.5, preferably pH 6.5 to 7.5. The formulation thereof should be in a solution having a suitable pharmaceutically acceptable buffer such as phosphate, TRIS (hydroxymethyl) aminomethane-HCl or citrate and the like. In certain preferred embodiments, the buffer is potassium biphthalate (KHP), which may act as a transfer ligand to facilitate ¹⁸F-labeling. Buffer concentrations should be in the range of 1 to 100 mM. The formulated solution may also contain a salt, such as sodium chloride or potassium chloride in a concentration of 50 to 150 mM. An effective amount of a stabilizing agent such as glycerol, albumin, a globulin, a detergent, a gelatin, a protamine or a salt of protamine may also be included. The compositions may be administered to a mammal subcutaneously, intravenously, intramuscularly or by other parenteral routes. Moreover, the administration may be by continuous infusion or by single or multiple boluses.

In general, the dosage of ¹⁸F or other radiolabel to administer to a human subject will vary depending upon such factors as the patient's age, weight, height, sex, general medical condition and previous medical history. Preferably, a saturating dose of the labeled molecule is administered to a patient. For administration of radiolabeled molecules, the dosage may be measured by millicuries. A typical range for imaging studies would be five to 10 mCi.

Administration of Peptides

Various embodiments of the claimed methods and/or compositions may concern one or more ¹⁸F- or other radiolabeled peptides to be administered to a subject. Administration may occur by any route known in the art, including but not limited to oral, nasal, buccal, inhalational, rectal, vaginal, topical, orthotopic, intradermal, subcutaneous, intramuscular, intraperitoneal, intraarterial, intrathecal or intravenous injection.

In certain embodiments, the standard peptide bond linkage may be replaced by one or more alternative linking groups, such as CH₂—NH, CH₂—S, CH₂—CH₂, CH═CH, CO—CH₂, CHOH—CH₂ and the like. Methods for preparing peptide mimetics are well known (for example, Hruby, 1982, Life Sci 31:189-99; Holladay et al., 1983, Tetrahedron Lett. 24:4401-04; Jennings-White et al., 1982, Tetrahedron Lett. 23:2533; Almquiest et al., 1980, J. Med. Chem. 23:1392-98; Hudson et al., 1979, Int. J. Pept. Res. 14:177-185; Spatola et al., 1986, Life Sci 38:1243-49; U.S. Pat. Nos. 5,169,862; 5,539,085; 5,576,423, 5,051,448, 5,559,103.) Peptide mimetics may exhibit enhanced stability and/or absorption in vivo compared to their peptide analogs.

Peptide stabilization may also occur by substitution of D-amino acids for naturally occurring L-amino acids, particularly at locations where endopeptidases are known to act. Endopeptidase binding and cleavage sequences are known in the art and methods for making and using peptides incorporating D-amino acids have been described (e.g., U.S. Patent Application Publication No. 20050025709, McBride et al., filed Jun. 14, 2004, the Examples section of which is incorporated herein by reference).

### Imaging Using Labeled Molecules

Methods of imaging using labeled molecules are well known in the art, and any such known methods may be used with the labeled targeting peptides disclosed herein. See, e.g., U.S. Pat. Nos. 6,241,964; 6,358,489; 6,953,567 and published U.S. Patent Application Publ. Nos. 20050003403; 20040018557; 20060140936, the Examples section of each incorporated herein by reference. See also, Page et al., Nuclear Medicine And Biology, 21:911-919, 1994; Choi et al., Cancer Research 55:5323-5329, 1995; Zalutsky et al., J. Nuclear Med., 33:575-582, 1992; Woessner et. al. Magn. Reson. Med. 2005, 53: 790-99.

Methods of diagnostic imaging with labeled peptides are well-known. For example, in the technique of immunoscintigraphy, peptide ligands are labeled with a gamma-emitting radioisotope and introduced into a patient. A gamma camera is used to detect the location and distribution of gamma-emitting radioisotopes. See, for example, Srivastava (ed.), RADIOLABELED MONOCLONAL ANTIBODIES FOR IMAGING AND THERAPY (Plenum Press 1988), Chase, “Medical Applications of Radioisotopes,” in REMINGTON'S PHARMACEUTICAL SCIENCES, 18th Edition, Gennaro et al. (eds.), pp. 624-652 (Mack Publishing Co., 1990), and Brown, “Clinical Use of Monoclonal Antibodies,” in BIOTECHNOLOGY AND PHARMACY 227-49, Pezzuto et al. (eds.) (Chapman & Hall 1993). Also preferred is the use of positron-emitting radionuclides (PET isotopes), such as with an energy of 511 keV, such as ¹⁸F, ⁶⁸Ga, and ¹²⁴I. Such radionuclides may be imaged by well-known PET scanning techniques.

### Kits

Various embodiments may concern kits containing components suitable for imaging, diagnosing and/or detecting diseased tissue in a patient using labeled compounds. Exemplary kits may contain a targeting peptide of use as described herein.

A device capable of delivering the kit components may be included. One type of device, for applications such as parenteral delivery, is a syringe that is used to inject the composition into the body of a subject. Inhalation devices may also be used for certain applications.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

### EXAMPLES

### Example 1

### ¹⁸F-Labeling of Peptide IMP272

The first peptide that was prepared and ¹⁸F-labeled was IMP272:

(SEQ ID NO: 6)
DTPA-Gln-Ala-Lys(HSG)-D-Tyr-Lys(HSG)-NH₂

Acetate buffer solution—Acetic acid, 1.509 g was diluted in ˜160 mL water and the pH was adjusted by the addition of 1 M NaOH then diluted to 250 mL to make a 0.1 M solution at pH 4.03.

Aluminum acetate buffer solution—A solution of aluminum was prepared by dissolving 0.1028 g of AlCl₃ hexahydrate in 42.6 mL DI water. A 4 mL aliquot of the aluminum solution was mixed with 16 mL of a 0.1 M NaOAc solution at pH 4 to provide a 2 mM Al stock solution.

IMP272 acetate buffer solution—Peptide, 0.0011 g, 7.28×10⁻⁷ mol IMP272 was dissolved in 364 μL of the 0.1 M pH 4 acetate buffer solution to obtain a 2 mM stock solution of the peptide.

¹⁸F-Labeling of IMP272—A 3 μL aliquot of the aluminum stock solution was placed in a REACTI-VIAL™ and mixed with 50 μL ¹⁸F (as received) and 3 μL of the IMP272 solution. The solution was heated in a heating block at 110° C. for 15 min and analyzed by reverse phase HPLC. The HPLC trace (not shown) showed 93% free ¹⁸F and 7% bound to the peptide. An additional 10 μL of the IMP272 solution was added to the reaction and it was heated again and analyzed by reverse phase HPLC (not shown). The HPLC trace showed 8% ¹⁸F at the void volume and 92% of the activity attached to the peptide. The remainder of the peptide solution was incubated at room temperature with 150 μL PBS for ˜1 hr and then examined by reverse phase HPLC. The HPLC (not shown) showed 58% ¹⁸F unbound and 42% still attached to the peptide. The data indicate that Al¹⁸F(DTPA) complex may be unstable when mixed with phosphate.

### Example 2

### IMP272 ¹⁸F-Labeling with Other Metals

A ˜3 μL aliquot of the metal stock solution (6×10⁻⁹ mol) was placed in a polypropylene cone vial and mixed with 75 μL ¹⁸F (as received), incubated at room temperature for ˜2 min and then mixed with 20 μL of a 2 mM (4×10⁻⁸ mol) IMP272 solution in 0.1 M NaOAc pH 4 buffer. The solution was heated in a heating block at 100° C. for 15 min and analyzed by reverse phase HPLC. IMP272 was labeled with indium (24%), gallium (36%), zirconium (15%), lutetium (37%) and yttrium (2%) (not shown). These results demonstrate that the ¹⁸F metal labeling technique is not limited to an aluminum ligand, but can also utilize other metals as well. With different metal ligands, different chelating moieties may be utilized to optimize binding of an ¹⁸F-metal conjugate.

### Example 3

### Production and Use of a Serum-Stable ¹⁸F-Labeled Peptide IMP449

(SEQ ID NO: 7)
NOTA-benzyl-ITC-D-Ala-D-Lys(HSG)-D-Tyr-D-Lys(HSG)-
NH₂

The peptide, IMP448 D-Ala-D-Lys(HSG)-D-Tyr-D-Lys(HSG)-NH₂ (SEQ ID NO:8) was made on Sieber Amide resin by adding the following amino acids to the resin in the order shown: Aloc-D-Lys(Fmoc)-OH, Trt-HSG-OH, the Aloc was cleaved, Fmoc-D-Tyr(But)-OH, Aloc-D-Lys(Fmoc)-OH, Trt-HSG-OH, the Aloc was cleaved, Fmoc-D-Ala-OH with final Fmoc cleavage to make the desired peptide. The peptide was then cleaved from the resin and purified by HPLC to produce IMP448, which was then coupled to ITC-benzyl NOTA.

IMP448 (0.0757 g, 7.5×10⁻⁵ mol) was mixed with 0.0509 g (9.09×10⁻⁵ mol) ITC benzyl NOTA and dissolved in 1 mL water. Potassium carbonate anhydrous (0.2171 g) was then slowly added to the stirred peptide/NOTA solution. The reaction solution was pH 10.6 after the addition of all the carbonate. The reaction was allowed to stir at room temperature overnight. The reaction was carefully quenched with 1 M HCl after 14 hr and purified by HPLC to obtain 48 mg of IMP449.

¹⁸ F-Labeling of IMP449

IMP449 (0.002 g, 1.37×10⁻⁶ mol) was dissolved in 686 μL (2 mM peptide solution) 0.1 M NaOAc pH 4.02. Three microliters of a 2 mM solution of Al in a pH 4 acetate buffer was mixed with 15 μL, 1.3 mCi of ¹⁸F. The solution was then mixed with 20 μL of the 2 mM IMP449 solution and heated at 105° C. for 15 min. Reverse Phase HPLC analysis showed 35% (t_{R}˜10 min) of the activity was attached to the peptide and 65% of the activity was eluted at the void volume of the column (3.1 min, not shown) indicating that the majority of activity was not associated with the peptide. The crude labeled mixture (5 μL) was mixed with pooled human serum and incubated at 37° C. An aliquot was removed after 15 min and analyzed by HPLC. The HPLC showed 9.8% of the activity was still attached to the peptide (down from 35%). Another aliquot was removed after 1 hr and analyzed by HPLC. The HPLC showed 7.6% of the activity was still attached to the peptide (down from 35%), which was essentially the same as the 15 min trace (data not shown).

High Dose ^{˜}F-Labeling of IMP449

Further studies with purified IMP449 demonstrated that the ¹⁸F-labeled peptide was highly stable (91%, not shown) in human serum at 37° C. for at least one hour and was partially stable (76%, not shown) in human serum at 37° C. for at least four hours. Additional studies were performed in which the IMP449 was prepared in the presence of ascorbic acid as a stabilizing agent. In those studies (not shown), the ¹⁸F-metal-peptide complex showed no detectable decomposition in serum after 4 hr at 37° C. The mouse urine 30 min after injection of ¹⁸F-labeled peptide was found to contain ¹⁸F bound to the peptide (not shown). These results demonstrate that the ¹⁸F-labeled peptides disclosed herein exhibit sufficient stability under approximated in vivo conditions to be used for ¹⁸F imaging studies.

Since IMP449 peptide contains a thiourea linkage, which is sensitive to radiolysis, several products are observed by RP-HPLC. However, when ascorbic acid is added to the reaction mixture, the side products generated are markedly reduced.

### Example 4

### Preparation of DNL Constructs for ^{˜}F Imaging by Pretargeting

The DNL technique may be used to make dimers, trimers, tetramers, hexamers, etc. comprising virtually any antibodies or fragments thereof or other effector moieties. For certain preferred embodiments, IgG antibodies, Fab fragments or other proteins or peptides may be produced as fusion proteins containing either a DDD (dimerization and docking domain) or AD (anchoring domain) sequence. Bispecific antibodies may be formed by combining a Fab-DDD fusion protein of a first antibody with a Fab-AD fusion protein of a second antibody. Alternatively, constructs may be made that combine IgG-AD fusion proteins with Fab-DDD fusion proteins. For purposes of ¹⁸F detection, an antibody or fragment containing a binding site for an antigen associated with a target tissue to be imaged, such as a tumor, may be combined with a second antibody or fragment that binds a hapten on a targetable construct, such as IMP 449, to which a metal-¹⁸F can be attached. The bispecific antibody (DNL construct) is administered to a subject, circulating antibody is allowed to clear from the blood and localize to target tissue, and the ¹⁸F-labeled targetable construct is added and binds to the localized antibody for imaging.

Independent transgenic cell lines may be developed for each Fab or IgG fusion protein. Once produced, the modules can be purified if desired or maintained in the cell culture supernatant fluid. Following production, any DDD₂-fusion protein module can be combined with any corresponding AD-fusion protein module to generate a bispecific DNL construct. For different types of constructs, different AD or DDD sequences may be utilized. The following DDD sequences are based on the DDD moiety of PKA RIIα, while the AD sequences are based on the AD moiety of the optimized synthetic AKAP-IS sequence (Alto et al., Proc. Natl. Acad. Sci. USA. 2003; 100:4445).

DDD1:
(SEQ ID NO: 9)
SHIQIPPGLTELLQGYTVEVLRQQPPDLVEFAVEYFTRLREARA
DDD2:
(SEQ ID NO: 10)
CGHIQIPPGLTELLQGYTVEVLRQQPPDLVEFAVEYFTRLREARA
AD1:
(SEQ ID NO: 11)
QIEYLAKQIVDNAIQQA
AD2:
(SEQ ID NO: 12)
CGQIEYLAKQIVDNAIQQAGC

The plasmid vector pdHL2 has been used to produce a number of antibodies and antibody-based constructs. See Gillies et al., J Immunol Methods (1989), 125:191-202; Losman et al., Cancer (Phila) (1997), 80:2660-6. The di-cistronic mammalian expression vector directs the synthesis of the heavy and light chains of IgG. The vector sequences are mostly identical for many different IgG-pdHL2 constructs, with the only differences existing in the variable domain (VH and VL) sequences. Using molecular biology tools known to those skilled in the art, these IgG expression vectors can be converted into Fab-DDD or Fab-AD expression vectors. To generate Fab-DDD expression vectors, the coding sequences for the hinge, CH2 and CH3 domains of the heavy chain are replaced with a sequence encoding the first 4 residues of the hinge, a 14 residue Gly-Ser linker and the first 44 residues of human RIIα (referred to as DDD1). To generate Fab-AD expression vectors, the sequences for the hinge, CH2 and CH3 domains of IgG are replaced with a sequence encoding the first 4 residues of the hinge, a 15 residue Gly-Ser linker and a 17 residue synthetic AD called AKAP-IS (referred to as AD1), which was generated using bioinformatics and peptide array technology and shown to bind RIIα dimers with a very high affinity (0.4 nM). See Alto, et al. Proc. Natl. Acad. Sci., U.S.A (2003), 100:4445-50.

Two shuttle vectors were designed to facilitate the conversion of IgG-pdHL2 vectors to either Fab-DDD1 or Fab-AD1 expression vectors, as described below.

Preparation of CH1

The CH1 domain was amplified by PCR using the pdHL2 plasmid vector as a template. The left PCR primer consisted of the upstream (5′) end of the CH1 domain and a SacII restriction endonuclease site, which is 5′ of the CH1 coding sequence. The right primer consisted of the sequence coding for the first 4 residues of the hinge followed by four glycines and a serine (SEQ ID NO: 31), with the final two codons (GS) comprising a Bam HI restriction site. The 410 bp PCR amplimer was cloned into the pGemT PCR cloning vector (Promega, Inc.) and clones were screened for inserts in the T7 (5′) orientation.

A duplex oligonucleotide was synthesized by to code for the amino acid sequence of DDD1 preceded by 11 residues of a linker peptide, with the first two codons comprising a BamHI restriction site. A stop codon and an EagI restriction site are appended to the 3′ end. The encoded polypeptide sequence is shown below, with the DDD1 sequence underlined.

(SEQ ID NO: 13)
GSGGGGSGGGGSHIQIPPGLTELLQGYTVEVLRQQPPDLVEFAVEYFTRL
REARA

Two oligonucleotides, designated RIIA1-44 top and RIIA1-44 bottom, that overlap by 30 base pairs on their 3′ ends, were synthesized (Sigma Genosys) and combined to comprise the central 154 base pairs of the 174 by DDD1 sequence. The oligonucleotides were annealed and subjected to a primer extension reaction with Taq polymerase. Following primer extension, the duplex was amplified by PCR. The amplimer was cloned into pGemT and screened for inserts in the T7 (5′) orientation.

A duplex oligonucleotide was synthesized to code for the amino acid sequence of AD1 preceded by 11 residues of the linker peptide with the first two codons comprising a BamHI restriction site. A stop codon and an EagI restriction site are appended to the 3′end. The encoded polypeptide sequence is shown below, with the sequence of AD1 underlined.

(SEQ ID NO: 14)
GSGGGGSGGGGSQIEYLAKQIVDNAIQQA

Two complimentary overlapping oligonucleotides encoding the above peptide sequence, designated AKAP-IS Top and AKAP-IS Bottom, were synthesized and annealed. The duplex was amplified by PCR. The amplimer was cloned into the pGemT vector and screened for inserts in the T7 (5′) orientation.

Ligating DDD1 with CH1

A 190 bp fragment encoding the DDD1 sequence was excised from pGemT with BamHI and NotI restriction enzymes and then ligated into the same sites in CH1-pGemT to generate the shuttle vector CH1-DDD1-pGemT.

Ligating AD1 with CH1

A 110 bp fragment containing the AD1 sequence was excised from pGemT with BamHI and NotI and then ligated into the same sites in CH1-pGemT to generate the shuttle vector CH1-AD1-pGemT.

Cloning CH1-DDD1 or CH1-AD1 into pdHL2-Based Vectors

With this modular design either CH1-DDD1 or CH1-AD1 can be incorporated into any IgG construct in the pdHL2 vector. The entire heavy chain constant domain is replaced with one of the above constructs by removing the SacII/EagI restriction fragment (CH1-CH3) from pdHL2 and replacing it with the SacII/EagI fragment of CH1-DDD1 or CH1-AD1, which is excised from the respective pGemT shuttle vector.

Construction of h679-Fd-AD1-pdHL2

h679-Fd-AD1-pdHL2 is an expression vector for production of h679 Fab with AD1 coupled to the carboxyl terminal end of the CH1 domain of the Fd via a flexible Gly/Ser peptide spacer composed of 14 amino acid residues. A pdHL2-based vector containing the variable domains of h679 was converted to h679-Fd-AD1-pdHL2 by replacement of the SacII/EagI fragment with the CH1-AD1 fragment, which was excised from the CH1-AD1-SV3 shuttle vector with SacII and EagI.

Construction of C-DDD1-Fd-hMN-14-pdHL2

C-DDD1-Fd-hMN-14-pdHL2 is an expression vector for production of a stable dimer that comprises two copies of a fusion protein C-DDD1-Fab-hMN-14, in which DDD1 is linked to hMN-14 Fab at the carboxyl terminus of CH1 via a flexible peptide spacer. The plasmid vector hMN14(I)-pdHL2, which has been used to produce hMN-14 IgG, was converted to C-DDD1-Fd-hMN-14-pdHL2 by digestion with SacII and EagI restriction endonucleases to remove the CH1-CH3 domains and insertion of the CH1-DDD1 fragment, which was excised from the CH1-DDD1-SV3 shuttle vector with SacII and EagI.

The same technique has been utilized to produce plasmids for Fab expression of a wide variety of known antibodies, such as hLL1, hLL2, hPAM4, hRl, hRS7, hMN-14, hMN-15, hA19, hA20 and many others. Generally, the antibody variable region coding sequences were present in a pdHL2 expression vector and the expression vector was converted for production of an AD- or DDD-fusion protein as described above. The AD- and DDD-fusion proteins comprising a Fab fragment of any of such antibodies may be combined, in an approximate ratio of two DDD-fusion proteins per one AD-fusion protein, to generate a trimeric DNL construct comprising two Fab fragments of a first antibody and one Fab fragment of a second antibody.

C-DDD2-Fd-hMN-14-pdHL2

C-DDD2-Fd-hMN-14-pdHL2 is an expression vector for production of C-DDD2-Fab-hMN-14, which possesses a dimerization and docking domain sequence of DDD2 appended to the carboxyl terminus of the Fd of hMN-14 via a 14 amino acid residue Gly/Ser peptide linker. The fusion protein secreted is composed of two identical copies of hMN-14 Fab held together by non-covalent interaction of the DDD2 domains.

Two overlapping, complimentary oligonucleotides, which comprise the coding sequence for part of the linker peptide and residues 1-13 of DDD2, were made synthetically. The oligonucleotides were annealed and phosphorylated with T4 PNK, resulting in overhangs on the 5′ and 3′ ends that are compatible for ligation with DNA digested with the restriction endonucleases BamHI and PstI, respectively.

The duplex DNA was ligated with the shuttle vector CH1-DDD1-pGemT, which was prepared by digestion with BamHI and PstI, to generate the shuttle vector CH1-DDD2-pGemT. A 507 bp fragment was excised from CH1-DDD2-pGemT with SacII and EagI and ligated with the IgG expression vector hMN14(I)-pdHL2, which was prepared by digestion with SacII and EagI. The final expression construct was designated C-DDD2-Fd-hMN-14-pdHL2. Similar techniques have been utilized to generated DDD2-fusion proteins of the Fab fragments of a number of different humanized antibodies.

H679-Fd-AD2-pdHL2

h679-Fab-AD2, was designed to pair as B to C-DDD2-Fab-hMN-14 as A. h679-Fd-AD2-pdHL2 is an expression vector for the production of h679-Fab-AD2, which possesses an anchor domain sequence of AD2 appended to the carboxyl terminal end of the CH1 domain via a 14 amino acid residue Gly/Ser peptide linker AD2 has one cysteine residue preceding and another one following the anchor domain sequence of AD1.

The expression vector was engineered as follows. Two overlapping, complimentary oligonucleotides (AD2 Top and AD2 Bottom), which comprise the coding sequence for AD2 and part of the linker sequence, were made synthetically. The oligonucleotides were annealed and phosphorylated with T4 PNK, resulting in overhangs on the 5′ and 3′ ends that are compatible for ligation with DNA digested with the restriction endonucleases BamHI and SpeI, respectively.

The duplex DNA was ligated into the shuttle vector CH1-AD1-pGemT, which was prepared by digestion with BamHI and SpeI, to generate the shuttle vector CH1-AD2-pGemT. A 429 base pair fragment containing CH1 and AD2 coding sequences was excised from the shuttle vector with SacII and EagI restriction enzymes and ligated into h679-pdHL2 vector that prepared by digestion with those same enzymes. The final expression vector is h679-Fd-AD2-pdHL2.

### Example 5

### Generation of TF2 DNL Construct

A trimeric DNL construct designated TF2 was obtained by reacting C-DDD2-Fab-hMN-14 with h679-Fab-AD2. A pilot batch of TF2 was generated with >90% yield as follows. Protein L-purified C-DDD2-Fab-hMN-14 (200 mg) was mixed with h679-Fab-AD2 (60 mg) at a 1.4:1 molar ratio. The total protein concentration was 1.5 mg/ml in PBS containing 1 mM EDTA. Subsequent steps involved TCEP reduction, HIC chromatography, DMSO oxidation, and IMP 291 affinity chromatography. Before the addition of TCEP, SE-HPLC did not show any evidence of a₂b formation. Addition of 5 mM TCEP rapidly resulted in the formation of a₂b complex consistent with a 157 kDa protein expected for the binary structure. TF2 was purified to near homogeneity by IMP 291 affinity chromatography (not shown). IMP 291 is a synthetic peptide containing the HSG hapten to which the 679 Fab binds (Rossi et al., 2005, Clin Cancer Res 11:7122s-29s). SE-HPLC analysis of the IMP 291 unbound fraction demonstrated the removal of a₄, a₂ and free kappa chains from the product (not shown).

Non-reducing SDS-PAGE analysis demonstrated that the majority of TF2 exists as a large, covalent structure with a relative mobility near that of IgG (not shown). The additional bands suggest that disulfide formation is incomplete under the experimental conditions (not shown). Reducing SDS-PAGE shows that any additional bands apparent in the non-reducing gel are product-related (not shown), as only bands representing the constituent polypeptides of TF2 are evident. MALDI-TOF mass spectrometry (not shown) revealed a single peak of 156,434 Da, which is within 99.5% of the calculated mass (157,319 Da) of TF2.

The functionality of TF2 was determined by BIACORE assay. TF2, C-DDD1-hMN-14+h679-AD1 (used as a control sample of noncovalent a₂b complex), or C-DDD2-hMN-14+h679-AD2 (used as a control sample of unreduced a₂ and b components) were diluted to 1 μg/ml (total protein) and passed over a sensorchip immobilized with HSG. The response for TF2 was approximately two-fold that of the two control samples, indicating that only the h679-Fab-AD component in the control samples would bind to and remain on the sensorchip. Subsequent injections of WI2 IgG, an anti-idiotype antibody for hMN-14, demonstrated that only TF2 had a DDD-Fab-hMN-14 component that was tightly associated with h679-Fab-AD as indicated by an additional signal response. The additional increase of response units resulting from the binding of WI2 to TF2 immobilized on the sensorchip corresponded to two fully functional binding sites, each contributed by one subunit of C-DDD2-Fab-hMN-14. This was confirmed by the ability of TF2 to bind two Fab fragments of WI2 (not shown).

### Example 6

### Production of TF10 DNL Construct

A similar protocol was used to generate a trimeric TF10 DNL construct, comprising two copies of a C-DDD2-Fab-hPAM4 and one copy of C-AD2-Fab-679. The TF10 bispecific ([hPAM4]₂×h679) antibody was produced using the method disclosed for production of the (anti CEA)₂×anti HSG bsAb TF2, as described above. The TF10 construct bears two humanized PAM4 Fabs and one humanized 679 Fab.

The two fusion proteins (hPAM4-DDD2 and h679-AD2) were expressed independently in stably transfected myeloma cells. The tissue culture supernatant fluids were combined, resulting in a two-fold molar excess of hPAM4-DDD2. The reaction mixture was incubated at room temperature for 24 hours under mild reducing conditions using 1 mM reduced glutathione. Following reduction, the DNL reaction was completed by mild oxidation using 2 mM oxidized glutathione. TF10 was isolated by affinity chromatography using IMP 291-affigel resin, which binds with high specificity to the h679 Fab.

### Example 7

### In Vivo Imaging Using ¹⁸ F-Labeled Peptides and Comparison with ¹⁸F-FDG

In vivo imaging techniques using pretargeting with bispecific antibodies and labeled targeting peptides were used to successfully detect tumors of relatively small size. The ¹⁸F was purified on a WATERS® ACCELL™ Plus QMA Light cartridge. The ¹⁸F eluted with 0.4 M KHCO₃ was mixed with 3 μL 2 mM Al³⁺ in a pH 4 acetate buffer. The Al¹⁸F solution was then injected into the ascorbic acid IMP449 labeling vial and heated to 105° C. for 15 min. The reaction solution was cooled and mixed with 0.8 mL DI water. The reaction contents were loaded on a WATERS® OASIS® 1 cc HLB Column and eluted with 2×200 μL 1:1 EtOH/H₂O. TF2 was prepared as described above. TF2 binds divalently to carcinoembryonic antigen (CEA) and monovalently to the synthetic hapten, HSG (histamine-succinyl-glycine).

Biodistribution and microPET Imaging.

Six-week-old NCr nu-m female nude mice were implanted s.c. with the human colonic cancer cell line, LS174T (ATCC, Manassas, Va.). When tumors were visibly established, pretargeted animals were injected intravenously with 162 μg (−1 nmole/0.1 mL) TF2 or TF10 (control non-targeting tri-Fab bsMAb), and then 16-18 h later, ˜0.1 nmol of Al¹⁸F(IMP449) (84 μCi, 3.11 MBq/0.1 mL) was injected intravenously. Other non-pretargeted control animals received ¹⁸F alone (150 μCi, 5.5 MBq), Al¹⁸F complex alone (150 μCi, 5.55 MBq), the Al¹⁸F(IMP449) peptide alone (84 μCi, 3.11 MBq), or ¹⁸F-FDG (150 μCi, 5.55 MBq). ¹⁸F and ¹⁸F-FDG were obtained on the day of use from IBA Molecular (Somerset, N.J.). Animals receiving ¹⁸F-FDG were fasted overnight, but water was given ad libitum.

At 1.5 h after the radiotracer injection, animals were anesthetized, bled intracardially, and necropsied. Tissues were weighed and counted together with a standard dilution prepared from each of the respective products. Due to the short physical half-life of ¹⁸F, standards were interjected between each group of tissues from each animal. Uptake in the tissues is expressed as the counts per gram divided by the total injected activity to derive the percent-injected dose per gram (% ID/g).

Two types of imaging studies were performed. In one set, 3 nude mice bearing small LS174T subcutaneous tumors received either the pretargeted Al¹⁸F(IMP449), Al¹⁸F(IMP449) alone (not pretargeted), both at 135 μCi (5 MBq; 0.1 nmol), or ¹⁸F-FDG (135 μCi, 5 MBq). At 2 h after the intravenous radiotracer injection, the animals were anesthetized with a mixture of O₂/N₂O and isoflurane (2%) and kept warm during the scan, performed on an INVEON® animal PET scanner (Siemens Preclinical Solutions, Knoxville, Tenn.).

Representative coronal cross-sections (0.8 mm thick) in a plane located approximately in the center of the tumor were displayed, with intensities adjusted until pixel saturation occurred in any region of the body (excluding the bladder) and without background adjustment.

In a separate dynamic imaging study, a single LS174T bearing nude mouse that was given the TF2 bsMAb 16 h earlier was anesthetized with a mixture of O₂/N₂O and isoflurane (2%), placed supine on the camera bed, and then injected intravenously with 219 μCi (8.1 MBq) Al¹⁸F(IMP449) (0.16 nmol). Data acquisition was immediately initiated over a period of 120 minutes. The scans were reconstructed using OSEM3D/MAP. For presentation, time-frames ending at 5, 15, 30, 60, 90, and 120 min were displayed for each cross-section (coronal, sagittal, and transverse). For sections containing tumor, at each interval the image intensity was adjusted until pixel saturation first occurred in the tumor. Image intensity was increased as required over time to maintain pixel saturation within the tumor. Coronal and sagittal cross-sections without tumor taken at the same interval were adjusted to the same intensity as the transverse section containing the tumor. Background activity was not adjusted.

Results

While ¹⁸F alone and [Al¹⁸F] complexes had similar uptake in all tissues, considerable differences were found when the complex was chelated to IMP449 (Table 1). The most striking differences were found in the uptake in the bone, where the non-chelated ¹⁸F was 60- to nearly 100-fold higher in the scapula and ˜200-fold higher in the spine. This distribution is expected since ¹⁸F, or even a metal-fluoride complex, is known to accrete in bone (Franke et al. 1972, Radiobiol. Radiother. (Berlin) 13:533). Higher uptake was also observed in the tumor and intestines as well as in muscle and blood. The chelated Al¹⁸F(IMP449) had significantly lower uptake in all the tissues except the kidneys, illustrating the ability of the chelate-complex to be removed efficiently from the body by urinary excretion.

Pretargeting the Al¹⁸F(IMP449) using the TF2 anti-CEA bsMAb shifted uptake to the tumor, increasing it from 0.20±0.05 to 6.01±1.72% injected dose per gram at 1.5 h, while uptake in the normal tissues was similar to the Al¹⁸F(IMP449) alone. Tumor/nontumor ratios were 146±63, 59±24, 38±15, and 2.0±1.0 for the blood, liver, lung, and kidneys, respectively, with other tumor/tissue ratios >100:1 at this time. Although both ¹⁸F alone and [Al¹⁸F] alone had higher uptake in the tumor than the chelated Al¹⁸F(IMP449), yielding tumor/blood ratios of 6.7±2.7 and 11.0±4.6 vs. 5.1±1.5, respectively, tumor uptake and tumor/blood ratios were significantly increased with pretargeting (all P values<0.001).

Biodistribution was also compared to the most commonly used tumor imaging agent, [¹⁸F]FDG, which targets tissues with high glucose consumption and metabolic activity (Table 1). Its uptake was appreciably higher than the Al¹⁸F(IMP449) in all normal tissues, except the kidney. Tumor uptake was similar for both the pretargeted Al¹⁸F(IMP449) and ¹⁸F-FDG, but because of the higher accretion of [¹⁸F]FDG in most normal tissues, tumor/nontumor ratios with ¹⁸F-FDG were significantly lower than those in the pretargeted animals (all P values<0.001).

TABLE 1
Biodistribution of TF2-pretargeted Al¹⁸F(IMP449) and other control ¹⁸F-labeled agents in nude
mice bearing LS174T human colonic xenografts. For pretargeting, animals were given TF2 16 h before
the injection of the Al¹⁸F(IMP449). All injections were administered intravenously.

| | Percent Injected Dose Per Gram (Mean ± SD) at 1.5 hr Post-Injection | | | | |
|---|---|---|---|---|---|
| | | | Al¹⁸F(IMP449) | TF2-pretargeted | |
| | ¹⁸F alone | [Al¹⁸F] alone | alone | Al¹⁸F(IMP449) | ¹⁸F-FDG |
| Tumor | 1.02 ± 0.45 | 1.38 ± 0.39 | 0.20 ± 0.05 | 6.01 ± 1.72 | 7.25 ± 2.54 |
| Liver | 0.11 ± 0.02 | 0.12 ± 0.02 | 0.08 ± 0.03 | 0.11 ± 0.03 | 1.34 ± 0.36 |
| Spleen | 0.13 ± 0.06 | 0.10 ± 0.03 | 0.08 ± 0.02 | 0.08 ± 0.02 | 2.62 ± 0.73 |
| Kidney | 0.29 ± 0.07 | 0.25 ± 0.07 | 3.51 ± 0.56 | 3.44 ± 0.99 | 1.50 ± 0.61 |
| Lung | 0.26 ± 0.08 | 0.38 ± 0.19 | 0.11 ± 0.03 | 0.17 ± 0.04 | 3.72 ± 1.48 |
| Blood | 0.15 ± 0.03 | 0.13 ± 0.03 | 0.04 ± 0.01 | 0.04 ± 0.02 | 0.66 ± 0.19 |
| Stomach | 0.21 ± 0.13 | 0.15 ± 0.05 | 0.20 ± 0.32 | 0.12 ± 0.18 | 2.11 ± 1.04 |
| Small Int. | 1.53 ± 0.33 | 1.39 ± 0.34 | 0.36 ± 0.23 | 0.27 ± 0.10 | 1.77 ± 0.61 |
| Large Int. | 1.21 ± 0.13 | 1.78 ± 0.70 | 0.05 ± 0.04 | 0.03 ± 0.01 | 2.90 ± 0.79 |
| Scapula | 6.13 ± 1.33 | 9.83 ± 2.31 | 0.08 ± 0.06 | 0.04 ± 0.02 | 10.63 ± 5.88 |
| Spine | 19.88 ± 2.12 | 19.03 ± 2.70 | 0.13 ± 0.14 | 0.08 ± 0.03 | 4.21 ± 1.79 |
| Muscle | 0.16 ± 0.05 | 0.58 ± 0.36 | 0.06 ± 0.05 | 0.10 ± 0.20 | 4.35 ± 3.01 |
| Brain | 0.15 ± 0.06 | 0.13 ± 0.03 | 0.01 ± 0.01 | 0.01 ± 0.00 | 10.71 ± 4.53 |
| Tumor wt (g) | 0.29 ± 0.07 | 0.27 ± 0.10 | 0.27 ± 0.08 | 0.33 ± 0.11 | 0.25 ± 0.21 |
| N | 6 | 7 | 8 | 7 | 5 |

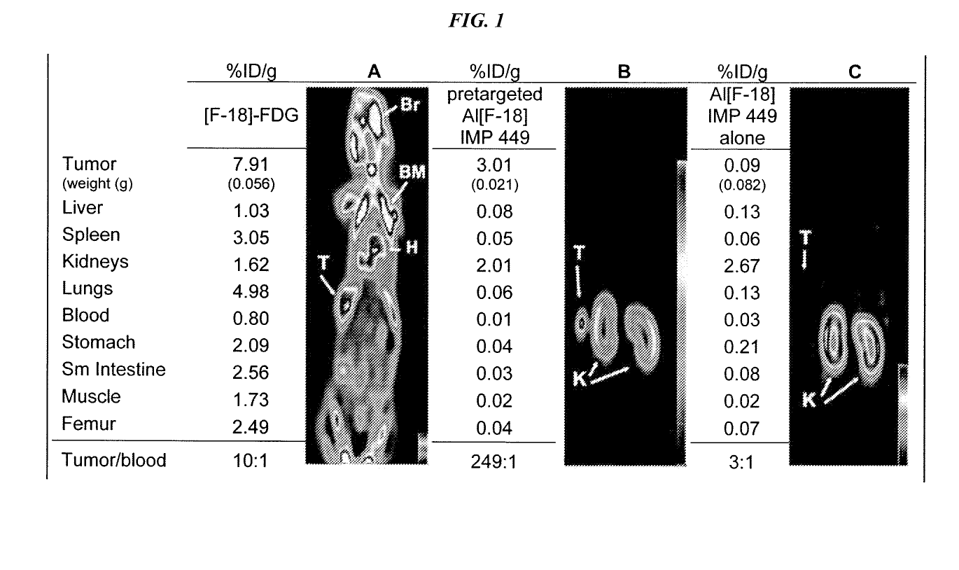

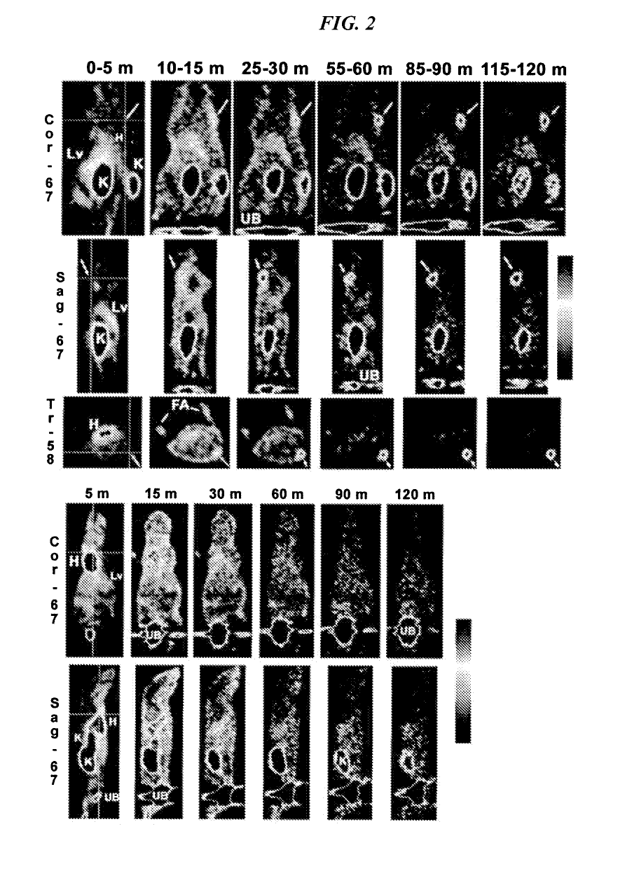

Several animals were imaged to further analyze the biodistribution of Al¹⁸F(IMP449) alone or Al¹⁸F(IMP449) pretargeted with TF2, as well [¹⁸F]FDG. Static images initiated at 2.0 h after the radioactivity was injected corroborated the previous tissue distribution data showing uptake almost exclusively in the kidneys (FIG. 1). A 21-mg tumor was easily visualized in the pretargeted animal, while the animal given the Al¹⁸F(IMP449) alone failed to localize the tumor, having only renal uptake. No evidence of bone accretion was observed, suggesting that the Al¹⁸F was bound firmly to IMP 449. This was confirmed in another pretargeted animal that underwent a dynamic imaging study that monitored the distribution of the Al¹⁸F(IMP449) in 5-min intervals over 120 minutes (FIG. 2). Coronal and sagittal slices showed primarily cardiac, renal, and some hepatic uptake over the first 5 min, but heart and liver activity decreased substantially over the next 10 min, while the kidneys remained prominent throughout the study. There was no evidence of activity in the intestines or bone over the full 120-min scan. Uptake in a 35-mg LS174T tumor was first observed at 15 min, and by 30 min, the signal was very clearly delineated from background, with intense tumor activity being prominent during the entire 120-min scanning

In comparison, static images from an animal given ¹⁸F-FDG showed the expected pattern of radioactivity in the bone, heart muscle, and brain observed previously (McBride et al., 2006, J. Nucl. Med. 47:1678; Sharkey et al., 2008, Radiology 246:497), with considerably more background activity in the body (FIG. 1). Tissue uptake measured in the 3 animals necropsied at the conclusion of the static imaging study confirmed much higher tissue ¹⁸F radioactivity in all tissues (not shown). While tumor uptake with ¹⁸F-FDG was higher in this animal than in the pretargeted one, tumor/blood ratios were more favorable for pretargeting; and with much less residual activity in the body, tumor visualization was enhanced by pretargeting.

These studies demonstrate that a hapten-peptide used in pretargeted imaging can be rapidly labeled (60 min total preparation time) with ¹⁸F by simply forming an aluminum-fluoride complex that can then be bound by a suitable chelate and incorporated into the hapten-peptide. This can be made more general by simply coupling the [Al¹⁸F]-chelate to any molecule that can be attached to the chelating moiety and be subsequently purified.

This report describes a direct, facile, and rapid method of binding ¹⁸F to various compounds via an aluminum conjugate. The [Al¹⁸F] peptide was stable in vitro and in vivo when bound by a NOTA-based chelate. Yields were within the range found with conventional ¹⁸F labeling procedures. These results further demonstrate the feasibility of PET imaging using metal ¹⁸F chelated to a wide variety of targeting molecules.

### Example 8

### Preparation and Labeling of IMP460 with Al-¹⁸F

IMP460 NOTA-Ga-D-Ala-D-Lys(HSG)-D-Tyr-D-Lys(HSG)-NH₂ (SEQ ID NO:15) was chemically synthesized. The NOTA-Ga ligand was purchased from CHEMATECH® and attached on the peptide synthesizer like the other amino acids. The peptide was synthesized on Sieber amide resin with the amino acids and other agents added in the following order Aloc-D-Lys(Fmoc)-OH, Trt-HSG-OH, Aloc removal, Fmoc-D-Tyr(But)-OH, Aloc-D-Lys(Fmoc)-OH, Trt-HSG-OH, Aloc removal, Fmoc-D-Ala-OH, and NOTA-GA(tBu)₃. The peptide was then cleaved and purified by HPLC to afford the product.

Radiolabeling of IMP460

IMP 460 (0.0020 g) was dissolved in 732 μL, pH 4, 0.1 M NaOAc. The ¹⁸F was purified as described above, neutralized with glacial acetic acid and mixed with the Al solution. The peptide solution, 20 μL was then added and the solution was heated at 99° C. for 25 min. The crude product was then purified on a WATERS® HLB column. The [Al¹⁸F] labeled peptide was in the 1:1 EtOH/H₂O column eluent. The reverse phase HPLC trace in 0.1% TFA buffers showed a clean single HPLC peak at the expected location for the labeled peptide (not shown).

### Example 9

### Synthesis and Labeling of IMP461 and IMP462 NOTA-Conjugated Peptides

The simplest possible NOTA ligand (protected for peptide synthesis) was prepared and incorporated into two peptides for pretargeting—IMP461 and IMP462.

Synthesis of Bis-t-butyl-NOTA

NO2AtBu (0.501 g 1.4×10⁻³ mol) was dissolved in 5 mL anhydrous acetonitrile. Benzyl-2-bromoacetate (0.222 mL, 1.4×10⁻³ mol) was added to the solution followed by 0.387 g of anhydrous K₂CO₃. The reaction was allowed to stir at room temperature overnight. The reaction mixture was filtered and concentrated to obtain 0.605 g (86% yield) of the benzyl ester conjugate. The crude product was then dissolved in 50 mL of isopropanol, mixed with 0.2 g of 10% Pd/C (under Ar) and placed under 50 psi H₂ for 3 days. The product was then filtered and concentrated under vacuum to obtain 0.462 g of the desired product ESMS [M−H]⁻ 415.

Synthesis of IMP461

The peptide was synthesized on Sieber amide resin with the amino acids and other agents added in the following order Aloc-D-Lys(Fmoc)-OH, Trt-HSG-OH, Aloc removal, Fmoc-D-Tyr(But)-OH, Aloc-D-Lys(Fmoc)-OH, Trt-HSG-OH, Aloc removal, Fmoc-D-Ala-OH, and Bis-t-butylNOTA. The peptide was then cleaved and purified by HPLC to afford the product IMP461 ESMS MH⁺ 1294 NOTA-D-Ala-D-Lys(HSG)-D-Tyr-D-Lys(HSG)-NH₂; SEQ ID NO:32).

Synthesis of IMP 462

The peptide was synthesized on Sieber amide resin with the amino acids and other agents added in the following order Aloc-D-Lys(Fmoc)-OH, Trt-HSG-OH, Aloc removal, Fmoc-D-Tyr(But)-OH, Aloc-D-Lys(Fmoc)-OH, Trt-HSG-OH, Aloc removal, Fmoc-D-Asp(But)-OH, and Bis-t-butyl NOTA. The peptide was then cleaved and purified by HPLC to afford the product IMP462 ESMS MH⁺ 1338 (NOTA-D-Asp-D-Lys(HSG)-D-Tyr-D-Lys(HSG)-NH₂; SEQ ID NO:16).

¹⁸F Labeling of IMP461 & IMP462

The peptides were dissolved in pH 4.13, 0.5 M NaOAc to make a 0.05 M peptide solution, which was stored in the freezer until needed. The F-18 was received in 2 mL of water and trapped on a SEP-PAK® Light, WATERS® ACCELL™ Plus QMA Cartridge. The ¹⁸F was eluted from the column with 200 μL aliquots of 0.4 M KHCO₃. The bicarbonate was neutralized to ˜pH 4 by the addition of 10 μL of glacial acetic acid to the vials before the addition of the activity. A 100 μL aliquot of the purified ¹⁸F solution was removed and mixed with 3 μL, 2 mM Al in pH 4, 0.1 M NaOAc. The peptide, 10 μL (0.05 M) was added and the solution was heated at ˜100° C. for 15 min. The crude reaction mixture was diluted with 700 μL DI water and placed on an HLB column and after washing the ¹⁸F was eluted with 2×100 μL of 1:1 EtOH/H₂O to obtain the purified ¹⁸F-labeled peptide.

### Example 10

### Preparation and ¹⁸ F-Labeling of IMP467

IMP467
(SEQ ID NO: 17)
C-NETA-succinyl-D-Lys(HSG)-D-Tyr-D-Lys(HSG)-NH₂

Tetra tert-butyl C-NETA-succinyl was produced. The tert-Butyl {4-[2-(Bis-(tert-butyoxycarbonyl)methyl-3-(4-nitrophenyl)propyl]-7-tert-butyoxycarbonyl[1,4,7]triazanonan-1-yl} was prepared as described in Chong et al. (J. Med. Chem. 2008, 51:118-125).

The peptide, IMP467 C-NETA-succinyl-D-Lys(HSG)-D-Tyr-D-Lys(HSG)-NH₂ (SEQ ID NO:17) was made on Sieber Amide resin by adding the following amino acids to the resin in the order shown: Aloc-D-Lys(Fmoc)-OH, Trt-HSG-OH, the Aloc was cleaved Fmoc-D-Tyr(But)-OH, Aloc-D-Lys(Fmoc)-OH, Trt-HSG-OH, the Aloc was cleaved, tert-Butyl{4-[Bis-(tert-butoxycarbonylmethyl)amino)-3-(4-succinylamidophenyl)propyl]-7-tert-butoxycarbonylmethyl[1,4,7]triazanonan-1-yl} acetate. The peptide was then cleaved from the resin and purified by RP-HPLC to yield 6.3 mg of IMP467. The crude peptide was purified by high performance liquid chromatography (HPLC) using a C18 column.

Radiolabeling

A 2 mM solution of IMP467 was prepared in pH 4, 0.1 M NaOAc. The ¹⁸F⁻, 139 mCi, was eluted through a WATERS® ACCELL™ Plus SEP-PAK® Light QMA cartridge and the ¹⁸F⁻ was eluted with 1 mL of 0.4 M KHCO₃.The labeled IMP467 was purified by HLB RP-HPLC. The RP-HPLC showed two peaks eluting (not shown), which are believed to be diastereomers of Al¹⁸F(IMP467). Supporting this hypothesis, there appeared to be some interconversion between the two HLB peaks when IMP467 was incubated at 37° C. (not shown). In pretargeting techniques as discussed below, since the [Al¹⁸F]-chelator complex is not part of the hapten site for antibody binding, the presence of diastereomers does not appear to affect targeting of the ¹⁸F-labeled peptide to diseased tissues.

Comparison of Yield of Radiolabeled Peptides

In an attempt to improve labeling yields while maintaining in vivo stability, 3 NOTA derivatives of pretargeting peptide were synthesized (IMP460, IMP461 and IMP467). Of these, IMP467 nearly doubled the labeling yields of the other peptides (Table 2). All of the labeling studies in Table 2 were performed with the same number of moles of peptide and aluminum. The results shown in Table 2 represent an exemplary labeling experiment with each peptide.

The ¹⁸F-labeling yield of IMP467 was ˜70% when only 40 nmol (˜13-fold less than IMP449) was used with 1.3 GBq (35 mCi) of ¹⁸F, indicating this ligand has improved binding properties for the Al¹⁸F complex. By enhancing the kinetics of ligand binding, yields were substantially improved (average 65-75% yield), while using fewer moles of IMP467 (40 nmol), relative to IMP449 (520 nmol, 44% yield).

TABLE 2
Comparison of yields of different NOTA containing peptides

| Peptide | Yield |
|---|---|
| IMP449 | 44% |
| IMP460 | 5.8% |
| IMP461 | 31% |
| IMP467 | 87% |

### Example 11

### Factors Affecting Yield and Stability of IMP467 Labeling

Peptide Concentration

To examine the effect of varying peptide concentration on yield, the amount of binding of Al¹⁸F to peptide was determined in a constant volume (63 μL) with a constant amount of Al³⁺ (6 nmol) and ¹⁸F, but varying the amount of peptide added. The yield of labeled peptide IMP467 decreased with a decreasing concentration of peptide as follows: 40 nmol peptide (82% yield); 30 nmol (79% yield); 20 nmol (75% yield); 10 nmol (49% yield). Thus, varying the amount of peptide between 20 and 40 nmol had little effect on yield with IMP467. However, a decreased yield was observed starting at 10 nmol of peptide in the labeling mix.

Aluminum Concentration

When IMP467 was labeled in the presence of increasing amounts of Al³⁺ (0, 5, 10, 15, 20 μL of 2 mM Al in pH 4 acetate buffer and keeping the total volume constant), yields of 3.5%, 80%, 77%, 78% and 74%, respectively, were achieved. These results indicated that (a) non-specific binding of ¹⁸F to this peptide in the absence of Al³⁺ is minimal, (b) 10 nmol of Al³⁺ was sufficient to allow for maximum ¹⁸F-binding, and (c) higher amounts of Al³⁺ did not reduce binding substantially, indicating that there was sufficient chelation capacity at this peptide concentration.

Kinetics of Al¹⁸F(IMP467) Radiolabeling

Kinetic studies showed that binding was complete within 5 min at 107° C. (5 min, 68%; 10 min, 61%; 15 min, 71%; and 30 min, 75%) with only moderate increases in isolated yield with reaction times as long as 30 min. A radiolabeling reaction of IMP467 performed at 50° C. showed that no binding was achieved at the lower temperature. Additional experiments, disclosed in the Examples below, show that under some conditions a limited amount of labeling can occur at reduced temperatures.

Effect of pH

The optimal pH for labeling was between 4.3 and 5.5. Yield ranged from 54% at pH 2.88; 70-77% at pH 3.99; 70% at pH 5; 41% at pH 6 to 3% at pH 7.3. The process could be expedited by eluting the ¹⁸F⁻ from the anion exchange column with nitrate or chloride ion instead of carbonate ion, which eliminates the need for adjusting the eluent to pH 4 with glacial acetic acid before mixing with the AlCl₃.

High-Dose Radiolabeling of IMP467

Five microliters of 2 mM Al³⁺ stock solution were mixed with 50 μL of ¹⁸F 1.3 GBq (35 mCi) followed by the addition of 20 μL of 2 mM IMP467 in 0.1 mM, pH 4.1 acetate buffer. The reaction solution was heated to 104° C. for 15 min and then purified on an HLB column (−10 min) as described above, isolating 0.68 GBq (18.4 mCi) of the purified peptide in 69% radiochemical yield with a specific activity of 17 GBq/μmol (460 Ci/mmol). The reaction time was 15 min and the purification time was 12 min. The reaction was started 10 min after the 1.3 GBq (35 mCi) ¹⁸F⁻ was purified, so the total time from the isolation of the ¹⁸F⁻ to the purified final product was 37 min with a 52% yield without correcting for decay.

Human Serum Stability Test

An aliquot of the HLB purified peptide (˜30 μL) was diluted with 200 μL human serum (previously frozen) and placed in the 37° C. HPLC sample chamber. Aliquots were removed at various time points and analyzed by HPLC. The HPLC analysis showed very high stability of the ¹⁸F-labeled peptides in serum at 37° C. for at least five hours (not shown). There was no detectable breakdown of the ¹⁸F-labeled peptide after a five hour incubation in serum (not shown).

The IMP461 and IMP462 ligands have two carboxyl groups available to bind the aluminum whereas the NOTA ligand in IMP467 had four carboxyl groups. The serum stability study showed that the complexes with IMP467 were stable in serum under conditions replicating in vivo use. In vivo biodistribution studies with labeled IMP467 show that the Al¹⁸F-labeled peptide is stable under actual in vivo conditions (not shown).

Peptides can be labeled with ¹⁸F rapidly (30 min) and in high yield by forming Al¹⁸F complexes that can be bound to a NOTA ligand on a peptide and at a specific activity of at least 17 GBq/μmol, without requiring HPLC purification. The Al¹⁸F(NOTA)-peptides are stable in serum and in vivo. Modifications of the NOTA ligand can lead to improvements in yield and specific activity, while still maintaining the desired in vivo stability of the Al¹⁸F(NOTA) complex, and being attached to a hydrophilic linker aids in the renal clearance of the peptide. Further, this method avoids the dry-down step commonly used to label peptides with ¹⁸F. As shown in the following Examples, this new ¹⁸F-labeling method is applicable to labeling of a broad spectrum of targeting peptides.

Optimized Labeling of Al¹⁸F(IMP467)

Optimized conditions for ¹⁸F-labeling of IMP467 were identified. These consisted of eluting ¹⁸F⁻ with commercial sterile saline (pH 5-7), mixing with 20 nmol of AlCl₃ and 40 nmol IMP467 in pH 4 acetate buffer in a total volume of 100 μL, heating to 102° C. for 15 min, and performing SPE separation. High-yield (85%) and high specific activity (115 GBq/μmol) were obtained with IMP467 in a single step, 30-min procedure after a simple solid-phase extraction (SPE) separation without the need for HPLC purification. Al¹⁸F(IMP467) was stable in PBS or human serum, with 2% loss of ¹⁸F⁻ after incubation in either medium for 6 h at 37° C.

Concentration and Purification of ¹⁸F⁻

Radiochemical-grade ¹⁸F⁻ needs to be purified and concentrated before use. We examined 4 different SPE purification procedures to process the ¹⁸F⁻ prior to its use. Most of the radiolabeling procedures were performed using ¹⁸F prepared by a conventional process. The ¹⁸F⁻ in 2 mL of water was loaded onto a SEP-PAK® Light, Waters Accell™ QMA Plus Cartridge that was pre-washed with 10 mL of 0.4M KHCO₃, followed by 10 mL water. After loading the ¹⁸F⁻ onto the cartridge, it was washed with 5 mL water to remove any dissolved metal and radiometal impurities. The isotope was then eluted with ˜1 mL of 0.4M KHCO₃ in several fractions to isolate the fraction with the highest concentration of activity. The eluted fractions were neutralized with 5 μL of glacial acetic acid per 100 μL of solution to adjust the eluent to pH 4-5.

In the second process, the QMA cartridge was washed with 10 mL pH 8.4, 0.5 M NaOAc followed by 10 mL DI H₂O. ¹⁸F⁻ was loaded onto the column as described above and eluted with 1 mL, pH 6, 0.05 M KNO₃ in 200-μL fractions with 60-70% of the activity in one of the fractions. No pH adjustment of this solution was needed.

In the third process, the QMA cartridge was washed with 10 mL pH 8.4, 0.5 M NaOAc followed by 10 mL DI H₂O. The ¹⁸F⁻ was loaded onto the column as described above and eluted with 1 mL, pH 5-7, 0.154 M commercial normal saline in 200-μL fractions with 80% of the activity in one of the fractions. No pH adjustment of this solution was needed.

Finally, we devised a method to prepare a more concentrated and high-activity ¹⁸F⁻ solution, using tandem ion exchange. Briefly, Tygon tubing (1.27 cm long, 0.64 cm OD) was inserted into a TRICORN™ 5/20 column and filled with ˜200 μL of AG 1-X8 resin, 100-200 mesh. The resin was washed with 6 mL 0.4 M K₂CO₃ followed by 6 mL H₂O. A SEP-PAK® light Waters ACCELL™ Plus CM cartridge was washed with DI H₂O. Using a syringe pump, the crude ¹⁸F⁻ that was received in 5-mL syringe in 2 mL DI H₂O flowed slowly through the CM cartridge and the TRICORN™ column over ˜5 min followed by a 6 mL wash with DI H₂O through both ion-binding columns. Finally, 0.4 M K₂CO₃ was pushed through only the TRICORN™ column in 50-μL fractions. Typically, 40 to 60% of the eluted activity was in one 50-μL fraction. The fractions were collected in 2.0 mL free-standing screw-cap microcentrifuge tubes containing 5 μL glacial acetic acid to neutralize the carbonate solution. The elution vial with the most activity was then used as the reaction vial.

### Example 12

### Labeling by Addition of ¹⁸F⁻ to a Peptide Complexed with Aluminum

An HSG containing peptide (IMP 465, Al(NOTA)-D-Ala-D-Lys(HSG)-D-Tyr-D-Lys(HSG)-NH₂) (SEQ ID NO:18) linked to macrocyclic NOTA complexed with aluminum, was successfully labeled with F-18. ¹⁸F incorporation using 40 nmol of IMP 465 was 13.20%. An intermediate peptide, IMP 461, was made as described above. Then 25.7 mg of IMP461 was dissolved in 2 mL DI water to which was added 10.2 mg AlCl₃3H₂O and the resultant solution heated to 100° C. for 1 h. The crude reaction mixture was purified by RP-HPLC to yield 19.6 mg of IMP465.

For ¹⁸F-labeling, 50 μL ¹⁸F solution [0.702 mCi of ¹⁸F⁻] and 20 μL (40 nmol) 2 mM IMP465 solution (0.1 M NaOAc, pH 4.18) was heated to 101° C. for 17 minutes. Reverse Phase HPLC analysis showed 15.38% (RT about 8.60 min) of the activity was attached to the peptide and 84.62% of the activity eluted at the void volume of the column (2.60 min).

In a separate experiment, the percent yield of ¹⁸F-labeled peptide could be improved by varying the amount of peptide added. The percent yield observed for IMP465 was 0.27% at 10 nmol peptide, 1.8% at 20 nmol of peptide and 49% at 40 nmol of peptide.

IMP467 showed higher yield than IMP461 when peptide was pre-incubated with aluminum before exposure to ¹⁸F. IMP467 was incubated with aluminum at room temperature and then frozen and lyophilized. The amount of aluminum added for the pre-incubation was varied.

TABLE 3
Labeling of IMP467 Pre-Incubated
with Aluminum Before ¹⁸F⁻ is Added

| | Isolated |
|---|---|
| IMP467 + Al Premixed, Frozen and Lyophilized | Labeling Yield |
| 40 nmol IMP467 + 10 nmol Al Premix | 82% |
| 40 nmol IMP467 + 20 nmol Al Premix | 64% |
| 40 nmol IMP467 + 30 nmol Al Premix | 74% |
| 40 nmol IMP467 + 6 nmol Al Normal Labeling | 77% |
| (Mix Al + ¹⁸F first) | |

The yields were comparable to those obtained when IMP467 is labeled by addition of an Al¹⁸F complex. Thus, ¹⁸F labeling by addition of ¹⁸F to a peptide with aluminum already bound to the chelating moiety is a feasible alternative approach to pre-incubating the metal with ¹⁸F⁻ prior to addition to the chelating moiety.

### Example 13

### Synthesis and Labeling of IMP468 Bombesin Peptide

The ¹⁸F labeled targeting moieties are not limited to antibodies or antibody fragments, but rather can include any molecule that binds specifically or selectively to a cellular target that is associated with or diagnostic of a disease state or other condition that may be imaged by ¹⁸F PET. Bombesin is a 14 amino acid peptide that is homologous to neuromedin B and gastrin releasing peptide, as well as a tumor marker for cancers such as lung and gastric cancer and neuroblastoma. IMP468 (NOTA-NH-(CH₂)₇CO-Gln-Trp-Val-Trp-Ala-Val-Gly-His-Leu-Met-NH₂; SEQ ID NO:19) was synthesized as a bombesin analogue and labeled with ¹⁸F to target the gastrin-releasing peptide receptor.

The peptide was synthesized by Fmoc based solid phase peptide synthesis on Sieber amide resin, using a variation of a synthetic scheme reported in the literature (Prasanphanich et al., 2007, PNAS USA 104:12463-467). The synthesis was different in that a bis-t-butyl NOTA ligand was add to the peptide during peptide synthesis on the resin.

IMP468 (0.0139 g, 1.02×10⁻⁵ mol) was dissolved in 203 μL of 0.5 M pH 4.13 NaOAc buffer. The peptide dissolved but formed a gel on standing so the peptide gel was diluted with 609 μL of 0.5 M pH 4.13 NaOAc buffer and 406 μL of ethanol to produce an 8.35×10⁻³ M solution of the peptide. The ¹⁸F was purified on a QMA cartridge and eluted with 0.4 M KHCO₃ in 200 μL fractions, neutralized with 10 μL of glacial acetic acid. The purified ¹⁸F, 40 μL, 1.13 mCi was mixed with 3 μL of 2 mM AlCl₃ in pH 4, 0.1 M NaOAc buffer. IMP468 (59.2 μL, 4.94 x 10⁻⁷ mol) was added to the Al¹⁸F solution and placed in a 108° C. heating block for 15 min. The crude product was purified on an HLB column, eluted with 2×200 μL of 1:1 EtOH/H₂O to obtain the purified ¹⁸F-labeled peptide in 34% yield.

### Example 14

### Imaging of Tumors Using ¹⁸F Labeled Bombesin

A NOTA-conjugated bombesin derivative (IMP468) was prepared as described above. We began testing its ability to block radiolabeled bombesin from binding to PC-3 cells as was done by Prasanphanich et al. (PNAS 104:12462-12467, 2007). Our initial experiment was to determine if IMP468 could specifically block bombesin from binding to PC-3 cells. We used IMP333 as a non-specific control. In this experiment, 3×10⁶ PC-3 cells were exposed to a constant amount (˜50,000 cpms) of ¹²⁵I-Bombesin (Perkin-Elmer) to which increasing amounts of either IMP468 or IMP333 was added. A range of 56 to 0.44 nM was used as our inhibitory concentrations.

The results showed that we could block the binding of ¹²⁵I-BBN with IMP468 but not with the control peptide (IMP333) (not shown), thus demonstrating the specificity of IMP468. Prasanphanich indicated an IC₅₀ for their peptide at 3.2 nM, which is approximately 7-fold lower than what we found with IMP468 (21.5 nM).

This experiment was repeated using a commercially available BBN peptide. We increased the amount of inhibitory peptide from 250 to 2 nM to block the ¹²⁵I-BBN from binding to PC-3 cells. We observed very similar IC₅₀-values for IMP468 and the BBN positive control with an IC₅₀-value higher (35.9 nM) than what was reported previously (3.2 nM) but close to what the BBN control achieved (24.4 nM).

To examine in vivo targeting, the distribution of Al¹⁸F(IMP468) was examined in scPC3 prostate cancer xenograft bearing nude male mice; alone vs. blocked with bombesin. For radiolabeling, aluminum chloride (10 μL, 2 mM), 51.9 mCi of ¹⁸F (from QMA cartridge), acetic acid, and 60 μL of IMP468 (8.45 mM in ethanol/NaOAc) were heated at 100° C. for 15 min. The reaction mixture was purified on reverse phase HPLC. Fractions 40 and 41 (3.56, 1.91 mCi) were pooled and applied to HLB column for solvent exchange. The product was eluted in 800 μL (3.98 mCi) and 910 μCi remained on the column. iTLC developed in saturated NaCl showed 0.1% unbound activity.

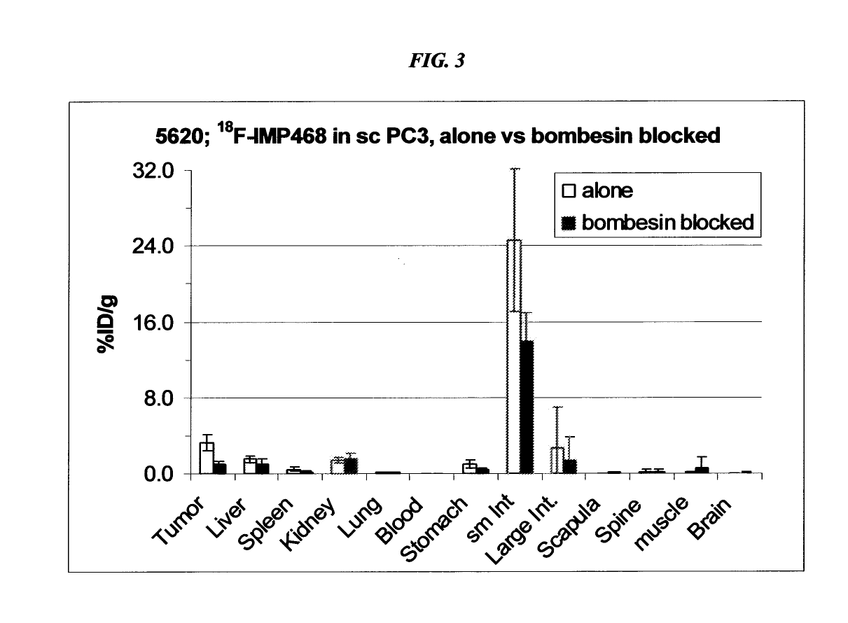

A group of six tumor-bearing mice were injected with Al¹⁸F(IMP468) (167 μCi, ˜9×10⁻¹⁰ mol) and necropsied 1.5 h later. Another group of six mice were injected iv with 100 μg (6.2×10⁻⁸ mol) of bombesin 18 min before administering Al¹⁸F(IMP468). The second group was also necropsied 1.5 h post injection. The data shows specific targeting of the tumor with [Al¹⁸F] IMP 468 (FIG. 3). Tumor uptake of the peptide is reduced when bombesin was given 18 min before the Al¹⁸F(IMP468) (FIG. 3). Biodistribution data indicates in vivo stability of Al¹⁸F(IMP468) for at least 1.5 h (not shown).

Larger tumors showed higher uptake of Al¹⁸F(IMP468), possibly due to higher receptor expression in larger tumors (not shown). The biodistribution data showed Al¹⁸F(IMP468) tumor targeting that was in the same range as reported for the same peptide labeled with ⁶⁸Ga by Prasanphanich et al. (not shown). The results demonstrate that the ¹⁸F peptide labeling method can be used in vivo to target receptors that are upregulated in tumors, using targeting molecules besides antibodies. In this case, the IMP468 targeting took advantage of a naturally occurring ligand-receptor interaction. The tumor targeting was significant with a P value of P=0.0013. Many such ligand-receptor pairs are known and any such targeting interaction may form the basis for ¹⁸F imaging, using the methods described herein.

### Example 15

### Synthesis and Labeling of Somatostatin Analog IMP466

Somatostatin is another non-antibody targeting peptide that is of use for imaging the distribution of somatostatin receptor protein. ¹²³I-labeled octreotide, a somatostatin analog, has been used for imaging of somatostatin receptor expressing tumors (e.g., Kvols et al., 1993, Radiology 187:129-33; Leitha et al., 1993, J Nucl Med 34:1397-1402). However, ¹²³I has not been of extensive use for imaging because of its expense, short physical half-life and the difficulty of preparing the radiolabeled compounds. The ¹⁸F-labeling methods described herein are preferred for imaging of somatostatin receptor expressing tumors.

IMP466
(SEQ ID NO: 20)
NOTA-D-Phe-Cys-Phe-D-Trp-Lys-Thr-Cys-Throl

A NOTA-conjugated derivative of the somatostatin analog octreotide (IMP466) was made by standard Fmoc based solid phase peptide synthesis to produce a linear peptide. The C-terminal Throl residue is threoninol. The peptide was cyclized by treatment with DMSO overnight. The peptide, 0.0073 g, 5.59×10⁻⁶ mol was dissolved in 111.9 μL of 0.5 M pH 4 NaOAc buffer to make a 0.05 M solution of IMP466. The solution formed a gel over time so it was diluted to 0.0125 M by the addition of more 0.5 M NaOAc buffer.

¹⁸F was purified and concentrated with a QMA cartridge to provide 200 μL of ¹⁸F in 0.4 M KHCO₃. The bicarbonate solution was neutralized with 10 μL of glacial acetic acid. A 40 μL aliquot of the neutralized ¹⁸F eluent was mixed with 3 μL of 2 mM AlCl₃, followed by the addition of 40 μL of 0.0125 M IMP466 solution. The mixture was heated at 105° C. for 17 min. The reaction was then purified on a Waters 1 cc (30 mg) HLB column by loading the reaction solution onto the column and washing the unbound ¹⁸F away with water (3 mL) and then eluting the radiolabeled peptide with 2×200 μL 1:1 EtOH water. The yield of the radiolabeled peptide after HLB purification was 34.6%.

### Effect of Ionic Strength

To lower the ionic strength of the reaction mixture escalating amounts of acetonitrile were added to the labeling mixture (final concentration: 0-80%). The yield of radiolabeled IMP466 increased with increasing concentration of acetonitrile in the medium. The optimal radiolabeling yield (98%) was obtained in a final concentration of 80% acetonitrile, despite the increased volume (500 μL in 80% vs. 200 μL in 0% acetonitrile). In 0% acetonitrile the radiolabeling yield ranged from 36% to 55% in three experiments.

### Example 16

### Imaging of Neuroendocrine Tumors with an ¹⁸F- and ⁶⁸Ga-Labeled IMP466

Studies were performed to compare the PET images obtained using an ¹⁸F versus ⁶⁸Ga-labeled somatostatin analogue peptide and direct targeting to somatostatin receptor expressing tumors.

### Methods

¹⁸F labeling—IMP466 was synthesized and ¹⁸F-labeled by a variation of the method described in the Example above. A QMA SEPPAK® light cartridge (Waters, Milford, Mass.) with 2-6 GBq ¹⁸F⁻ (BV Cyclotron VU, Amsterdam, The Netherlands) was washed with 3 mL metal-free water. ¹⁸F⁻ was eluted from the cartridge with 0.4 M KHCO₃ and fractions of 200 μL were collected. The pH of the fractions was adjusted to pH 4, with 10 μL metal-free glacial acid. Three μL of 2 mM AlCl₃ in 0.1 M sodium acetate buffer, pH 4 were added. Then, 10-50 μL IMP 466 (10 mg/mL) were added in 0.5 M sodium acetate, pH 4.1. The reaction mixture was incubated at 100° C. for 15 min unless stated otherwise. The radiolabeled peptide was purified on RP-HPLC. The Al¹⁸F(IMP466) containing fractions were collected and diluted two-fold with H₂O and purified on a 1-cc Oasis HLB cartridge (Waters, Milford, Mass.) to remove acetonitrile and TFA. In brief, the fraction was applied on the cartridge and the cartridge was washed with 3 mL H₂O. The radiolabeled peptide was then eluted with 2×200 μL 50% ethanol. For injection in mice, the peptide was diluted with 0.9% NaCl. A maximum specific activity of 45,000 GBq/mmol was obtained.

⁶⁸Ga labeling—IMP466 was labeled with ⁶⁸GaCl₃ eluted from a TiO₂-based 1,110 MBq ⁶⁸Ge/⁶⁸Ga generator (Cyclotron Co. Ltd., Obninsk, Russia) using 0.1 M ultrapure HCl (J. T. Baker, Deventer, The Netherlands). IMP466 was dissolved in 1.0 M HEPES buffer, pH 7.0. Four volumes of ⁶⁸Ga eluate (120-240 MBq) were added and the mixture was heated at 95° C. for 20 min. Then 50 mM EDTA was added to a final concentration of 5 mM to complex the non-incorporated ⁶⁸Ga³⁺. The ⁶⁸Ga-labeled IMP466 was purified on an Oasis HLB cartridge and eluted with 50% ethanol.

Octanol-water partition coefficient (log P_{oct/water})—To determine the lipophilicity of the radiolabeled peptides, approximately 50,000 dpm of the radiolabeled peptide was diluted in 0.5 mL phosphate-buffered saline (PBS). An equal volume of octanol was added to obtain a binary phase system. After vortexing the system for 2 min, the two layers were separated by centrifugation (100×g, 5 min). Three 100 μL samples were taken from each layer and radioactivity was measured in a well-type gamma counter (Wallac Wizard 3″, Perkin-Elmer, Waltham, Mass.).

Stability—Ten μL of the ¹⁸F-labeled IMP466 was incubated in 500 μL of freshly collected human serum and incubated for 4 h at 37° C. Acetonitrile was added and the mixture was vortexed followed by centrifugation at 1000×g for 5 min to precipitate serum proteins. The supernatant was analyzed on RP-HPLC as described above.

Cell culture—The AR42J rat pancreatic tumor cell line was cultured in Dulbecco's Modified Eagle's Medium (DMEM) medium (Gibco Life Technologies, Gaithersburg, Md., USA) supplemented with 4500 mg/L D-glucose, 10% (v/v) fetal calf serum, 2 mmol/L glutamine, 100 U/mL penicillin and 100 μg/mL streptomycin. Cells were cultured at 37° C. in a humidified atmosphere with 5% CO₂.

IC₅₀ determination—The apparent 50% inhibitory concentration (IC₅₀) for binding the somatostatin receptors on AR42J cells was determined in a competitive binding assay using Al¹⁹F(IMP466), ⁶⁹Ga(IMP466) or ¹¹⁵In(DTPA-octreotide) to compete for the binding of ¹¹¹In(DTPA-octreotide).

Al¹⁹F(IMP466) was formed by mixing an aluminium fluoride (Al¹⁹F) solution (0.02 M AlCl₃ in 0.5 M NaAc, pH 4, with 0.1 M NaF in 0.5 M NaAc, pH 4) with IMP466 and heating at 100° C. for 15 min. The reaction mixture was purified by RP-HPLC on a C-18 column as described above.

⁶⁹Ga(IMP466) was prepared by dissolving gallium nitrate (2.3×10⁻⁸ mol) in 30 μL mixed with 20 μL IMP466 (1 mg/mL) in 10 mM NaAc, pH 5.5, and heated at 90° C. for 15 min. Samples of the mixture were used without further purification.

¹¹⁵In(DTPA-octreotide) was made by mixing indium chloride (1×10⁻⁵ mol) with 10 μL DTPA-octreotide (1 mg/mL) in 50 mM NaAc, pH 5.5, and incubated at room temperature (RT) for 15 min. This sample was used without further purification. ¹¹¹In(DTPA-octreotide) (OCTREOSCAN®) was radiolabeled according to the manufacturer's protocol.

AR42J cells were grown to confluency in 12-well plates and washed twice with binding buffer (DMEM with 0.5% bovine serum albumin). After 10 min incubation at RT in binding buffer, Al¹⁹F(IMP466), ⁶⁹Ga(IMP466) or ¹¹⁵In(DTPA-octreotide) was added at a final concentration ranging from 0.1-1000 nM, together with a trace amount (10,000 cpm) of ¹¹¹In(DTPA-octreotide) (radiochemical purity>95%). After incubation at RT for 3 h, the cells were washed twice with ice-cold PBS. Cells were scraped and cell-associated radioactivity was determined. Under these conditions, a limited extent of internalization may occur. We therefore describe the results of this competitive binding assay as “apparent IC₅₀” values rather than IC₅ₒ. The apparent IC₅₀ was defined as the peptide concentration at which 50% of binding without competitor was reached.

Biodistribution studies—Male nude BALB/c mice (6-8 weeks) were injected subcutaneously in the right flank with 0.2 mL AR42J cell suspension of 10⁷ cells/mL. Approximately two weeks after tumor cell inoculation when tumors were 5-8 mm in diameter, 370 kBq ¹⁸F or ⁶⁸Ga-labeled IMP466 was administered intravenously (n=5). Separate groups (n=5) were injected with a 1,000-fold molar excess of unlabeled IMP466. One group of three mice was injected with unchelated [Al¹⁸F]. All mice were killed by CO₂/O₂ asphyxiation 2 h post-injection (p.i.). Organs of interest were collected, weighed and counted in a gamma counter. The percentage of the injected dose per gram tissue (%ID/g) was calculated for each tissue. The animal experiments were approved by the local animal welfare committee and performed according to national regulations.

PET/CT imaging—Mice with s.c. AR42J tumors were injected intravenously with 10 MBq Al¹⁸F(IMP466) or ⁶⁸Ga(IMP466). One and two hours after the injection of peptide, mice were scanned on an Inveon animal PET/CT scanner (Siemens Preclinical Solutions, Knoxville, Tenn.) with an intrinsic spatial resolution of 1.5 mm (Visser et al, JNM, 2009). The animals were placed in a supine position in the scanner. PET emission scans were acquired over 15 minutes, followed by a CT scan for anatomical reference (spatial resolution 113 μm, 80 kV, 500 μA). Scans were reconstructed using Inveon Acquisition Workplace software version 1.2 (Siemens Preclinical Solutions, Knoxville, TN) using an ordered set expectation maximization-3D/maximum a posteriori (OSEM3D/MAP) algorithm with the following parameters: matrix 256×256×159, pixel size 0.43×0.43×0.8 mm³ and MAP prior of 0.5 mm.

### Results

Effect of buffer—The effect of the buffer on the labeling efficiency of IMP466 was investigated. IMP466 was dissolved in sodium citrate buffer, sodium acetate buffer, 2-(N-morpholino)ethanesulfonic acid (MES) or 4-(2-hydroxyethyl)-1-piperazineethanesulfonic acid (HEPES) buffer at 10 mg/mL (7.7 mM). The molarity of all buffers was 1 M and the pH was 4.1. To 200 μg (153 nmol) of IMP466 was added 100 μL [Al¹⁸F] (pH 4) and incubated at 100° C. for 15 min. Radiolabeling yield and specific activity was determined with RP-HPLC. When using sodium acetate, MES or HEPES, radiolabeling yield was 49%, 44% and 46%, respectively. In the presence of sodium citrate, no labeling was observed (<1%). When the labeling reaction was carried out in sodium acetate buffer, the specific activity of the preparations was 10,000 GBq/mmol, whereas in MES and HEPES buffer a specific activity of 20,500 and 16,500 GBq/mmol was obtained, respectively.

Effect of AlCl₃ concentration—Three stock solutions of AlCl₃ in sodium acetate, pH 4.1 were prepared: 0.2, 2.0 and 20 mM. From these solutions, 3 μL was added to 200 μL of ¹⁸F⁻ to form [Al¹⁸F]. To these samples, 153 nmol of peptide was added and incubated for 15 min at 100° C. Radiolabeling yield was 49% after incubation at a final concentration of 6 nmol AlCl₃. Incubation with 0.6 nmol AlCl₃ and 60 nmol AlCl₃ resulted in a strong reduction of the radiolabeling yield: 10% and 6%, respectively.

Effect of amount of peptide—The effect of the amount of peptide on the labeling efficiency was investigated. IMP466 was dissolved in sodium acetate buffer, pH 4.1 at a concentration of 7.7 mM (10 mg/mL) and 38, 153 or 363 nmol of IMP466 was added to 200 μL [Al¹⁸F] (581-603 MBq). The radiolabeling yield increased with increasing amounts of peptide. At 38 nmol, radiolabeling yield ranged from 4-8%, at 153 nmol, the yield had increased to 42-49% and at the highest concentration the radiolabeling yield was 48-52%.

Octanol-water partition coefficient—To determine the lipophilicity of the ¹⁸F and ⁶⁸Ga-labeled IMP466, the octanol-water partition coefficients were determined. The log P_{octanol/water} value for the Al¹⁸F(IMP466) was −2.44±0.12 and that of ⁶⁸Ga(IMP466) was −3.79±0.07, indicating that the ¹⁸F-labeled IMP 466 was slightly less hydrophilic.

Stability—The ¹⁸F-labeled IMP466 did not show release of ¹⁸F after incubation in human serum at 37° C. for 4 h, indicating excellent stability of the Al[¹⁸F]NOTA complex.

IC₅₀ determination—The apparent IC₅₀ of Al¹⁹F(IMP466) was 3.6±0.6 nM, whereas that for ⁶⁹Ga(IMP466) was 13±3 nM. The apparent IC₅₀ of the reference peptide, ¹¹⁵In(DTPA-octeotride) (OCTREOSCAN®), was 6.3±0.9 nM.

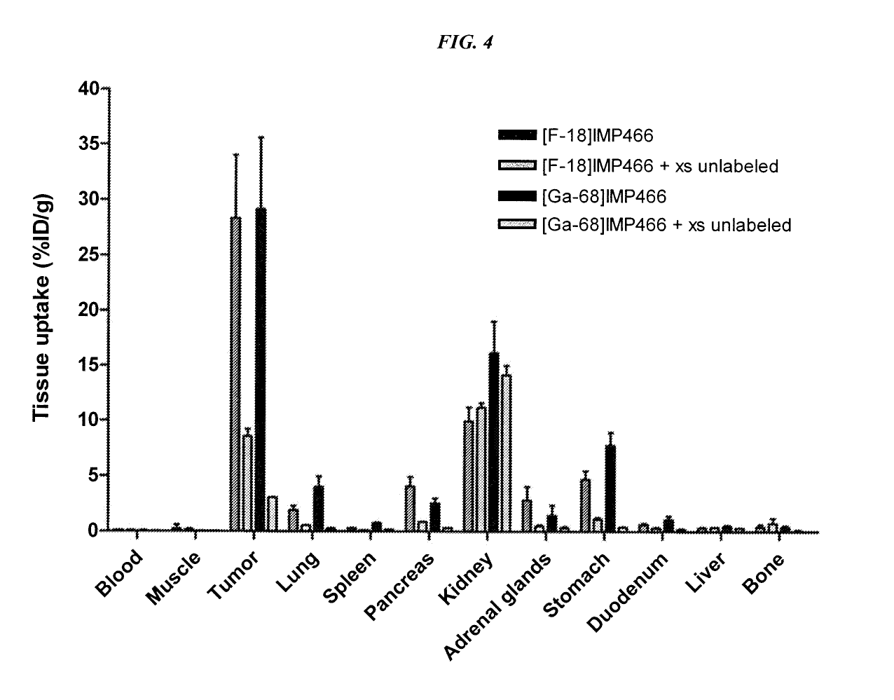

Biodistribution studies—The biodistribution of both Al¹⁸F(IMP466) and ⁶⁸Ga(IMP466) was studied in nude BALB/c mice with s.c. AR42J tumors at 2 h p.i. (FIG. 4). Al¹⁸F was included as a control. Tumor targeting of the Al¹⁸F(IMP466) was high with 28.3±5.7% ID/g accumulated at 2 h p.i. Uptake in the presence of an excess of unlabeled IMP466 was 8.6±0.7% ID/g, indicating that tumor uptake was receptor-mediated. Blood levels were very low (0.10±0.07%ID/g, 2 h pi), resulting in a tumor-to-blood ratio of 299±88. Uptake in the organs was low, with specific uptake in receptor expressing organs such as adrenal glands, pancreas and stomach. Bone uptake of Al¹⁸F(IMP466) was negligible as compared to uptake of non-chelated Al¹⁸F (0.33±0.07 vs. 36.9±5.0%ID/g at 2 h p.i., respectively), indicating good in vivo stability of the ¹⁸F-labeled NOTA-peptide.

The biodistribution of Al¹⁸F(IMP466) was compared to that of ⁶⁸Ga(IMP466) (FIG. 4). Tumor uptake of ⁶⁸Ga(IMP466) (29.2±0.5% ID/g, 2 h pi) was similar to that of Al¹⁸F-IMP 466 (p<0.001). Lung uptake of ⁶⁸Ga(IMP466) was two-fold higher than that of Al¹⁸F(IMP466) (4.0±0.9% ID/g vs. 1.9±0.4% ID/g, respectively). In addition, kidney retention of ⁶⁸Ga(IMP466) was slightly higher than that of Al¹⁸F(IMP466) (16.2±2.86%ID/g vs. 9.96±1.27% ID/g, respectively.

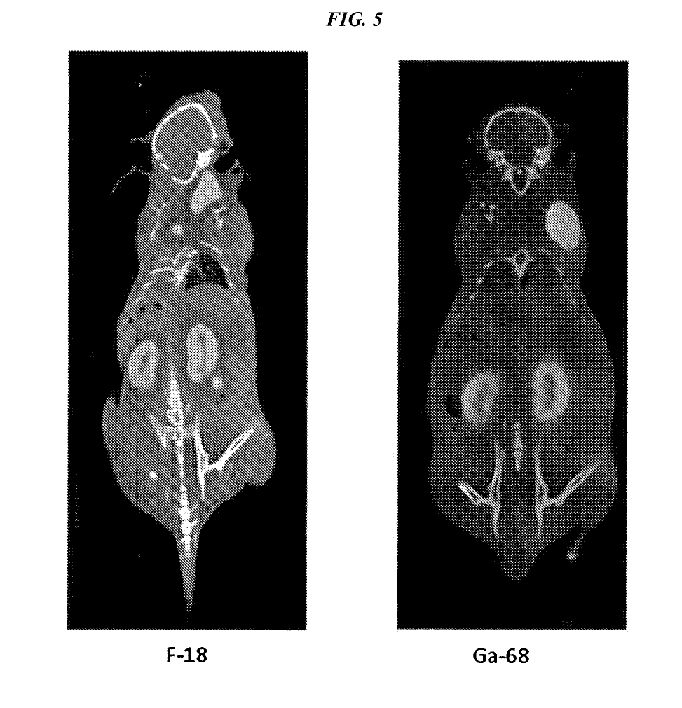

Fused PET and CT scans are shown in FIG. 5. PET scans corroborated the biodistribution data. Both Al¹⁸F(IMP466) and ⁶⁸Ga(IMP466) showed high uptake in the tumor and retention in the kidneys. The activity in the kidneys was mainly localized in the renal cortex. Again, the [Al¹⁸F] proved to be stably chelated by the NOTA chelator, since no bone uptake was observed.

FIG. 5 clearly shows that the distribution of an ¹⁸F-labeled analog of somatostatin (octreotide) mimics that of a ⁶⁸Ga-labeled somatostatin analog. These results are significant, since ⁶⁸Ga-labeled octreotide PET imaging in human subjects with neuroendocrine tumors has been shown to have a significantly higher detection rate compared with conventional somatostatin receptor scintigraphy and diagnostic CT, with a sensitivity of 97%, a specificity of 92% and an accuracy of 96% (e.g., Gabriel et al., 2007, J Nucl Med 48:508-18). PET imaging with ⁶⁸Ga-labeled octreotide is reported to be superior to SPECT analysis with ¹¹¹ In-labeled octreotide and to be highly sensitive for detection of even small meningiomas (Henze et al., 2001, J Nucl Med 42:1053-56). Because of the higher energy of ⁶⁸Ga compared with ¹⁸F, it is expected that ¹⁸F based PET imaging would show even better spatial resolution than ⁶⁸Ga based PET imaging. This is illustrated in FIG. 5 by comparing the kidney images obtained with ¹⁸F-labeled IMP466 (FIG. 5, left) vs. ⁶⁸Ga-labeled IMP466 (FIG. 5, right). The PET images obtained with ⁶⁸Ga show more diffuse margins and lower resolution than the images obtained with ¹⁸F. These results demonstrate the superior images obtained with ¹⁸F-labeled targeting moieties prepared using the methods and compositions described herein and confirm the utility of the described ¹⁸F-labeling techniques for non-antibody targeting peptides.

### Example 17

### Comparison of ⁶⁸Ga and ¹⁸F PET Imaging Using Pretargeting

We compared PET images obtained using ⁶⁸Ga- or ¹⁸F-labeled peptides that were pretargeted with the bispecific TF2 antibody, prepared as described above. The half-lives of ⁶⁸Ga (t_{1/2}=68 minutes) and ¹⁸F (t_{1/2}=110 minutes) match with the pharmacokinetics of the radiolabeled peptide, since its maximum accretion in the tumor is reached within hours. Moreover, ⁶⁸Ga is readily available from ⁶⁸Ge/⁶⁸Ga generators, whereas ¹⁸F is the most commonly used and widely available radionuclide in PET.

### Methods

Mice with s.c. CEA-expressing LS174T tumors received TF2 (6.0 nmol; 0.94 mg) and 5 MBq ⁶⁸Ga(IMP288) (0.25 nmol) or Al¹⁸F(IMP449) (0.25 nmol) intravenously, with an interval of 16 hours between the injection of the bispecific antibody and the radiolabeled peptide. One or two hours after the injection of the radiolabeled peptide, PET/CT images were acquired and the biodistribution of the radiolabeled peptide was determined. Uptake in the LS174T tumor was compared with that in an s.c. CEA-negative SK-RC 52 tumor. Pretargeted immunoPET imaging was compared with ¹⁸F-FDG PET imaging in mice with an s.c. LS174T tumor and contralaterally an inflamed thigh muscle.

IMP288
(SEQ ID NO: 21)
DOTA-D-Tyr-D-Lys(HSG)-D-Glu-D-Lys(HSG)-NH₂

Pretargeting—The bispecific TF2 antibody was made by the DNL method, as described above. TF2 is a trivalent bispecific antibody comprising an HSG-binding Fab fragment from the h679 antibody and two CEA-binding Fab fragments from the hMN-14 antibody. The DOTA-conjugated, HSG-containing peptide IMP288 was synthesized by peptide synthesis as described above. The IMP449 peptide, synthesized as described above, contains a 1,4,7-triazacyclononane-1,4,7-triacetic acid (NOTA) chelating moiety to facilitate labeling with ¹⁸F. As a tracer for the antibody component, TF2 was labeled with ¹²⁵1 (Perkin Elmer, Waltham, Mass.) by the iodogen method (Fraker and Speck, 1978, Biochem Biophys Res Comm 80:849-57), to a specific activity of 58 MBq/nmol.

Labeling of IMP288—IMP288 was labeled with ¹¹¹In (Covidien, Petten, The Netherlands) for biodistribution studies at a specific activity of 32 MBq/nmol under strict metal-free conditions. IMP288 was labeled with ⁶⁸Ga eluted from a TiO-based 1,110 MBq ⁶⁸Ge/⁶⁸Ga generator (Cyclotron Co. Ltd., Obninsk Russia) using 0.1 M ultrapure HC1. Five 1 ml fractions were collected and the second fraction was used for labeling the peptide. One volume of 1.0 M HEPES buffer, pH 7.0 was added to 3.4 nmol IMP 288. Four volumes of ⁶⁸Ga eluate (380 MBq) were added and the mixture was heated to 95° C. for 20 min. Then 50 mM EDTA was added to a final concentration of 5 mM to complex the non-chelated ⁶⁸Ga³⁺. The ⁶⁸Ga(IMP288) peptide was purified on a 1-mL Oasis HLB-cartridge (Waters, Milford, Mass.). After washing the cartridge with water, the peptide was eluted with 25% ethanol. The procedure to label IMP288 with ⁶⁸Ga was performed within 45 minutes, with the preparations being ready for in vivo use.

Labeling of IMP449—IMP449 was labeled with ¹⁸F as described above. 555-740 MBq ¹⁸F (B.V. Cyclotron VU, Amsterdam, The Netherlands) was eluted from a QMA cartridge with 0.4 M KHCO₃. The Al¹⁸F activity was added to a vial containing the peptide (230 μg) and ascorbic acid (10 mg). The mixture was incubated at 100° C. for 15 min. The reaction mixture was purified by RP-HPLC. After adding one volume of water, the peptide was purified on a 1-mL Oasis HLB cartridge. After washing with water, the radiolabeled peptide was eluted with 50% ethanol. Al¹⁸F(IMP449) was prepared within 60 minutes, with the preparations being ready for in vivo use.

Radiochemical purity of ¹²⁵I-TF2, ¹¹¹In(IMP288) and ⁶⁸Ga(IMP288) and Al¹⁸F(IMP449) preparations used in the studies always exceeded 95%.

Animal experiments—Experiments were performed in male nude BALB/c mice (6-8 weeks old), weighing 20-25 grams. Mice received a subcutaneous injection with 0.2 mL of a suspension of 1×10⁶ LS174T-cells, a CEA-expressing human colon carcinoma cell line (American Type Culture Collection, Rockville, MD, USA). Studies were initiated when the tumors reached a size of about 0.1-0.3 g (10-14 days after tumor inoculation).

The interval between TF2 and IMP288 injection was 16 hours, as this period was sufficient to clear unbound TF2 from the circulation. In some studies ¹²⁵I-TF2, (0.4 MBq) was co-injected with unlabeled TF2. IMP288 was labeled with either ¹¹¹ In or ⁶⁸Ga. IMP449 was labeled with ¹⁸F. Mice received TF2 and IMP288 intravenously (0.2 mL). One hour after the injection of ⁶⁸Ga-labeled peptide, and two hours after injection of Al¹⁸F(IMP449), mice were euthanized by CO₂ /O₂, and blood was obtained by cardiac puncture and tissues were dissected.

PET images were acquired with an Inveon animal PET/CT scanner (Siemens Preclinical Solutions, Knoxville, Tn.). PET emission scans were acquired for 15 minutes, preceded by CT scans for anatomical reference (spatial resolution 113 μm, 80 kV, 500 μA, exposure time 300 msec).

After imaging, tumor and organs of interest were dissected, weighed and counted in a gamma counter with appropriate energy windows for ¹²⁵I, ¹¹¹In, ⁶⁸or 18F. The percentage-injected dose per gram tissue (% ID/g) was calculated.

### Results

Within 1 hour, pretargeted immunoPET resulted in high and specific targeting of ⁶⁸Ga-IMP288 in the tumor (10.7±3.6% ID/g), with very low uptake in the normal tissues (e.g., tumor/blood 69.9±32.3), in a CEA-negative tumor (0.35±0.35% ID/g), and inflamed muscle (0.72±0.20% ID/g). Tumors that were not pretargeted with TF2 also had low ⁶⁸Ga(IMP288) uptake (0.20±0.03% ID/g). [¹⁸F]FDG accreted efficiently in the tumor (7.42±0.20% ID/g), but also in the inflamed muscle (4.07±1.13% ID/g) and a number of normal tissues, and thus pretargeted ⁶⁸Ga-IMP 288 provided better specificity and sensitivity. The corresponding PET/CT images of mice that received ⁶⁸Ga(IMP288) or Al¹⁸F(IMP449) following pretargeting with TF2 clearly showed the efficient targeting of the radiolabeled peptide in the subcutaneous LS174T tumor, while the inflamed muscle was not visualized. In contrast, with ¹⁸F-FDG the tumor as well as the inflammation was clearly delineated.

Dose optimization—The effect of the TF2 bsMAb dose on tumor targeting of a fixed 0.01 nmol (15 ng) dose of IMP288 was determined. Groups of five mice were injected intravenously with 0.10, 0.25, 0.50 or 1.0 nmol TF2 (16, 40, 80 or 160 μg respectively), labeled with a trace amount of ¹²⁵I (0.4 MBq). One hour after injection of ¹¹¹In(IMP288) (0.01 nmol, 0.4 MBq), the biodistribution of the radiolabels was determined.

TF2 cleared rapidly from the blood and the normal tissues. Eighteen hours after injection the blood concentration was less than 0.45% ID/g at all TF2 doses tested. Targeting of TF2 in the tumor was 3.5% ID/g at 2 h p.i. and independent of TF2 dose (data not shown). At all TF2 doses ¹¹¹In(IMP288) accumulated effectively in the tumor (not shown). At higher TF2 doses enhanced uptake of ¹¹¹In(IMP288) in the tumor was observed: at 1.0 nmol TF2 dose maximum targeting of IMP288 was reached (26.2±3.8% ID/g). Thus at the 0.01 nmol peptide dose highest tumor targeting and tumor-to-blood ratios were reached at the highest TF2 dose of 1.0 nmol (TF2:IMP288 molar ratio=100:1). Among the normal tissues, the kidneys had the highest uptake of ¹¹¹In(IMP288) (1.75±0.27% ID/g) and uptake in the kidneys was not affected by the TF2 dose (not shown). All other normal tissues had very low uptake, resulting in extremely high tumor-to-nontumor ratios, exceeding 50:1 at all TF2 doses tested (not shown).

For PET imaging using ⁶⁸Ga-labeled IMP288, a higher peptide dose is required, because a minimum activity of 5-10 MBq ⁶⁸Ga needs to be injected per mouse if PET imaging is performed 1 h after injection. The specific activity of the ⁶⁸Ga(IMP288) preparations was 50-125 MBq/nmol at the time of injection. Therefore, for PET imaging at least 0.1-0.25 nmol of IMP288 had to be administered. The same TF2:IMP288 molar ratios were tested at 0.1 nmol IMP288 dose. LS174T tumors were pretargeted by injecting 1.0, 2.5, 5.0 or 10.0 nmol TF2 (160, 400, 800 or 1600 μg). In contrast to the results at the lower peptide dose, ¹¹¹In(IMP288) uptake in the tumor was not affected by the TF2 doses (15% ID/g at all doses tested, data not shown). TF2 targeting in the tumor in terms of % ID/g decreased at higher doses (3.21±0.61% ID/g versus 1.16±0.27% ID/g at an injected dose of 1.0 nmol and 10.0 nmol, respectively) (data not shown). Kidney uptake was also independent of the bsMAb dose (2% ID/g). Based on these data we selected a bsMAb dose of 6.0 nmol for targeting 0.1-0.25 nmol of IMP288 to the tumor.

PET imaging—To demonstrate the effectiveness of pretargeted immunoPET imaging with TF2 and ⁶⁸Ga(IMP288) to image CEA-expressing tumors, subcutaneous tumors were induced in five mice. In the right flank an s.c. LS174T tumor was induced, while at the same time in the same mice 1×10⁶ SK-RC 52 cells were inoculated in the left flank to induce a CEA-negative tumor. Fourteen days later, when tumors had a size of 0.1-0.2 g, the mice were pretargeted with 6.0 nmol ¹²⁵I-TF2 intravenously. After 16 hours the mice received 5 MBq ⁶⁸Ga(IMP288) (0.25 nmol, specific activity of 20 MBq/nmol). A separate group of three mice received the same amount of ⁶⁸Ga-IMP 288 alone, without pretargeting with TF2. PET/CT scans of the mice were acquired 1 h after injection of the ⁶⁸Ga(IMP288).

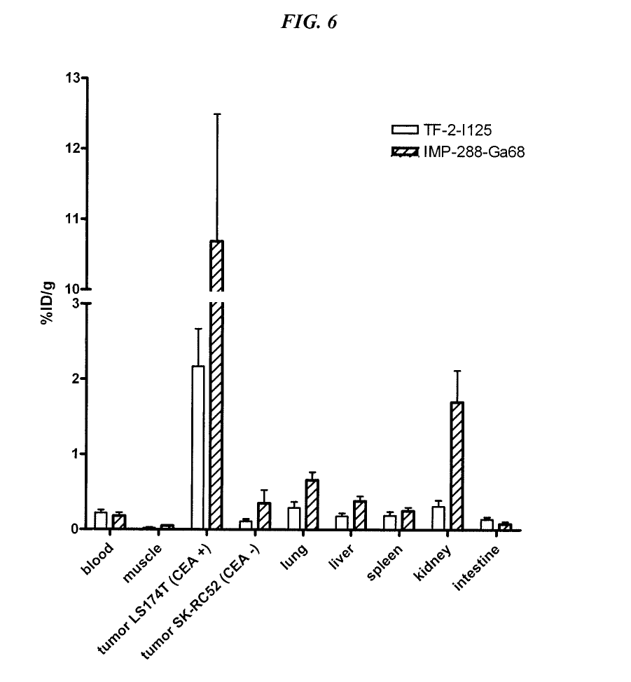

The biodistribution of ¹²⁵I-TF2 and [⁶⁸Ga]IMP288 in the mice are shown in FIG. 6. Again high uptake of the bsMAb (2.17±0.50% ID/g) and peptide (10.7±3.6% ID/g) in the tumor was observed, with very low uptake in the normal tissues (tumor-to-blood ratio: 64±22). Targeting of ⁶⁸Ga(IMP288) in the CEA-negative tumor SK-RC 52 was very low (0.35±0.35% ID/g). Likewise, tumors that were not pretargeted with TF2 had low uptake of ⁶⁸Ga(IMP288) (0.20±0.03% ID/g), indicating the specific accumulation of IMP288 in the CEA-expressing LS174T tumor.

The specific uptake of ⁶⁸Ga(IMP288) in the CEA-expressing tumor pretargeted with TF2 was clearly visualized in a PET image acquired 1 h after injection of the ⁶⁸Ga-labeled peptide (not shown). Uptake in the tumor was evaluated quantitatively by drawing regions of interest (ROI), using a 50% threshold of maximum intensity. A region in the abdomen was used as background region. The tumor-to-background ratio in the image of the mouse that received TF2 and ⁶⁸Ga(IMP288) was 38.2.

We then examined pretargeted immunoPET with ¹⁸F-FDG. In two groups of five mice a s.c. LS174T tumor was induced on the right hind leg and an inflammatory focus in the left thigh muscle was induced by intramuscular injection of 50 μL turpentine (18). Three days after injection of the turpentine, one group of mice received 6.0 nmol TF2, followed 16 h later by 5 MBq ⁶⁸Ga(IMP288) (0.25 nmol). The other group received ¹⁸F-FDG (5 MBq). Mice were fasted during 10 hours prior to the injection and anaesthetized and kept warm at 37° C. until euthanasia, 1 h postinjection.

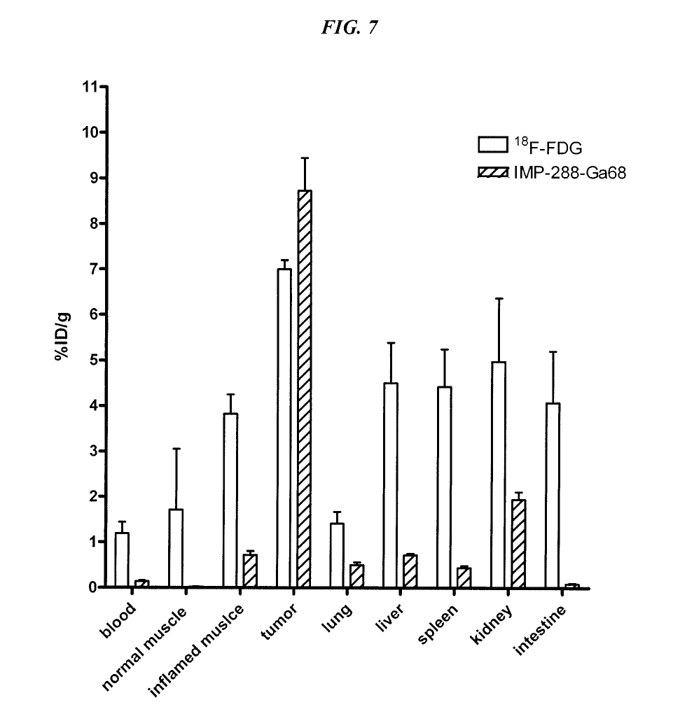

Uptake of ⁶⁸Ga(IMP288) in the inflamed muscle was very low, while uptake in the tumor in the same animal was high (0.72±0.20% ID/g versus 8.73±1.60% ID/g, p<0.05, FIG. 7). Uptake in the inflamed muscle was in the same range as uptake in the lungs, liver and spleen (0.50±0.14% ID/g, 0.72±0.07% ID/g, 0.44±0.10% ID/g, respectively). Tumor-to-blood ratio of ⁶⁸Ga(IMP288) in these mice was 69.9±32.3; inflamed muscle-to-blood ratio was 5.9 ±2.9; tumor-to-inflamed muscle ratio was 12.5±2.1. In the other group of mice ¹⁸F-FDG accreted efficiently in the tumor (7.42±0.20% ID/g, tumor-to-blood ratio 6.24±1.5, FIG. 4). ¹⁸F-FDG also substantially accumulated in the inflamed muscle (4.07±1.13% ID/g), with inflamed muscle-to-blood ratio of 3.4±0.5, and tumor-to-inflamed muscle ratio of 1.97±0.71.

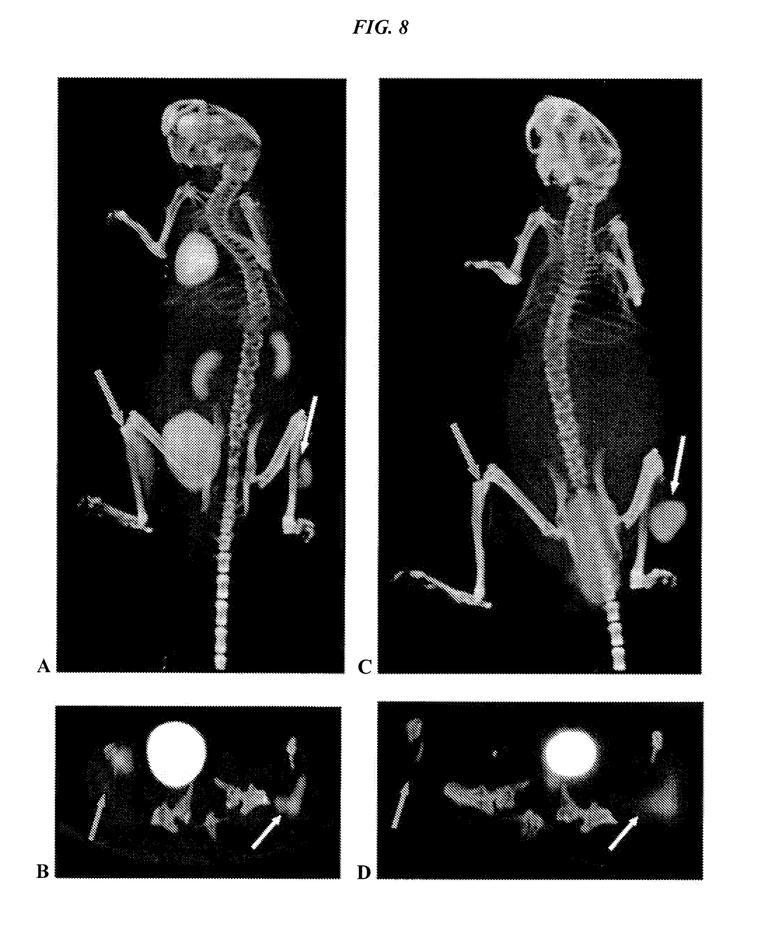

The corresponding PET/CT image of a mouse that received ⁶⁸Ga(IMP288), following pretargeting with TF2, clearly showed the efficient accretion of the radiolabeled peptide in the tumor, while the inflamed muscle was not visualized (FIG. 8). In contrast, on the images of the mice that received ¹⁸F-FDG, the tumor as well as the inflammation was visible (FIG. 8). In the mice that received ⁶⁸Ga(IMP288), the tumor-to-inflamed tissue ratio was 5.4; tumor-to-background ratio was 48; inflamed muscle-to-background ratio was 8.9. ¹⁸F-FDG uptake had a tumor-to-inflamed muscle ratio of 0.83; tumor-to-background ratio was 2.4; inflamed muscle-to-background ratio was 2.9.

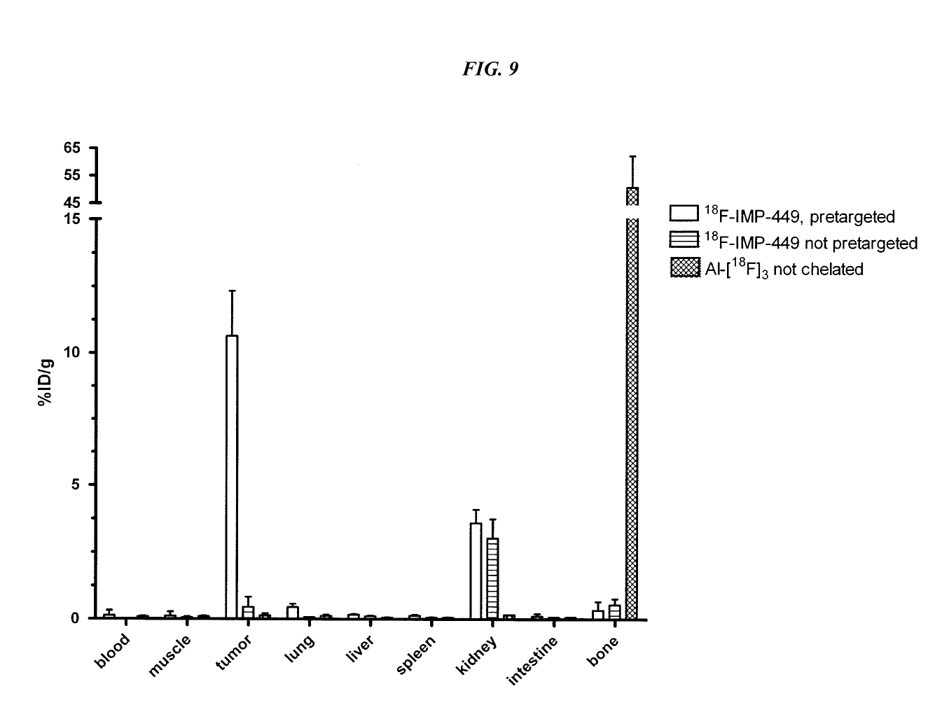

The pretargeted immunoPET imaging method was tested using the Al¹⁸F(IMP449). Five mice received 6.0 nmol TF2, followed 16 h later by 5 MBq Al[¹⁸F]IMP449 (0.25 nmol). Three additional mice received 5 MBq Al¹⁸F(IMP449) without prior administration of TF2, while two control mice were injected with [Al¹⁸F] (3 MBq). The results of this experiment are summarized in FIG. 9. Uptake of Al¹⁸F(IMP449) in tumors pretargeted with TF2 was high (10.6±1.7% ID/g), whereas it was very low in the non-pretargeted mice (0.45±0.38%ID/g). [Al¹⁸F] accumulated in the bone (50.9±11.4%ID/g), while uptake of the radiolabeled IMP449 peptide in the bone was very low (0.54±0.2% ID/g), indicating that the Al¹⁸F(IMP449) was stable in vivo. The biodistribution of Al¹⁸F(IMP449) in the TF2 pretargeted mice with s.c. LS174T tumors were highly similar to that of ⁶⁸Ga(IMP288).

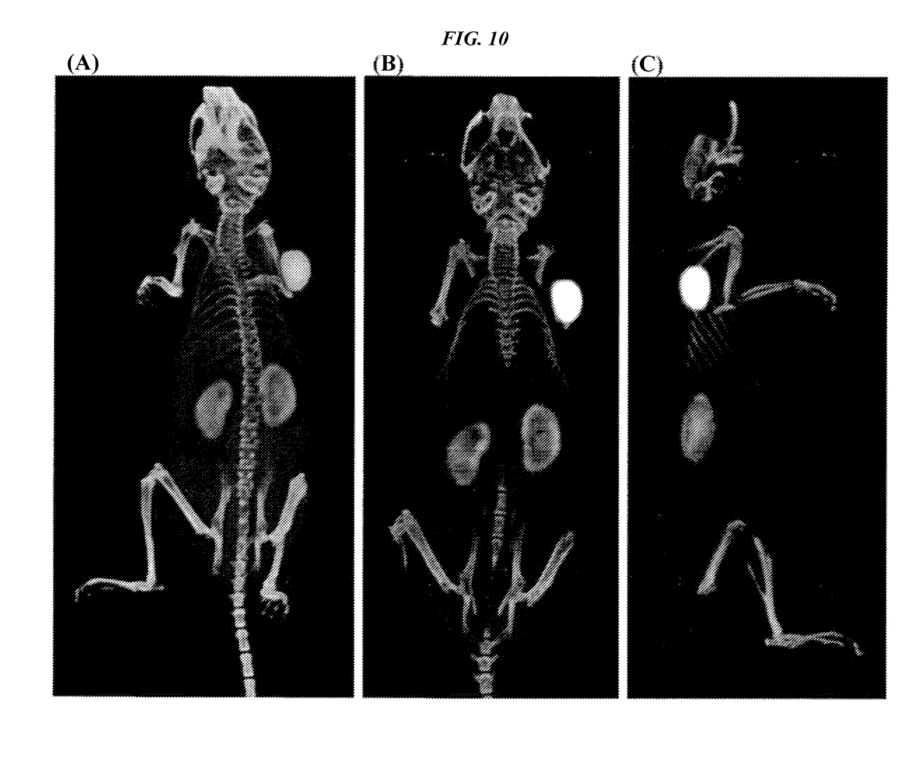

The PET-images of pretargeted immunoPET with Al¹⁸F(IMP449) show the same intensity in the tumor as those with ⁶⁸Ga(IMP288), but the resolution of the ¹⁸F PET images were superior to those of the ⁶⁸Ga. (FIG. 10). The tumor-to-background ratio of the Al¹⁸F(IMP449) signal was 66.

### CONCLUSIONS

The present study showed that pretargeted immunoPET with the anti-CEA x anti-HSG bispecific antibody TF2 in combination with a ⁶⁸Ga- or ¹⁸F-labeled di-HSG-DOTA-peptide is a rapid and specific technique for PET imaging of CEA-expressing tumors.

Pretargeted immunoPET with TF2 in combination with ⁶⁸Ga(IMP288) or Al¹⁸F(IMP449) involves two intravenous administrations. An interval between the infusion of the bsMAb and the radiolabeled peptide of 16 h was used. After 16 h most of the TF2 had cleared from the blood (blood concentration<1% ID/g), preventing complexation of TF2 and IMP288 in the circulation.

For these studies the procedure to label IMP288 with ⁶⁸Ga was optimized, resulting in a one-step labeling technique. We found that purification on a C18/HLB cartridge was needed to remove the ⁶⁸Ga colloid that is formed when the peptide was labeled at specific activities exceeding 150 GBq/nmol at 95° C. If a preparation contains a small percentage of colloid and is administered intravenously, the ⁶⁸Ga colloid accumulates in tissues of the mononuclear phagocyte system (liver, spleen, and bone marrow), deteriorating image quality. The ⁶⁸Ga-labeled peptide could be rapidly purified on a C18-cartridge. Radiolabeling and purification for administration could be accomplished within 45 minutes.

The half-life of ⁶⁸Ga matches with the kinetics of the IMP288 peptide in the pretargeting system: maximum accretion in the tumor is reached within 1 h. ⁶⁸Ga can be eluted twice a day form a ⁶⁸Ge/⁶⁸Ga generator, avoiding the need for an on-site cyclotron. However, the high energy of the positrons emitted by ⁶⁸Ga (1.9 MeV) limits the spatial resolution of the acquired images to 3 mm, while the intrinsic resolution of the microPET system is as low as 1.5 mm.

¹⁸F, the most widely used radionuclide in PET, has an even more favorable half-life for pretargeted PET imaging (t_{1/2}=110 min). The NOTA-conjugated peptide IMP449 was labeled with ¹⁸F, as described above. Like labeling with ⁶⁸Ga, it is a one-step procedure. Labeling yields as high as 50% were obtained. The biodistribution of Al¹⁸F(IMP449) was highly similar to that of ⁶⁸Ga-labeled IMP288, suggesting that with this labeling method ¹⁸F is a residualizing radionuclide.

In contrast with FDG-PET, pretargeted radioimmunodetection is a tumor specific imaging modality. Although a high sensitivity and specificity for FDG-PET in detecting recurrent colorectal cancer lesions has been reported in patients (Huebner et al., 2000, J Nucl Med 41:11277-89), FDG-PET images could lead to diagnostic dilemmas in discriminating malignant from benign lesions, as indicated by the high level of labeling observed with inflammation. In contrast, the high tumor-to-background ratio and clear visualization of CEA-positive tumors using pretargeted immunoPET with TF2 ⁶⁸Ga- or ¹⁸F-labeled peptides supports the use of the described methods for clinical imaging of cancer and other conditions. Apart from detecting metastases and discriminating CEA-positive tumors from other lesions, pretargeted immunoPET could also be used to estimate radiation dose delivery to tumor and normal tissues prior to pretargeted radioimmunotherapy. As TF2 is a humanized antibody, it has a low immunogenicity, opening the way for multiple imaging or treatment cycles.

### Example 18

### Synthesis of Folic Acid NOTA Conjugate

Folic acid is activated as described (Wang et. al. Bioconjugate Chem. 1996, 7, 56-62.) and conjugated to Boc-NH—CH₂—CH₂—NH₂. The conjugate is purified by chromatography. The Boc group is then removed by treatment with TFA. The amino folate derivative is then mixed with p-SCN-Bn-NOTA (Macrocyclics) in a carbonate buffer. The product is then purified by HPLC. The folate-NOTA derivative is labeled with Al¹⁸F as described in the preceding Examples and then HPLC purified. The ¹⁸F-labeled folate is injected i.v. into a subject and successfully used to image the distribution of folate receptors, for example in cancer or inflammatory diseases (see, e.g., Ke et al., Advanced Drug Delivery Reviews, 56:1143-60, 2004).

### Example 19

### Imaging of Angiogenesis Receptors by ¹⁸ F-Labeling

Labeled Arg-Gly-Asp (RGD) peptides have been used for imaging of angiogenesis, for example in ischemic tissues, where αᵥβ₃ integrin is involved. (Jeong et al., J. Nucl. Med. 2008, Apr. 15 epub). RGD is conjugated to SCN-Bn-NOTA according to Jeong et al. (2008). [Al¹⁸F] is attached to the NOTA-derivatized RGD peptide as described above, by mixing aluminum stock solution with ¹⁸F and the derivatized RGD peptide and heating at 110° C. for 15 min, using an excess of peptide to drive the labeling reaction towards completion. The ¹⁸F labeled RGD peptide is used for in vivo biodistribution and PET imaging as disclosed in Jeong et al. (2008). The [Al¹⁸F] conjugate of RGD-NOTA is taken up into ischemic tissues and provides PET imaging of angiogenesis.

### Example 20

### Effect of Organic Solvents on F-18 Labeling

The affinity of chelating moieties such as NETA and NOTA for aluminum is much higher than the affinity of aluminum for ¹⁸F. The affinity of Al for ¹⁸F is affected by factors such as the ionic strength of the solution, since the presence of other counter-ions tends to shield the positively charged aluminum and negatively charged fluoride ions from each other and therefore to decrease the strength of ionic binding. Therefore low ionic strength medium should increase the effective binding of Al and ¹⁸F.

An initial study adding ethanol to the ¹⁸F reaction was found to increase the yield of radiolabeled peptide. IMP461 was prepared as described above.

TABLE 4
¹⁸F-labeling of IMP461 in ethanol

| | 2 mM | | 2 mM | | | |
|---|---|---|---|---|---|---|
| # | AlCl₃ | ¹⁸F⁻ | IMP 461 | Solvent | | Yield* |
| 1 | 10 μL | 741 μCi | 20 μL | EtOH | 60 μL | 64.9% |
| 2 | 10 μL | 739 μCi | 20 μL | H₂O | 60 μL | 21.4% |
| 3 | 10 μL | 747 μCi | 20 μL | EtOH | 60 μL | 46.7% |
| 4 | 5 μL | 947 μCi | 10 μL | EtOH | 60 μL | 43.2% |

*Yield after HLB column purification, Rxn # 1, 2 and 4 were heated to 101° C. for 5 minutes, Rxn # 3 was heated for 1 minute in a microwave oven.

Preliminary results showed that addition of ethanol to the reaction mixture more than doubled the yield of ¹⁸F-labeled peptide. Table 4 also shows that microwave irradiation can be used in place of heating to promote incorporation of [Al¹⁸F] into the chelating moiety of IMP461. Sixty seconds of microwave radiation (#3) appeared to be slightly less (18%) effective than heating to 101° C. for 5 minutes (#1).

The effect of additional solvents on Al¹⁹F complexation of peptides was examined. In each case, the concentration of reactants was the same and only the solvent varied. Reaction conditions included mixing 25 μL Na¹⁹F+20 μL AlCl₃+20 μL IMP461+60 μL solvent, followed by heating at 101° C. for 5 min. Table 5 shows that the presence of a solvent does improve the yields of Al¹⁹F(IMP461) (i.e., IMP473) considerably.

TABLE 5
Complexation of IMP 461 with Al¹⁹F in various solvents

| Solvent | H₂O | MeOH | EtOH | CH₃CN |
|---|---|---|---|---|
| Al-IMP461 | 2.97 | 3.03 | 2.13 | 1.54 |
| IMP465 | 52.46 | 34.19 | 31.58 | 24.58 |
| IMP473 | 14.99 | 30.96 | 33.00 | 37.48 |
| IMP473 | 15.96 | 31.81 | 33.29 | 36.40 |
| IMP461 | 13.63 | — | — | — |
| Solvent | IPA | Acetone | THF | Dioxane |
| Al-IMP461 | 2.02 | 2.05 | 2.20 | 16.67 |
| IMP465 | 32.11 | 28.47 | 34.76 | 10.35 |
| IMP473 | 27.31 | 34.35 | 29.38 | 27.09 |
| IMP473 | 27.97 | 35.13 | 29.28 | 11.62 |
| IMP461 | 10.58 | — | 4.37 | 34.27 |

| Solvent | DMF | DMSO | t_{R} (min) |
|---|---|---|---|
| Al-IMP461 | — | — | 9.739 |
| IMP465 | 19.97 | 37.03 | 10.138 |
| IMP473 | 27.77 | 31.67 | 11.729 |
| IMP473 | 27.34 | 31.29 | 11.952 |
| IMP461 | — | — | 12.535 |

Al[¹⁹F]IMP461 = IMP473

### Example 21

### Elution of ¹⁸F⁻ with Bicarbonate

¹⁸F, 10.43 mCi, was received in 2 mL in a syringe. The solution was passed through a SEP-PAK® Light, WATERS® ACCELL™ Plus QMA Cartridge. The column was then washed with 5 mL of DI water. The ¹⁸F was eluted with 0.4 M KHCO₃ in fractions as shown in Table 6 below.

TABLE 6
Elution of QMA Cartridge with KHCO₃

| | Vol. Acetic | Vol. 0.4M | |
|---|---|---|---|
| Vial | acid μL | KHCO₃ μL | Activity mCi |
| 1 | 7.5 | 150 | 0.0208 |
| 2 | 10 | 200 | 7.06 |
| 3 | 5 | 100 | 1.653 |
| 4 | 25 | 500 | 0.548 |

The effects of the amount of additional solvent (CH₃CN) on ¹⁸F-labeling of IMP461 was examined. In each case, the concentration of reactants was the same and only the amount of solvent varied. Reaction conditions included mixing 10 μL AlCl₃+20 μL ¹⁸F+20 μL IMP461+CH₃CN followed by heating at 101° C. for 5 min. Table 7 shows that following an initial improvement the labeling efficiency decreases in the presence of excess solvent.

TABLE 7
¹⁸F-labeling of IMP461 using varying amounts of CH₃CN

| CH₃CN | | t_{R} 2.70 min | t_{R} 8.70 min | RCY % |
|---|---|---|---|---|
| (μL) | ¹⁸F⁻ mCi | (%) | (%) | (HLB) |
| 0 | 0.642 | 13.48 | 86.52 | 50.7 |
| 100 | 0.645 | 1.55 | 98.45 | 81.8* |
| 200 | 0.642 | 2.85 | 97.15 | 80.8 |
| 400 | 0.645 | 14.51 | 85.49 | 57.8 |

*Aqueous wash contains labeled peptide. RCY = radiochemical yield after HLB purification

### Example 22

### High Dose Radiolabeling of IMP461

18F⁻, 163 mCi, was received in 2 mL in a syringe. The solution was passed through a SEP-PAK® Light, WATERS® ACCELL™ Plus QMA Cartridge. The column was then washed with 5 mL of DI water. The ¹⁸F⁻ was eluted with 0.4 M K₂CO₃ in fractions as shown in Table 8.

TABLE 8
High Dose Labeling

| | Vol. Acetic | Vol. 0.4M | |
|---|---|---|---|
| Vial | acid μL | K₂CO₃ μL | Activity mCi |
| 1 | 18.5 | 185 | 5.59 |
| 2 | 5 | 50 | 35.8 |
| 3 | 5 | 50 | 59.9 |
| 4 | 5 | 50 | 20.5 |
| 5 | 5 | 50 | 5.58 |
| 6 | 50 | 500 | 4.21 |

An aluminum chloride solution (10 μL, 2 mM in pH 4, 2 mM NaOAc) was added to vial number 3 from Table 8. The peptide (20 μL, 2 mM in pH 4, 2 mM NaOAc) was added to the vial followed by the addition of 170 μL of CH₃CN. The solution was heated for 10 min at 103° C. the diluted with 6 mL of water. The solution was pulled into a 10 mL syringe and injected onto two WATERS® HLB Plus Cartridges arranged in tandem. The cartridges were washed with 8 mL water. The radiolabeled peptide Al¹⁸F(IMP461) was then eluted with 10 mL 1:1 EtOH/H₂O, 30.3 mCi, 63.5% yield, specific activity 750 Ci/mmol. The labeled peptide was free of unbound ¹⁸F by HPLC. The total reaction and purification time was 20 min.

### Example 23

### Preparation of Al¹⁹F Peptides

Products containing ²⁷Al and/or ¹⁹F are useful for certain applications like MR imaging. An improved method for preparing [Al¹⁹F] compounds was developed. IMP461 was prepared as described above and labeled with ¹⁹F. Reacting IMP461 with AlCl₃+NaF resulted in the formation of three products (not shown). However, by reacting IMP461 with AlF₃⁻3H₂O we obtained a higher yield of Al¹⁹F(IMP461).

Synthesis of IMP 473: [Al¹⁹F(IMP461)] To (14.1 mg, 10.90 μmol) IMP461 in 2 mL NaOAc (2 mM, pH 4.18) solution added (4.51 mg, 32.68 μmol) AlF₃3H₂O and 500 μL ethanol. The pH of the solution to adjusted to 4.46 using 3μL 1 N NaOH and heated in a boiling water bath for 30 minutes. The crude reaction mixture was purified by preparative RP-HPLC to yield 4.8 mg (32.9%) of IMP 473. HRMS (ESI-TOF) MH⁺ expected 1337.6341; found 1337.6332

These results demonstrate that ¹⁹F labeled molecules may be prepared by forming metal-¹⁹F complexes and binding the metal-¹⁹F to a chelating moiety, as discussed above for ¹⁸F labeling. The instant Example shows that a targeting peptide of use for pretargeting detection, diagnosis and/or imaging may be prepared using the instant methods.

### Example 24

### Synthesis and Labeling of IMP479, IMP485 and IMP487

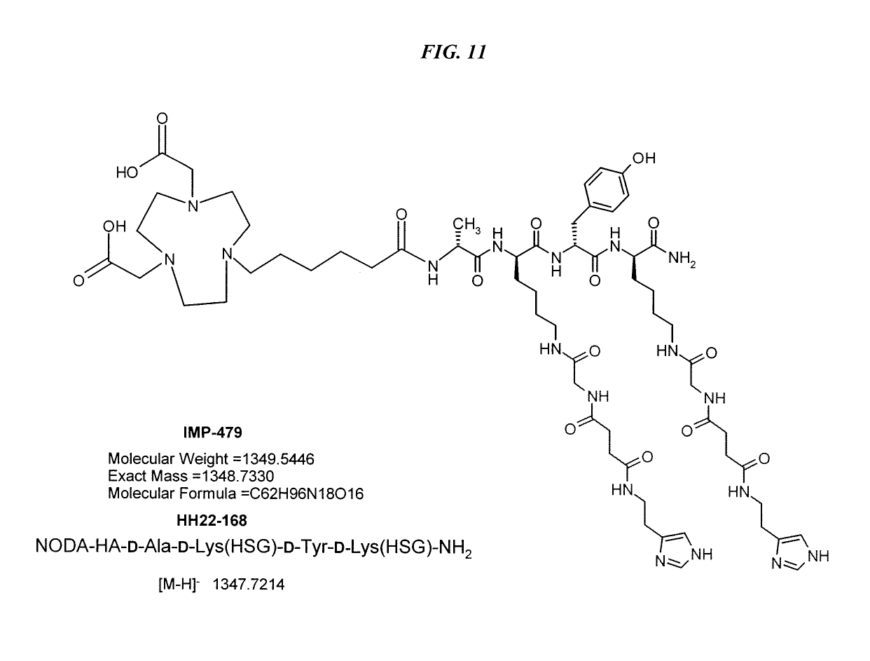

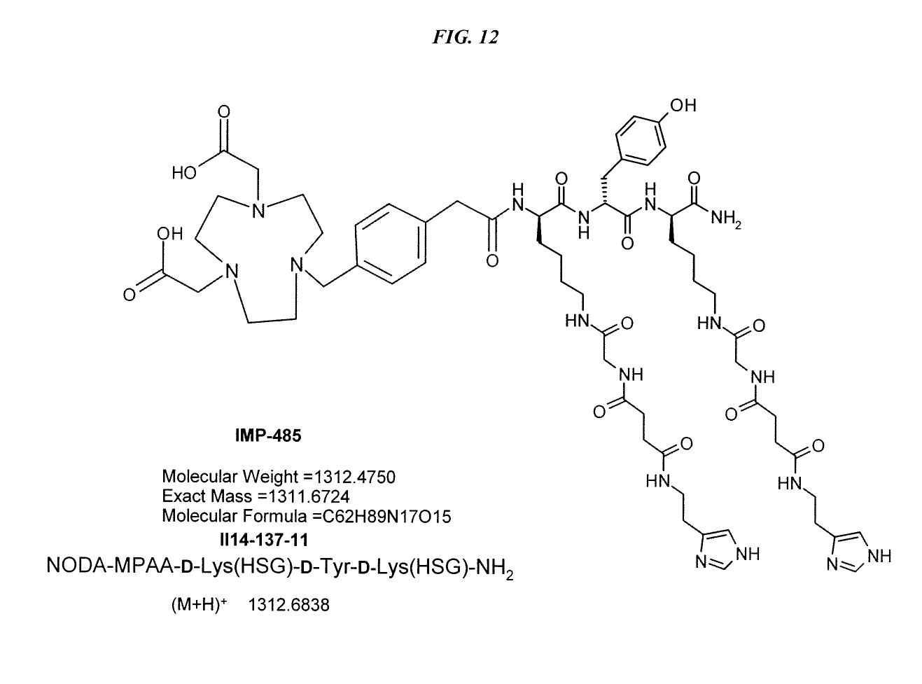

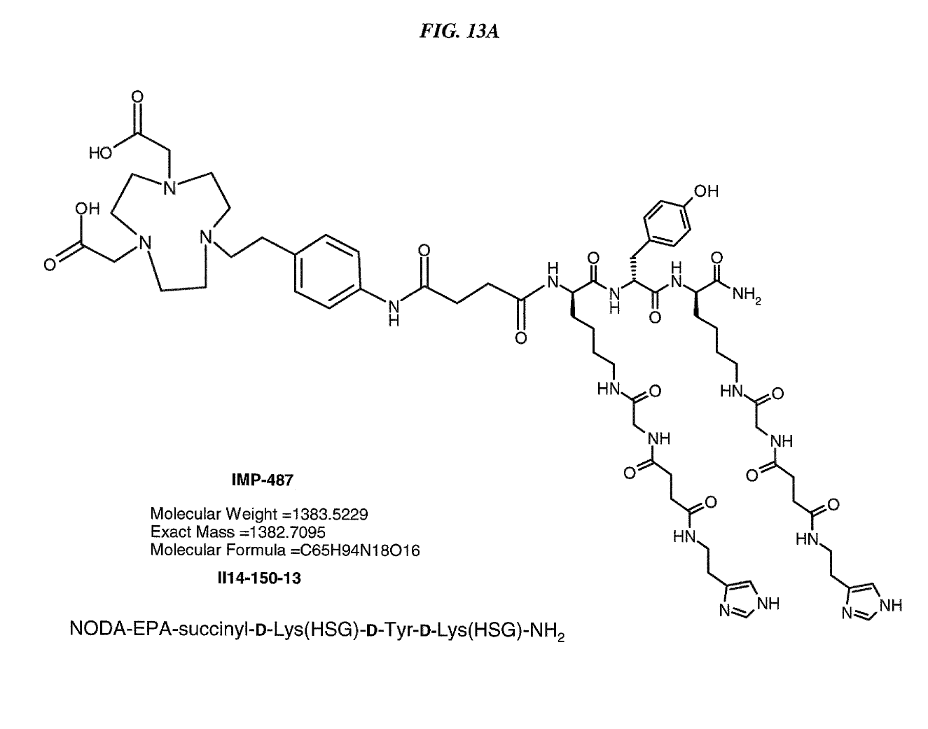

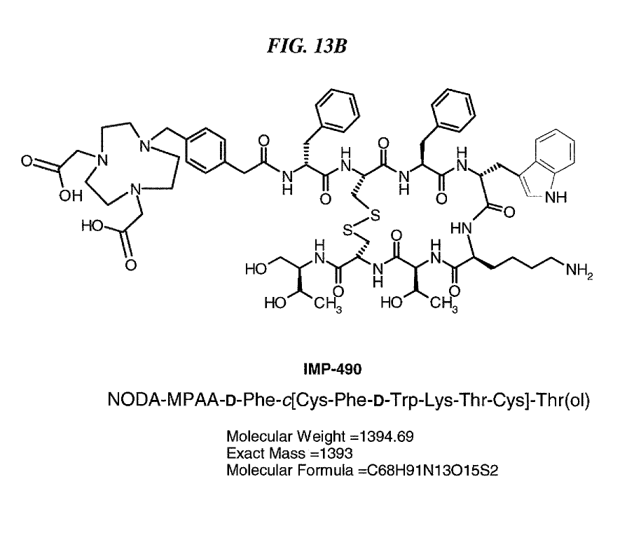

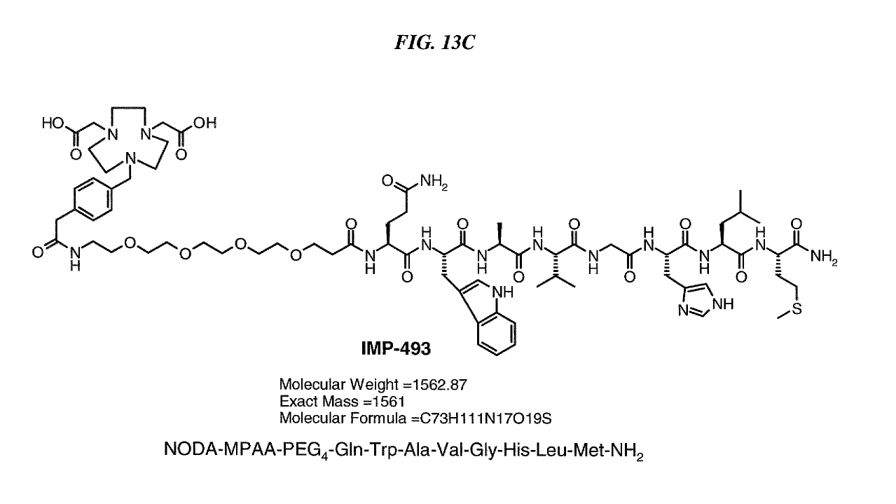

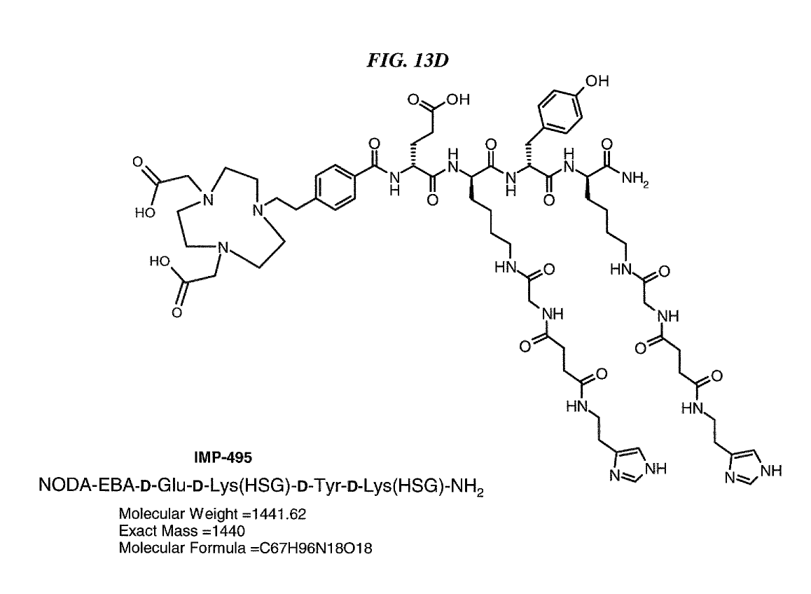

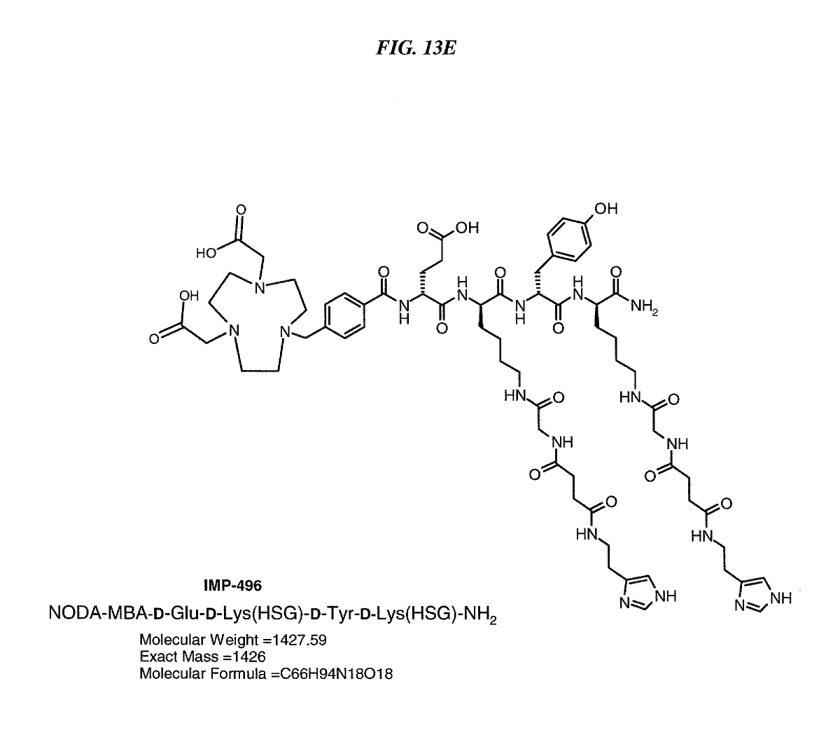

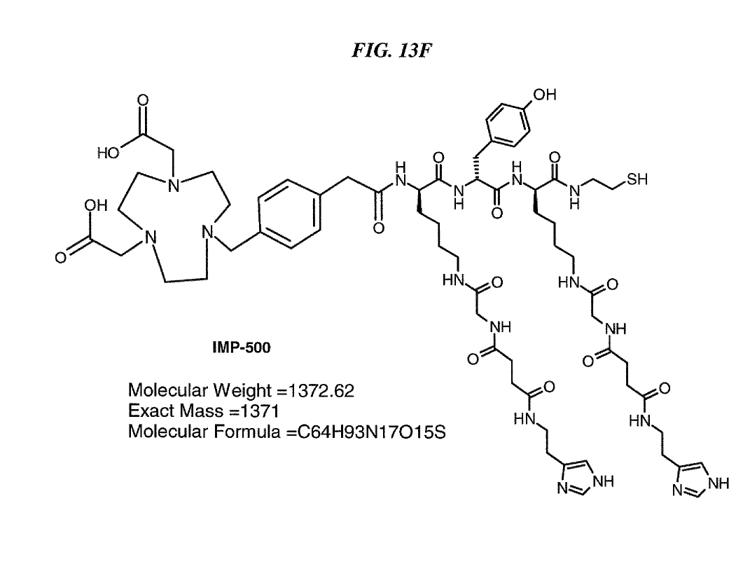

The structures of additional peptides (IMP479, IMP485, and IMP487) designed for ¹⁸F-labeling are shown in FIG. 11 to FIG. 13. IMP485 is shown in FIG. 12. IMP485 was made on Sieber Amide resin by adding the following amino acids to the resin in the order shown: Aloc-D-Lys(Fmoc)-OH, Trt-HSG-OH, the Aloc was cleaved, Fmoc-D-Tyr(But)-OH, Aloc-D-Lys(Fmoc)-OH, Trt-HSG-OH, the Aloc was cleaved, (tert-Butyl)₂NOTA-MPAA (methyl phenyl acetic acid). The peptide was then cleaved from the resin and purified by RP-HPLC to yield 44.8 mg of IMP485.

Synthesis of Bis-t-butyl-NOTA-MPAA: (tBu)₂NOTA-MPAA for IMP485 Synthesis

To a solution of 4-(bromomethyl) phenyl acetic acid (Aldrich 310417) (0.5925 g, 2.59 mmol) in CH₃CN (anhydrous) (50 mL) at 0° C. was added dropwise over 1 h a solution of NO2AtBu (1.0087 g, 2.82 mmol) in CH₃CN (50 mL). After 4 h anhydrous K₂CO₃ (0.1008 g, 0.729 mmol) was added to the reaction mixture and allowed to stir at room temperature overnight. Solvent was evaporated and the crude mixture was purified by preparative RP-HPLC to yield a white solid (0.7132 g, 54.5%).

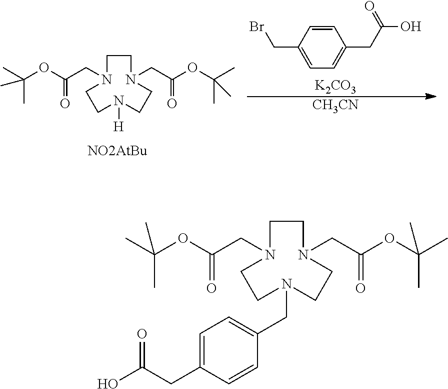

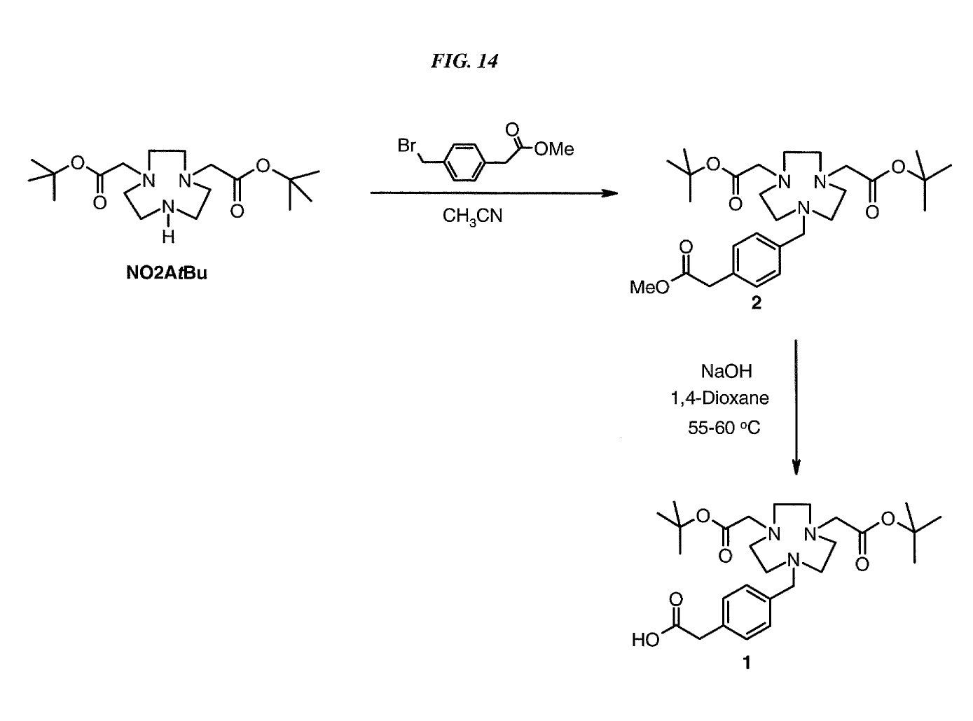

Although this is a one step synthesis, yields were low due to esterification of the product by 4-(bromomethyl)phenylacetic acid. Alkylation of NO2AtBu using methyl(4-bromomethyl)phenylacetate was employed to prevent esterification (FIG. 14).

### ¹⁸F-Labeling

For ¹⁸F labeling studies in water, to 40 nmol of IMP479/485/487 (formulated using trehalose+ascorbic acid+AlCl₃) was added 250 μL ¹⁸F⁻ solution [˜919-1112 μCi of ¹⁸F⁻] and heated to 101° C. for 15 minutes. In ethanol, to 40 nmol of IMP479/485/487 (formulated using trehalose+ascorbic acid+AlCl₃) was added 250 μL ¹⁸F⁻ solution [1.248-1.693 mCi of ¹⁸F⁻], 100 μL EtOH and heated to 101° C. for 15 minutes. An exemplary experiment showing labeling of different peptides is shown in Table 9. With minimal optimization, radiolabeling of IMP485 has been observed with up to an 88% yield and a specific activity of 2,500 Ci/mmol. At this specific activity, HPLC purification of the radiolabeled peptide is not required for in vivo PET imaging using the radiolabeled peptide.

TABLE 9
Labeling of IMP479, IMP485 and IMP487

Isolated yields after
HLB purification

| IMP # | H₂O | EtOH |
|---|---|---|
| IMP479 | 44.0% | 57.5% |
| IMP485 | 74.4% | 79.7% |
| IMP487 | 63.6% | 81.6% |

### Stability in Serum

A kit containing 40 nmol of IMP485 or IMP487, 20 nmol AlCl₃, 0.1 mg ascorbic acid and 0.1 g trehalose adjusted to pH 3.9 was reconstituted with purified ¹⁸F⁻ in 200 μL saline and heated 106° C. for 15 min. The reaction mixture was then diluted with 800 μL water and placed on an HLB column. After washing, the column was eluted with 2×200 μL 1:1 EtOH/H₂O to obtain the purified Al¹⁸F(IMP485) in 64.6% isolated yield. The radiolabeled peptide in 50 μL was mixed with 250 μL of fresh human serum in a vial and incubated at 37° C.

Both radiolabeled peptides were stable at 37° C. in fresh human serum over the four hours tested (not shown).

### Effect of Bulking Agents on Yield of Lyophilized Peptide

An experiment was performed to compare yield using IMP485 kits (40 nmol) with different bulking agents labeled with 2 mCi of F-18 (from the same batch of F-18) in 200 microliters of saline. The bulking agents were introduced at a concentration of 5% by weight in water with a dose of 200 microliters/vial. We tested sorbitol, trehalose, sucrose, mannitol and glycine as bulking agents. Results are shown in Table 10

TABLE 10
Effects of Bulking Agents on Radiolabeling Yield

| Bulking Agent | Activity mCi | Yield % |
|---|---|---|
| Sorbitol | 2.17 | 82.9 |
| Glycine | 2.17 | 41.5 |
| Mannitol | 2.11 | 81.8 |
| Sucrose | 2.11 | 66.1 |
| Trehalose | 2.10 | 81.3 |

Sorbitol, mannitol and trehalose all gave radiolabeled product in the same yield. The mannitol kit and the trehalose kit both formed nice cakes. The sucrose kit and the glycine kit both had significantly lower yields. We also recently tested 2-hydroxypropyl-beta-cyclodextrin as a bulking agent and obtained a 58% yield for the 40 nmol kit. We have found that radiolabeling is very pH sensitive and needs to be tuned to the ligand and possibly even to the peptide +the ligand. In the case of IMP485 the optimal pH is pH 4.0±0.2 whereas the optimal pH for IMP467 was pH 4.5±0.5. In both cases the yields drop off rapidly outside the ideal pH zone.

### Time Course of Labeling

The time course for labeling of IMP485 was examined. To 40 nmol of IMP485 (formulated using trehalose+AlCl₃ (20 nmol) +ascorbic acid) was added ˜200-250 μL ¹⁸F⁻ solution (0.9% saline) and heated to 104° C. for 5 to 15 minutes. The results for labeling yield were: 5 min (28.9%), 10 min (57.9%), 15 min (83.7%) and 30 min (88.9%). Thus, the time course for labeling was approximately 15 minutes.

### Biodistribution of IMP485 Alone

The biodistribution of IMP485 in the absence of any pretargeting antibody was examined in female Taconic nude mice (10 week old) bearing small or no BXPC3 pancreatic cancer xenografts. The mice were injected i.v. with Al¹⁸F(IMP485), (340 μCi, 2.29×10⁻⁹ mol, 100 μL in saline). The mice, 6 per time point, were necropsied at 30 min and 90 min post injection. In the absence of pretargeting antibody a low level of accumulation was seen in tumor and most normal tissues. The substantial majority of radiolabel was found in the bladder and to a lesser extent in kidney. Most of the activity was cleared before the 90 min time point.

Pretargeting of IMP485 with TF2 DNL Targeting Molecule

IMP485 Radiolabeling—¹⁸F⁻ (218 mCi) was purified to isolate 145.9 mCi. The purified ¹⁸F⁻ (135 mCi) was added to a lyophilized vial containing 40 nmol of pre-complexed Al(IMP485). The reaction vial was heated at 110° C. for 17 min. Water (0.8 mL) was added to the reaction mixture before HLB purification. The product (22 mCi) was eluted with 0.6 mL of water:ethanol (1:1) mixture into a vial containing lyophilized ascorbic acid. The product was diluted with saline. The Al¹⁸F(IMP485)specific activity used for injection was 550 Ci/mmol.

Biodistribution of Al¹⁸F(IMP485) alone—Mice bearing sc LS174T xenografts were injected with Al¹⁸F(IMP485) (28 μCi, 5.2×10⁻¹¹ mol, 100 μL. Mice were necropsied at 1 and 3 h post injection, 6 mice per time point.

Biodistribution of TF2+Al¹⁸F(IMP485) With Pretargeting at 20:1 bsMAb to peptide ratio - Mice bearing sc LS174T xenografts were injected with TF2 (163.2 μg, 1.03×10⁻⁹ mol, iv) and allowed 16.3 h for clearance before injecting Al¹⁸F(IMP485) (28 Ci, 5.2×10⁻¹¹ mol, 100 μL iv). Mice were necropsied at 1 and 3 h post injection, 7 mice per time point.

Urine stability—Ten mice bearing s.c. Capan-1 xenografts were injected with Al¹⁸F(IMP485) (400 Ci, in saline, 100 μL). Urine was collected from 3 mice at 55 min post injection. The urine samples were analyzed by reverse phase and SE-HPLC. Stability of the radiolabeled IMP485 in urine was observed.

TABLE 11
Al¹⁸F(IMP485) Alone at 1 h post injection:

| | | | | | STD | | STD | | |
|---|---|---|---|---|---|---|---|---|---|
| | | | | % | % | % | % | | STD |
| | | | STD | ID/ | ID/ | ID/ | ID/ | T/ | T/ |
| Tissue | n | Weight | WT | g | g | org | org | NT | NT |
| Tumor | 6 | 0.235 | 0.147 | 0.316 | 0.114 | 0.081 | 0.063 | 1.0 | 0.0 |
| Liver | 6 | 1.251 | 0.139 | 0.176 | 0.032 | 0.220 | 0.043 | 1.8 | 0.4 |
| Spleen | 6 | 0.085 | 0.019 | 0.210 | 0.181 | 0.018 | 0.017 | 1.9 | 0.9 |
| Kidney | 6 | 0.149 | 0.013 | 3.328 | 0.556 | 0.499 | 0.119 | 0.1 | 0.0 |
| Lung | 6 | 0.141 | 0.039 | 0.238 | 0.048 | 0.033 | 0.010 | 1.3 | 0.3 |
| Blood | 6 | 0.222 | 0.006 | 0.165 | 0.062 | 0.268 | 0.101 | 2.0 | 0.4 |
| Stomach | 6 | 0.478 | 0.083 | 0.126 | 0.110 | 0.057 | 0.045 | 3.5 | 1.6 |
| Sm Int. | 6 | 0.896 | 0.098 | 0.396 | 0.128 | 0.353 | 0.110 | 0.8 | 0.3 |
| Lg Int. | 6 | 0.504 | 0.056 | 0.081 | 0.019 | 0.041 | 0.010 | 3.9 | 0.9 |
| Muscle | 6 | 0.103 | 0.029 | 0.114 | 0.079 | 0.011 | 0.008 | 4.1 | 2.5 |
| Scapula | 6 | 0.057 | 0.015 | 0.107 | 0.019 | 0.006 | 0.001 | 2.9 | 0.7 |

TABLE 12
Al¹⁸F(IMP485) Alone at 3 h post injection:

| | | | | | STD | | STD | | |
|---|---|---|---|---|---|---|---|---|---|
| | | | | % | % | % | % | | STD |
| | | | STD | ID/ | ID/ | ID/ | ID/ | T/ | T/ |
| Tissue | n | Weight | WT | g | g | org | org | NT | NT |
| Tumor | 6 | 0.265 | 0.126 | 0.088 | 0.020 | 0.022 | 0.011 | 1.0 | 0.0 |
| Liver | 6 | 1.219 | 0.091 | 0.095 | 0.047 | 0.114 | 0.056 | 13.6 | 31.4 |
| Spleen | 6 | 0.091 | 0.015 | 0.065 | 0.009 | 0.006 | 0.001 | 1.4 | 0.2 |
| Kidney | 6 | 0.154 | 0.013 | 2.265 | 0.287 | 0.345 | 0.028 | 0.0 | 0.0 |
| Lung | 6 | 0.142 | 0.008 | 0.073 | 0.019 | 0.010 | 0.003 | 1.3 | 0.6 |
| Blood | 6 | 0.236 | 0.019 | 0.008 | 0.005 | 0.013 | 0.007 | 21.0 | 27.9 |
| Stomach | 6 | 0.379 | 0.054 | 0.041 | 0.017 | 0.016 | 0.008 | 2.5 | 1.0 |
| Sm. Int. | 6 | 0.870 | 0.042 | 0.137 | 0.031 | 0.119 | 0.029 | 0.7 | 0.3 |
| Lg. Int. | 6 | 0.557 | 0.101 | 0.713 | 0.215 | 0.408 | 0.194 | 0.1 | 0.0 |
| Muscle | 6 | 0.134 | 0.038 | 0.013 | 0.007 | 0.002 | 0.001 | 203.9 | 486.6 |
| Scapula | 6 | 0.074 | 0.009 | 0.079 | 0.026 | 0.006 | 0.002 | 1.2 | 0.6 |

TABLE 13
TF2 + Al¹⁸F(IMP485), at 1 h post peptide injection:

| | | | | | STD | | STD | | |
|---|---|---|---|---|---|---|---|---|---|
| | | | | % | % | % | % | | STD |
| | | | STD | ID/ | ID/ | ID/ | ID/ | T/ | T/ |
| Tissue | n | Weight | WT | g | g | org | org | NT | NT |
| Tumor | 7 | 0.291 | 0.134 | 28.089 | 4.545 | 8.025 | 3.357 | 1 | 0 |
| Liver | 7 | 1.261 | 0.169 | 0.237 | 0.037 | 0.295 | 0.033 | 123 | 38 |
| Spleen | 7 | 0.081 | 0.013 | 0.254 | 0.108 | 0.020 | 0.008 | 139 | 87 |
| Kidney | 7 | 0.140 | 0.018 | 3.193 | 0.730 | 0.444 | 0.098 | 9 | 4 |
| Lung | 7 | 0.143 | 0.014 | 0.535 | 0.147 | 0.075 | 0.018 | 57 | 22 |
| Blood | 7 | 0.205 | 0.029 | 0.278 | 0.071 | 0.456 | 0.129 | 110 | 43 |
| Stomach | 7 | 0.473 | 0.106 | 0.534 | 1.175 | 0.265 | 0.598 | 381 | 318 |
| Sm. Int. | 7 | 0.877 | 0.094 | 0.686 | 0.876 | 0.586 | 0.725 | 75 | 39 |
| Lg. Int. | 7 | 0.531 | 0.068 | 0.104 | 0.028 | 0.055 | 0.015 | 291 | 121 |
| Muscle | 7 | 0.090 | 0.014 | 0.136 | 0.102 | 0.012 | 0.009 | 348 | 274 |
| Scapula | 6 | 0.189 | 0.029 | 0.500 | 0.445 | 0.095 | 0.092 | 120 | 108 |

TABLE 14
TF2 + Al¹⁸F(IMP485), at 3 h post peptide injection:

| | | | | | STD | | | | |
|---|---|---|---|---|---|---|---|---|---|
| | | | | % | % | % | STD | | STD |
| | | | STD | ID/ | ID/ | ID/ | ID/ | T/ | T/ |
| Tissue | n | Weight | WT | g | g | org | org | NT | NT |
| Tumor | 7 | 0.320 | 0.249 | 26.518 | 5.971 | 8.127 | 5.181 | 1 | 0 |
| Liver | 7 | 1.261 | 0.048 | 0.142 | 0.019 | 0.178 | 0.025 | 189 | 43 |
| Spleen | 7 | 0.079 | 0.012 | 0.138 | 0.031 | 0.011 | 0.002 | 195 | 41 |
| Kidney | 7 | 0.144 | 0.012 | 2.223 | 0.221 | 0.319 | 0.043 | 12 | 3 |
| Lung | 7 | 0.145 | 0.014 | 0.244 | 0.056 | 0.035 | 0.005 | 111 | 24 |
| Blood | 7 | 0.229 | 0.014 | 0.023 | 0.008 | 0.037 | 0.012 | 1240 | 490 |
| Stomach | 7 | 0.430 | 0.069 | 0.025 | 0.017 | 0.010 | 0.005 | 1389 | 850 |
| Sm. Int. | 7 | 0.818 | 0.094 | 0.071 | 0.029 | 0.059 | 0.028 | 438 | 207 |
| Lg. Int. | 7 | 0.586 | 0.101 | 0.353 | 0.160 | 0.206 | 0.103 | 86 | 33 |
| Muscle | 7 | 0.094 | 0.014 | 0.025 | 0.006 | 0.002 | 0.001 | 1129 | 451 |
| Scapula | 7 | 0.140 | 0.030 | 0.058 | 0.018 | 0.008 | 0.002 | 502 | 193 |

### Conclusions

The IMP485 labels as well as or better than IMP467, with equivalent stability in serum. However, IMP485 is much easier to synthesize than IMP467. Preliminary studies have shown that ¹⁸F-labeling of lyophilized IMP485 works as well as non-lyophilized peptide (data not shown). The presence of alkyl or aryl groups in the linker joining the chelating moiety to the rest of the peptide was examined. The presence of aryl groups in the linker appears to increase the radiolabeling yield relative to the presence of alkyl groups in the linker.

Biodistribution of IMP485 in the presence or absence of pretargeting antibody resembles that observed with IMP467. In the absence of pretargeting antibody, distribution of radiolabeled peptide in tumor and most normal tissues is low and the peptide is removed from circulation by kidney excretion. In the presence of the TF2 antibody, radiolabeled IMP485 is found primarily in the tumor, with little distribution to normal tissues. Kidney radiolabeling is substantially decreased in the presence of the pretargeting antibody. We conclude that IMP485 and other peptides with aryl groups in the linker are highly suitable for PET imaging with ¹⁸F-labeling.

### Example 25

### Kit Formulation of IMP485 for Imaging

We report a simple, general kit formulation for labeling peptides with ¹⁸F. A ligand that contains 1,4,7-triazacyclononane-N,N′, N″-triacetic acid (NOTA) attached to a methyl phenylacetic acid (MPAA) group was used to form a single stable complex with (AlF)²⁺. The lyophilized kit contained IMP485, a di-HSG hapten-peptide used for pretargeting. The kit was reconstituted with an aqueous solution of ¹⁸F⁻, heated at 100-110° C. for 15 min, followed by a rapid purification by solid-phase extraction (SPE). In vitro and in vivo stability and tumor targeting of the Al¹⁸F(IMP485) were examined in nude mice bearing human colon cancer xenografts pretargeted with an anti-CEACAM5 bispecific antibody. ¹⁸F-labeling of MPAA-bombesin and somatostatin peptides also was evaluated.

The HSG peptide was labeled with ¹⁸F⁻ as a single isomer complex, in high yield (50-90%) and high specific activity (up to 153 GBq/μmol), within 30 min. It was stable in human serum at 37° C. for 4 h, and in vivo showed low uptake (0.06%±0.02 ID/g) in bone. At 3 h, pretargeted animals had high Al¹⁸F(IMP485) tumor uptake (26.5%±6.0 ID/g), with ratios of 12±3, 189±43, 1240±490 and 502±193 for kidney, liver, blood and bone, respectively. Bombesin and octreotide analogs were labeled with comparable yields. In conclusion, ¹⁸F-labeled peptides can be produced as a stable, single [Al¹⁸F] complex with good radiochemical yields and high specific activity in a simple one-step kit.

### Reagents List

Reagents were obtained from the following sources: Acetic acid (J T Baker 6903-05 or 9522-02), Sodium hydroxide (Aldrich semiconductor grade 99.99% 30,657-6), α,α-Trehalose (J T Baker 4226-04), Aluminum chloride hexahydrate (Aldrich 99% 237078), Ascorbic acid (Aldrich 25,556-4).

Acetate Buffer 2 mM—Acetic acid, 22.9 μL (4.0×10⁻⁴ mol) was diluted with 200 mL water and neutralized with 6 N NaOH (˜15 μL) to adjust the solution to pH 4.22.

Aluminum Solution 2 mM—Aluminum hexahydrate, 0.0225 g (9.32×10⁻⁵ mol) was dissolved in 47 mL DI water.

α,α-Trehalose Solution—α,α-Trehalose, 4.004 g was dissolved in 40 mL DI water to make a 10% solution.

Peptide Solution, IMP485 2 mM—The peptide IMP485 (0.0020 g, 1.52 μmol) was dissolved in 762 μL of 2 mM acetate buffer. The pH was 2.48 (the peptide was lyophilized as the TFA salt). The pH of the peptide solution was adjusted to pH 4.56 by the addition of 4.1 μL of 1 M NaOH.

Ascorbic Acid Solution 5 mg/mL—Ascorbic acid, 0.0262 g (1.49×10⁻⁴ mol) was dissolved in 5.24 mL DI water.

### Formulation of Peptide Kit

The peptide, 20 μL (40 nmol) was mixed with 12 μL (24 nmol) of Al, 100 μL of trehalose, 20 μL (0.1 mg) ascorbic acid and 900 μL of DI water in a 3 mL lyophilization vial. The final pH of the solution was about pH 4.0. The vial was frozen, lyophilized and sealed under vacuum. Ten and 20 nmol kits have also been made. These kits are made the same as the 40 nmol kits keeping the peptide to Al³⁺ ratio of 1 peptide to 0.6 Al³⁺ but formulated in 2 mL vials with a total fill of 0.5 mL.

Purification of ¹⁸F⁻

The crude ¹⁸F⁻ was received in 2 mL of DI water in a syringe. The syringe was placed on a syringe pump and the liquid pushed through a Waters CM cartridge followed by a QMA cartridge. Both cartridges were washed with 10 mL DI water. A sterile disposable three way valve between the two cartridges was switched and 1 mL commercial sterile saline was pushed through the QMA cartridge in 200 μL fractions. The second fraction usually contains ˜80% of the ¹⁸F⁻ regardless of the amount of ¹⁸F⁻ applied (10-300 mCi loads were tested).

We alternatively use commercial ¹⁸F⁻ in saline, which has been purified on a QMA cartridge. This is a concentrated version of the commercial bone imaging agent so it is readily available and used in humans. The activity is supplied in 200 μL in a 0.5 mL tuberculin syringe.

### Radiolabeling

The peptide was radiolabeled by adding ¹⁸F⁻ in 200 μL saline to the lyophilized peptide in a crimp sealed vial and then heating the solution to 90-110° C. for 15 min. The peptide was purified by adding 800 mL of DI water in a 1 mL syringe to the reaction vial, removing the liquid with the 1 mL syringe and applying the liquid to a Waters HLB column (l cc, 30 mg). The HLB column was placed on a crimp sealed 5 mL vial and the liquid was drawn into the vial under vacuum supplied by a remote (using a sterile disposable line) 10 mL syringe. The reaction vial was washed with two one mL aliquots of DI water, which were also drawn through the column. The column was then washed with 1 mL more of DI water. The column was then moved to a vial containing buffered lyophilized ascorbic acid (˜pH 5.5, 15 mg). The radiolabeled product was eluted with three 200 μL portions of 1:1 EtOH/DI water. The yield was determined by measuring the activity on the HLB cartridge, in the reaction vial, in the water wash and in the product vial to get the percent yield.

Adding ethanol to the radiolabeling reaction can increase the labeling yield. A 20 nmol kit can be reconstituted with a mixture of 200 μL ¹⁸F⁻ in saline and 200 μL ethanol. The solution is then heated to 110° C. in the crimp sealed vial for 16 min. After heating, 0.8 mL of water was added to the reaction vial and the activity was removed with a syringe and placed in a dilution vial containing 2 mL of DI water. The reaction vial was washed with 2×1 mL DI water and each wash was added to the dilution vial. The solution in the dilution vial was applied to the HLB column in 1-mL aliquots. The column and the dilution vial were then washed with 2×1-mL water. The radiolabeled peptide was then eluted from the column with 3×200 μL of 1:1 ethanol/water in fractions. The peptide can be labeled in good yield and up to 4,100 Ci/mmol specific activity using this method.

The yield for this kit and label as described was 80-90% when labeled with 1.0 mCi of ¹⁸F⁻. When higher doses of ¹⁸F⁻ (˜100 mCi) were used the yield dropped. However if ethanol is added to the labeling mixture the yield goes up. If the peptides are diluted too much in saline the yields will drop. The labeling is also very sensitive to pH. For our peptide with this ligand we have found that the optimal pH for the final formulation was pH 4.0±0.2.

The purified radiolabeled peptide in 50 μL 1:1 EtOH/H₂O was mixed with 150 μL of human serum and placed in the HPLC autosampler heated to 37° C. and analyzed by RP-HPLC. No detectable ¹⁸F above background at the void volume was observed even after 4 h.

TABLE 15
IMP 485 Labeling

| | | | | | | Activ- | | |
|---|---|---|---|---|---|---|---|---|
| | | | | | | ity | | Spe- |
| | | | | | Activ- | of | | cific |
| | | | Vol- | Vol- | ity | iso- | | Activ- |
| | | nmol | ume | ume | at | lated | | ity |
| Vial #/ | | of | Saline | EtOH | start | product | % | Ci/ |
| Peptide | | Peptide | μL | μL | mCi | mCi | Yield | mmol |
| 1. | IMP485 | 10 | 100 | 0 | 20.0 | 7.10 | 62 | |
| 2. | IMP485 | 10 | 50 | 50 | 19.4 | 9.43 | 78 | |
| 3. | IMP485 | 10 | 200 | 0 | 19.07 | 5.05 | 38 | |
| 4. | IMP485 | 20 | 100 | 100 | 37.3 | 22.3 | 80 | |
| 5. | IMP485 | 10 | 100 | 0 | 45.7 | 16.2 | 42 | |
| 6. | IMP485 | 20 | 200 | 200 | 175.6 | 82.7 | 58 | 4135 |

### Synthesis and Radiolabeling of IMP486: Al-OH(IMP485)

IMP485 (21.5 mg, 0.016 mmol) was dissolved in 1 mL of 2 mM NaOAc, pH 4.4 and treated with AlCl₃.6H₂O (13.2 mg, 0.055 mmol). The pH was adjusted to 4.5-5.0 and the reaction mixture was refluxed for 15 minutes. The crude mixture was purified by preparative RP-HPLC to yield a white solid (11.8 mg).

The pre-filled Al(NOTA) complex (IMP486) was also radiolabeled in excellent yield after formulating into lyophilized kits. The labeling yields with IMP486 (Table 16) were as good as or better than IMP485 kits (Table 15) when labeled in saline. This high efficiency of radiolabeling with chelator preloaded with aluminum was not observed with any of the other Al(NOTA) complexes tested (data not shown). The equivalency of labeling in saline and in 1:1 ethanol/water the labeling yields was also not observed with other chelating moieties (not shown).

TABLE 16
IMP486 Labeling

| | | | | | | Activ- | | |
|---|---|---|---|---|---|---|---|---|
| | | | | | Activ- | ity | | Specific |
| | | | | | ity | of | | Activ- |
| | Vol- | | Vol- | | at | isolated | | ity |
| Peptide | ume | | ume | | start | product | % | Ci/ |
| 20 nmol | Saline | | EtOH | | mCi | mCi | Yield | mmol |
| IMP486 | 100 | μL | 0 | | 46 | 28 | 76 | 2800 |
| IMP485 | 100 | μL | 100 | μL | 41 | 25 | 83 | |
| IMP486 | 100 | μL | 100 | μL | 43 | 22 | 81 | |

| | | | | | | |
|---|---|---|---|---|---|---|
| IMP486 | 200 | μL | 0 | 42 | 18 | 73 |

### Effect of Bulking Agents

An experiment was performed to compare yield using IMP485 kits (40 nmol) with different bulking agents. The peptide was labeled with 2 mCi of ¹⁸F⁻ from the same batch of ¹⁸F⁻ in 200 microliters saline. The bulking agents were introduced in water at a concentration of 5% by weight, with a dose of 200 microliters/vial. We tested sorbitol, trehalose, sucrose, mannitol and glycine as bulking agents. Results are shown in Table 17.

TABLE 17
Effects of Bulking Agents on Radiolabeling Yield

| Bulking Agent | Activity mCi | Yield % |
|---|---|---|
| Sorbitol | 2.17 | 82.9 |
| Glycine | 2.17 | 41.5 |
| Mannitol | 2.11 | 81.8 |
| Sucrose | 2.11 | 66.1 |
| Trehalose | 2.10 | 81.3 |

Sorbitol, mannitol and trehalose all gave radiolabeled product in the same yield. The sucrose kit and the glycine kit both had significantly lower yields. Trehalose was formulated into IMP485 kits at concentrations ranging from 2.5% to 50% by weight when reconstituted in 200 μL. The same radiolabeling yield, ˜83%, was obtained for all concentrations, indicating that the ¹⁸F-radiolabeling of IMP485 was not sensitive to the concentration of the trehalose bulking agent. IMP 485 kits were formulated and stored at 2-8° C. under nitrogen for up to three days before lyophilization to assess the impact of lyophilization delays on the radiolabeling. The radiolabeling experiments indicated that yields were all ˜80% at time zero, and with 1, 2, and 3 days of delay before lyophilization.

Ascorbic or gentisic acid often are added to radiopharmaceuticals during preparation to minimize radiolysis. When IMP485 (20 nmol) was formulated with 0.1, 0.5 and 1.0 mg of ascorbic acid at pH 4.1-4.2 and labeled with ¹⁸F⁻ in 200 μL saline, final yields were 51, 31 and 13% isolated yields, respectively, suggesting 0.1 mg of ascorbic acid was the maximum amount that could be included in the formulation without reducing yields. Formulations containing gentisic acid did not label well. Ascorbic acid was also included in vials used to isolate the HLB purified product as an additional means of ensuring stability post-labeling. The IMP485 to Al³⁺ ratio appeared to be optimal at 1:0.6, but good yields were obtained from 1:0.5 of up to a ratio of 1:1. The radiolabeling reaction was also sensitive to peptide concentration, with good yields obtained at concentrations of 1×10⁻⁴ M and higher.

### Effect of pH on Radiolabeling

The effect of pH on radiolabeling of IMP485 is shown in Table 18. The efficiency of labeling was pH sensitive and decreased at either higher or lower pH relative to the optimal pH of about 4.0.

TABLE 18
Effect of pH on IMP485 Radiolabeling Efficiency.

| pH | Yield % |
|---|---|
| 3.27 | 33 |
| 3.53 | 61 |
| 3.84 | 85 |
| 3.99 | 88 |
| 4.21 | 89 |
| 4.49 | 80 |
| 5.07 | 14 |

Collectively, these studies led to a final formulated kit that contained 0.5 mL of a sterile solution with 20 nmol IMP485, 12 nmoles Al³⁻, 0.1 mg ascorbic acid, and 10 mg trehalose adjusted to 4.0±0.2, which was then lyophilized

### Biodistribution

Biodistribution studies were performed in Taconic nude mice bearing subcutaneous LS174T tumor xenografts.

Al¹⁸F(IMP485) alone: Mice bearing sc LS174T xenografts were injected with Al¹⁸F(IMP485) (28 μCi, 5.2×10¹ mol, 100 μL, iv). Mice were necropsied at 1 and 3 h post injection, 6 mice per time point.

TF2+Al¹⁸F(IMP485) Pretargeting at 20:1 bsMab to peptide ratio: Mice bearing sc LS174T xenografts were injected with TF2 (163.2 μg, 1.03×10⁻⁹ mol, iv) and allowed 16.3 h for clearance before injecting Al¹⁸F(IMP485) (28 μCi, 5.2×10¹ mol, 100 μL, iv). Mice were necropsied at 1 and 3 h post injection, 7 mice per time point.

TABLE 19
Biodistribution of TF2 pretargeted Al¹⁸F(IMP485) or
Al¹⁸F(IMP485) alone at 1 and 3 h after peptide injection
in nude mice bearing LS174T human colonic cancer xenografts.

| | Percent-injected dose per gram tissue (mean ± SD; N = 7) | | | |
|---|---|---|---|---|
| | TF2 pregargeted Al¹⁸F(IMP485) | | Al¹⁸F(IMP485) alone | |
| Tissue | 1 h | 3 h | 1 h | 3 h |
| Tumor | 28.09 ± 4.55 | 26.52 ± 5.97 | 0.32 ± 0.11 | 0.09 ± 0.02 |
| Liver | 0.24 ± 0.04 | 0.14 ± 0.02 | 0.18 ± 0.03 | 0.10 ± 0.05 |
| Spleen | 0.25 ± 0.11 | 0.25 ± 0.11 | 0.21 ± 0.18 | 0.07 ± 0.01 |
| Kidney | 3.19 ± 0.73 | 2.22 ± 0.22 | 3.33 ± 0.56 | 2.27 ± 0.29 |
| Lung | 0.54 ± 0.15 | 0.24 ± 0.06 | 0.24 ± 0.05 | 0.07 ± 0.02 |
| Blood | 0.28 ± 0.07 | 0.02 ± 0.01 | 0.17 ± 0.06 | 0.09 ± 0.01 |
| Stomach | 0.53 ± 1.18 | 0.03 ± 0.02 | 0.13 ± 0.11 | 0.04 ± 0.02 |
| Sm. Int. | 0.69 ± 0.88 | 0.07 ± 0.03 | 0.40 ± 0.13 | 0.14 ± 0.03 |
| Lg. Int. | 0.10 ± 0.03 | 0.35 ± 0.16 | 0.08 ± 0.02 | 0.71 ± 0.22 |
| Muscle | 0.14 ± 0.10 | 0.03 ± 0.01 | 0.11 ± 0.08 | 0.01 ± 0.01 |
| Scapula | 0.5 ± 0.45 | 0.06 ± 0.02 | 0.11 ± 0.02 | 0.03 ± 0.01 |

### Synthesis of IMP492 or Al¹⁹F(IMP485)

IMP485 (16.5 mg, 0.013 mmol) was dissolved in 1 mL of 2 mM NaOAc, pH 4.43, 0.5 mL ethanol and treated with AlF₃.3H₂O (2.5 mg, 0.018 mmol). The pH was adjusted to 4.5-5.0 and the reaction mixture was refluxed for 15 minutes. On cooling the pH was once again raised to 4.5-5.0 and the reaction mixture refluxed for 15 minutes. The crude was purified by preparative RP-HPLC to yield a white solid (10.3 mg).

### Synthesis of IMP490

(SEQ ID NO: 22)
NODA-MPAA-D-Phe-Cys-Phe-D-Trp-Lys-Thr-Cys-Throl

The peptide was synthesized on threoninol resin with the amino acids added in the following order: Fmoc-Cys(Trt)-OH, Fmoc-Thr(But)-OH, Fmoc-Lys(Boc)-OH, Fmoc-D-Trp(Boc)-OH, Fmoc-Phe-OH, Fmoc-Cys(Trt)-OH, Fmoc-D-Phe-OH and (tBu)₂NODA-MPAA. The peptide was then cleaved and purified by preparative RP-HPLC. The peptide was cyclized by treatment of the bis-thiol peptide with DMSO.

### Synthesis of IMP491 or Al¹⁹F(IMP490)

The Al¹⁹F(IMP490) was prepared as described above (IMP492) to produce the desired peptide after HPLC purification.

### Synthesis of IMP493

(SEQ ID NO: 23)
NODA-MPAA-(PEG)₄-Gln-Trp-Ala-Val-Gly-His-Leu-Met-
NH₂

The peptide was synthesized on Sieber amide resin with the amino acids added in the following order: Fmoc-Met-OH, Fmoc-Leu-OH, Fmoc-His(Trt)-OH, Fmoc-Gly-OH, Fmoc-Val-OH, Fmoc-Ala-OH, Fmoc-Trp(Boc)-OH, Fmoc-Gln(Trt)-OH, Fmoc-NH-(PEG)₃-COOH and (tBu)₂NODA-MPAA. The peptide was then cleaved and purified by preparative RP-HPLC.

The affinity of the Al¹⁹F complex of IMP493 was EC₅₀=183 nm versus EC₅₀=59 nm for ¹²⁵I-bombesin. The IMP493 kit radiolabeled with ˜100 MBq of ¹⁸F⁻ had a 70% yield. Radiolabeling IMP490 with 100 MBq of ¹⁸F⁻ resulted in 80% yield, which was reduced to 65% when 2.11 GBq ¹⁸F⁻ was used. The peptide is eluted as a single radiolabeled peak at 15.4 min using HPLC (not shown).

### Synthesis of IMP494 or Al¹⁹F(IMP493)

The Al¹⁹F(IMP493) was prepared as described above (IMP492) to produce the desired peptide after HPLC purification.

### Example 26

### Other Prosthetic Group Labeling Methods Using Al¹⁸F

In certain embodiments, the aluminum fluoride labeling method may be performed using prosthetic group labeling methods for molecules that are sensitive to heat. Prosthetic group conjugation may be carried out at lower temperatures for heat-sensitive molecules.

The prosthetic group NOTA is labeled with ¹⁸F as described above and then it is attached to the targeting molecule. In one non-limiting example, this is performed with an aldehyde NOTA that is then attached to an amino-oxy compound on a targeting molecule. Alternatively an amino-oxy maleimide is reacted with the aldehyde and then the maleimide is attached to a cysteine on a targeting molecule (Toyokuni et al., 2003, Bioconj Chem 14:1253).

In another alternative, the AlF-chelator complexes are attached to targeting molecules through click chemistry. The ligands are first labeled with Al¹⁸F as discussed above. The Al¹⁸F-chelate is then conjugated to a targeting molecule through a click chemistry reaction. For example, an alkyne NOTA is labeled according to Marik and Stucliffe (2006, Tetrahedron Lett 47:6681) and conjugated to an azide containing targeting agent.

### Radiolabeling of Kits With ¹⁸F⁻ in Saline

The ¹⁸F (0.01 mCi or higher) is received in 200 μL of saline in a 0.5 mL syringe and the solution is mixed with 200 μL of ethanol and injected into a lyophilized kit as described above. The solution is heated in the crimp sealed container at 100-110° C. for 15 min. The solution is diluted with 3 mL water and eluted through an HLB cartridge. The reaction vial and the cartridge are washed with 2×1 mL portions of water and then the product is eluted into a vial containing buffered ascorbic acid using 1:1 ethanol water in 0.5 mL fractions. Some of the ethanol may be evaporated off under a stream of inert gas. The solution is then diluted in saline and passed through a 0.2 μm sterile filter prior to injection.

### Example 27

### Maleimide Conjugates of NOTA for ¹⁸ F-Labeling

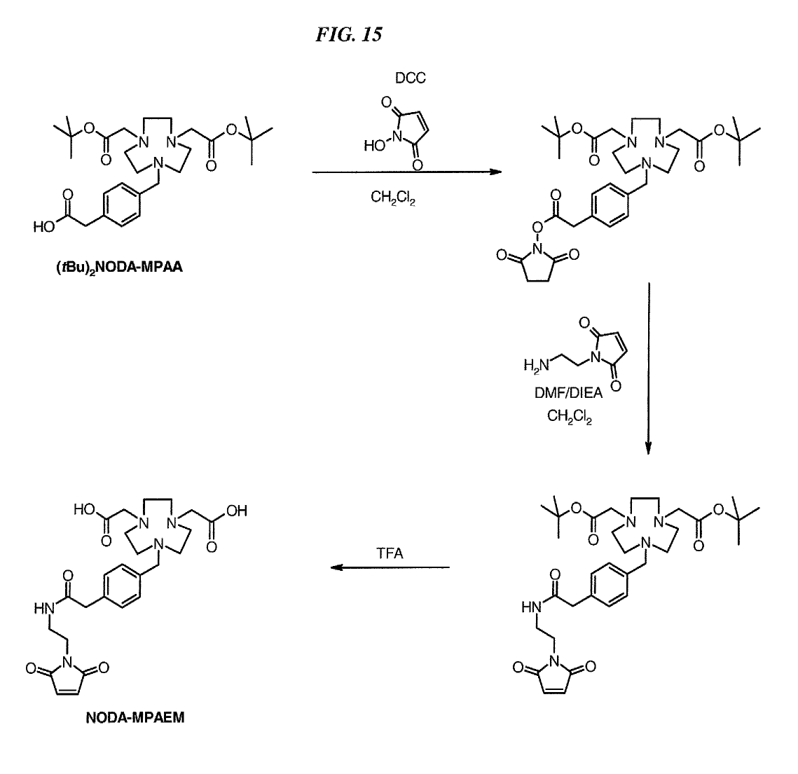

The Examples above describe a novel method of ¹⁸F-labeling, which captures a ([¹⁸F]AlF)²⁺ complex, using a NOTA-derived ligand bound on a peptide. These labeled peptides were stable in vivo and retained their binding abilities (McBride et al., 2009, J. Nucl. Med. 50, 991-998; McBride et al., 2010, Bioconjug. Chem. 21, 1331-1340; Laverman et al., 2010, J. Nucl. Med. 51, 454-461; McBride et al. 2011, J. Nucl. Med. 52 (Suppl. 1), 313-314P (abstract 1489)). Although this procedure allows peptides to be radiofluorinated in one simple step within 30 min, it requires agents to be heated to ˜100° C., which is unsuitable for most proteins and some peptides. We and others have found that an aromatic group attached to one of the nitrogen atoms of the triazacylcononane ring of NOTA can enhance the yield for the ([¹⁸F]AlF)²⁺ complexation compared to some alkyl and carboxyl substituents (D'Souza et al., 2011, Bioconjug. Chem. 1793-1803 ; McBride et al., 2010, Bioconjug. Chem. 21, 1331-1340; Shetty et al. 2011, Chem. Comm. DOI: 10.1039/ciccl3151f). In the present Example, we explored the potential for labeling heat-labile compounds with ([¹⁸F]AlF)²⁺, using a new ([¹⁸F]AlF)²⁺-binding ligand that contains NOTA attached to a methyl phenylacetic acid group (MPAA). This was conjugated to N-(2-aminoethyl)maleimide (N-AEM) to form NOTA-MPAEM. (Details of the synthesis are shown in FIG. 15.) The NOTA-MPAEM was labeled with ([¹⁸F]AlF)²⁺ at 105° C. in 49-82% yield and conjugated at room temperature to an antibody Fab' fragment in 69-80% yield (total time ˜50 min) and with retention of immunoreactivity. These data indicate that the rapid and simple [Al¹⁸F]-labeling method can be easily adapted for preparing heat-sensitive compounds with ¹⁸F quickly and in high yields.

Synthesis of Bis-t-butyl-NOTA-MPAA NHS ester: (tBu)₂NOTA-MPAA NHS ester

To a solution of (tBu)₂NOTA-MPAA (175.7 mg, 0.347 mmol) in CH₂Cl₂ (5 mL) was added 347 μL (0.347 mmol) DCC (1 M in CH₂Cl₂), 42.5 mg (0.392 mmol) N-hydroxysuccinimide (NHS), and 20 μL N,N-diisopropylethylamine (DIEA). After 3 h DCU was filtered off and solvent evaporated. The crude mixture was purified by flash chromatography on (230-400 mesh) silica gel (CH₂Cl₂:MeOH, 100:0 to 80:20) to yield (128.3 mg, 61.3%) of the NHS ester. The HRMS (ESI) calculated for C₃₁H₄₆N₄O₈ (M+H)⁺ was 603.3388, observed was 603.3395.

Synthesis of NOTA-MPAEM: (MPAEM=methyl phenyl acetamido ethyl maleimide)

To a solution of (tBu)₂NOTA-MPAA NHS ester (128.3 mg, 0.213 mmol) in CH₂Cl₂ (5 mL) was added a solution of 52.6 mg (0.207 mmol) N-(2-aminoethyl) maleimide trifluoroacetate salt in 250 μL DMF and 20 μL DIEA. After 3 h the solvent was evaporated and the concentrate was treated with 2 mL TFA. The crude product was diluted with water and purified by preparative RP-HPLC to yield (49.4 mg, 45%) of the desired product. HRMS (ESI) calculated for C₂₅H₃₃N₅O₇ (M+H)⁺ was 516.2453, observed was 516.2452.

### ¹⁸ F-Labeling of NOTA-MPAEM

The NOTA-MPAEM ligand (20 nmol; 10 μL), dissolved in 2 mM sodium acetate (pH 4), was mixed with AlCl₃ (5 μL of 2 mM solution in 2 mM acetate buffer, 200 μL of ¹⁸F⁻ (0.73 and 1.56 GBq) in saline, and 200 μL of acetonitrile. After heating at 105-109° C. for 15-20 min, 800 μL of deionized (DI) water was added to the reaction solution, and the entire contents removed to a vial (dilution vial) containing 1 mL of deionized (DI) water. The reaction vial was washed with 2×1 mL DI water and added to the dilution vial. The crude product was then passed through a 1-mL HLB column, which was washed with 2×1 mL fractions of DI water. The labeled product was eluted from the column using 3×200 μL of 1:1 EtOH/water.

Conjugation of Al¹⁸F(NOTA-MPAEM) to hMN-14 Fab′

Fab⁺ fragments of humanized MN-14 anti-CEACAM5 IgG (labetuzumab) were prepared by pepsin digestion, followed by TCEP (Tris(2-carboxyethyl)phosphine) reduction, and then formulated into a lyophilized kit containing 1 mg (20 nmol) of the Fab′ (2.4 thiols/Fab′) in 5% trehalose and 0.025 M sodium acetate, pH 6.72. The kit was reconstituted with 0.1 mL PBS, pH 7.01, and mixed with the Al¹⁸F(NOTA-MPAEM) (600 μL 1:1 EtOH/H₂O). After incubating for 10 min at room temperature, the product was purified on a 3-mL SEPHADEX G50-80 spin column in a 0.1 M, pH 6.5 sodium acetate buffer (5 min). The isolated yield was calculated by dividing the amount of activity in the eluent by the total activity in the eluent and the activity on the column.

Immunoreactivity of the purified product was analyzed by adding an excess of CEA and separating on SE-HPLC, comparing to a profile of the product alone. The product was also analyzed by RP-HPLC to assess percent-unbound Al¹⁸F(NOTA-MPAEM).

^{99m}Tc-CEA-Scan®

A CEA-SCAN® kit containing 1.2 mg of IMMU-4, a murine anti-CEACAM5 Fab′ (anti-CEA, 2.4×10⁻² μmol), was labeled with 453 MBq ^{99m}TcO₄⁻Na⁻¹ in 1 mL saline according to manufacturer's instructions and used without further purification.

### Animal Study

Nude mice were inoculated subcutaneously with CaPan-1 human pancreatic adenocarcinoma (ATCC Accession No. HTB-79, Manassas, Va.). When tumors were visible, the animals were injected intravenously with 100 μL of the radiolabeled Fab′. The Al¹⁸F(NOTA-MPAES)-hMN-14 Fab′ was diluted in saline to 3.7 MBq/100 μL containing ˜2.8 μg of Fab′. A ^{99m}Tc-IMMU-4 Fab' aliquot (16.9 MBq) was removed and diluted with saline (0.85 MBq/100 μL containing ˜2.8 μg of Fab′). The animals were necropsied at 3 h post injection, tissues and tumors removed, weighed, and counted by gamma scintillation, together with standards prepared from the injected products. The data are expressed as percent injected dose per gram.

### Results

### Synthesis and Reagent Preparation

The NOTA-MPAEM was produced as shown in FIG. 15, where the (tBu)₂NOTA-MPAA was coupled to 2-aminoethyl-maleimide and then deprotected to form the desired product. The crude product was diluted with water and purified by preparative RP-HPLC to yield (49.4 mg, 45%) of the desired product [HRMS (ESI) calculated for C₂₅H₃₃N₅O₇ (M+H)⁺ 516.2453, found 516.2452].

### Radiolabeling

The NOTA-MPAEM (20 nmol) was mixed with 10 nmol of Al³⁺ and labeled with 0.73 GBq and 1.56 GBq of ¹⁸F⁻ in saline. After SPE purification, the isolated yields of Al'⁸F(NOTA-MPAEM) were 82% and 49%, respectively, with a synthesis time of about 30 min. The Al¹⁸F(NOTA-MPAES)-hMN-14 Fab' conjugate was isolated in 74% and 80% yields after spin-column purification for the low and high dose protein labeling, respectively. The total process was completed within 50 min. The specific activity for the purified Al¹⁸F(NOTA-MPAES)-hMN-14 Fab' was 19.5 GBq/μmol for the high-dose label and 10.9 GBq/μmol for the low dose label.

SE-HPLC analysis of the labeled protein for the 0.74-GBq run showed the ¹⁸F-labeled Fab′ as a single peak and all of the activity shifted when excess CEA was added (not shown). RP-HPLC analysis on a C4 column showed the labeled maleimide standard eluting at 7.5 min, while the purified ¹⁸F-protein eluted at 16.6 min (not shown). There was no unbound Al¹⁸F(NOTA-MPAEM) in the spin-column purified product.

### Serum Stability

The Al¹⁸F(NOTA-MPAES)-hMN-14 Fab′ was mixed with fresh human serum and incubated at 37° C. SE-HPLC analysis over a 3-h period, with and without CEA showed that the product was stable and retained binding to CEA (not shown).

### Biodistribution

The biodistribution of the Al¹⁸F(NOTA-MPAES)-hMN-14 Fab′ and the ^{99m}Tc-IMMU-4 murine Fab′ was assessed in nude mice bearing Capan-1 pancreatic cancer xenografts. At 3 h post-injection, both agents showed an expected elevated uptake in the kidneys, since Fab′ is renally filtered from the blood (Table 20). The [Al¹⁸F]-Fab′ concentration in the blood was significantly (P<0.0001) lower than the ^{99m}Tc-Fab′, with a correspondingly elevated uptake in the liver and spleen. The faster blood clearance of the [Al¹⁸F]-Fab′ likely contributed to the lower tumor uptake as compared to the ^{99m}Tc-Fab′ (2.8±0.3 vs. 6.8±0.7, respectively), but it also resulted in a more favorable tumor/blood ratio for the fluorinated Fab′ (5.9±1.3 vs. 0.9±0.1, respectively). Bone uptake for both products was similar, suggesting the Al¹⁸F(NOTA) was tightly held by the Fab′.

TABLE 20
Biodistribution of Al¹⁸F(NOTA-MPAES)-hMN-14 Fab′ and ^{99m}Tc-IMMU-4
Fab′ at 3 h after injection with 0.37 MBq (~3 μg) of each conjugate
in nude mice bearing Capan-1 human pancreatic cancer xenografts (N = 6).

| | Al¹⁸F(NOTA-MPAES)- | | ^{99m}Tc CEA Scan | |
|---|---|---|---|---|
| | hMN-14 Fab′ | | IMMU-4 Fab′ | |
| Tissue | % ID/g | T/NT | % ID/g | T/NT |
| Capan-1 | 2.8 ± 0.3 | — | 6.8 ± 0.7 | — |
| (weight ± SD) | (0.22 ± 0.08 g) | | (0.16 ± 0.05 g) | |
| Liver | 17.5 ± 3.8 | 0.2 ± 0.04 | 4.6 ± 0.4 | 1.5 ± 0.1 |
| Spleen | 11.3 ± 1.6 | 0.3 ± 0.04 | 3.5 ± 0.5 | 2.0 ± 0.3 |
| Kidney | 216 ± 30.9 | 0.0 ± 0.0 | 183 ± 22.5 | 0.04 ± 0.01 |
| Lung | 4.2 ± 1.6 | 0.8 ± 0.5 | 4.4 ± 0.8 | 1.6 ± 0.3 |
| Blood | 0.5 ± 0.1 | 5.9 ± 1.3 | 7.6 ± 0.9 | 0.9 ± 0.1 |
| Stomach | 0.6 ± 0.1 | 4.7 ± 1.2 | 2.4 ± 0.3 | 2.9 ± 0.4 |
| Sm. Int. | 2.2 ± 0.2 | 1.3 ± 0.1 | 3.4 ± 0.4 | 2.0 ± 0.2 |
| Lg. Int. | 1.1 ± 0.4 | 2.8 ± 0.7 | 5.2 ± 1.0 | 1.3 ± 0.3 |
| Muscle | 0.4 ± 0.1 | 6.7 ± 1.3 | 1.1 ± 0.2 | 6.1 ± 1.0 |
| Scapula | 1.6 ± 0.4 | 1.8 ± 0.4 | 2.0 ± 0.2 | 3.4 ± 0.5 |

### Discussion

We prepared a simple NOTA-MPAEM ligand for attachment to thiols on temperature-sensitive proteins or other molecules bearing a sulfhydryl group. To avoid exposing the heat-labile compound to high temperatures, the NOTA-MPAEM was first mixed with Al³⁺ and ¹⁸F⁻ in saline and heated at 100-115° C. for 15 min to form the Al¹⁸F(NOTA-MPAEM) intermediate. This intermediate was rapidly purified by SPE in 49-82% isolated yield (67.7±13.0%, n=5), depending on the amount of activity added to a fixed amount (20 nmol) of the NOTA-MPAEM. The Al¹⁸F(NOTA-MPAEM) was then efficiently (69-80% isolated yield, 74.3±5.5, n=3) coupled to a reduced Fab' in 10-15 min, using a spin column gel filtration procedure to isolate the radiolabeled protein, in this case an antibody Fab′ fragment. The entire two-step process was completed in ˜50 min, and the labeled product retained its molecular integrity and immunoreactivity. Thus, the feasibility of extending the simplicity of the [Al¹⁸F]-labeling procedure to heat-sensitive compounds was established.

The [Al¹⁸F]-ligand complex has been shown to be very stable in serum in vitro, and in animal testing, minimal bone uptake is seen (McBride et al., 2009, J. Nucl. Med. 50, 991-998; D'Souza et al., 201 la, J. Nucl. Med. 52 (Suppl. 1), 171P (abstract 577)). In this series of studies, ¹⁸F associated with the NOTA-MPAEM compound conjugated to a Fab' was stable in serum in vitro, and the conjugate retained binding to CEA. When injected into nude mice, there was selective localization in the tumor, providing a ˜6:1 tumor/blood ratio. Bone uptake was similar for the Al¹⁸F(NOTA-MPAES)-hMN-14 Fab′ and the ^{99m}Tc-IMMU-4 murine Fab′, again reflecting in vivo stability of the ¹⁸F or Al¹⁸F complex. However, [Al¹⁸F]-Fab′ hepatic and splenic uptake was higher as compared to the ^{99m}Tc-IMMU-4. The specific NOTA derivative can be modified in different ways to accommodate conjugation to other reactive sites on peptides or proteins. However, use of this particular derivative showed that the Al¹⁸F-labeling procedure can be adapted for use with heat-labile compounds.

### Conclusions

NOTA-MPAEM was labeled rapidly with ¹⁸F in saline and then conjugated to the immunoglobulin Fab' protein in high yield. The labeling method uses only inexpensive disposable purification columns, and while not requiring an automated device to perform the labeling and purification, it can be easily adapted to such systems. Thus, the NOTA-MPAEM derivative established that this or other NOTA-containing derivatives can extend the capability of facile ([¹⁸F]AlF)²⁺ fluorination to heat-labile compounds.

### Example 28

### Improved ¹⁸ F-Labeling of NOTA-Octreotide

The aim of this study was to further improve the rapid one-step method for ¹⁸F-labeling of NOTA-conjugated octreotide. Octreotide was conjugated with a NOTA ligand and was labeled with ¹⁸F in a single- step, one-pot method. Aluminum (Al³⁺) was added to ¹⁸F⁻ and the AlF² was incorporated into NOTA-octreotide, as described in the Examples above. The labeling procedure was optimized with regard to aluminum:NOTA ratio, ionic strength and temperature. Radiochemical yield and specific activity were determined.

Under optimized conditions, NOTA-octreotide was labeled with Al¹⁸F in a single step with 98% yield. The radiolabeling, including purification, was performed in 45 min. Optimal labeling yield was observed with Al:NOTA ratios around 1:20. Lower ratios led to decreased labeling efficiency. Labeling efficiencies in the presence of 0%, 25%, 50%, 67% and 80% acetonitrile in Na-acetate pH 4.1 were 36%, 43%, 49%, 70% and 98%, respectively, indicating that increasing concentrations of the organic solvent considerably improved labeling efficiency. Similar results were obtained in the presence of ethanol, DMF and THF. Labeling in the presence of DMSO failed. Labeling efficiencies in 80% MeCN at 40° C., 50° C. and 60° C. were 34%, 65%, 83%, respectively. Labeling efficiency was >98% at 80° C. and 100° C. Specific activity of the ¹⁸F-labeled peptide was higher than 45,000 GBq/mmol.

Optimal ¹⁸F-labeling of NOTA-octreotide with Al¹⁸F was performed at 80-100° C. in Na-acetate buffer with 80% (v/v) acetonitrile and a Al:NOTA ratio between 1:20 and 1:50. Labeling efficiency was typically >98%. Since labeling efficiency at 60° C. was 83%, this method may also allow ¹⁸F-labeling of temperature-sensitive biomolecules such as proteins and antibody fragments. These conditions allow routine ¹⁸F-labeling of peptides without the need for purification prior to administration and PET imaging.

### Example 29

### Functionalized Triazacyclononane Ligands for Molecular Imaging

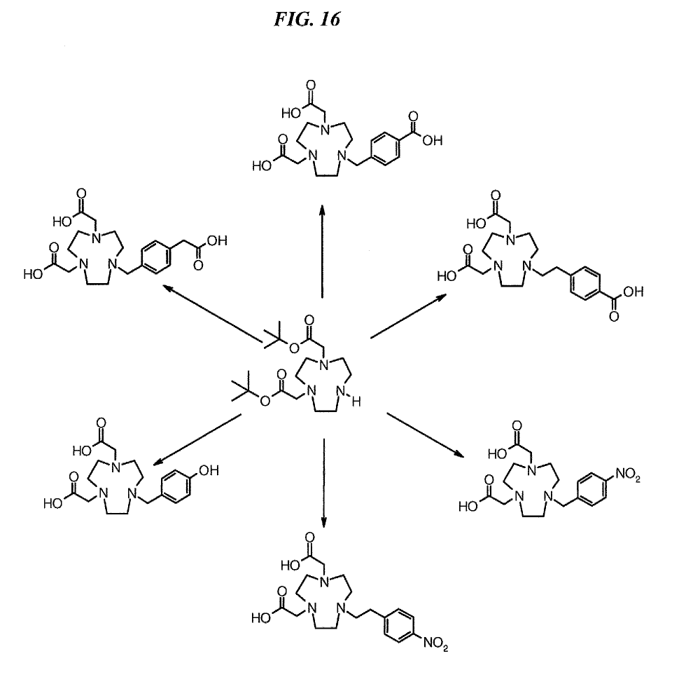

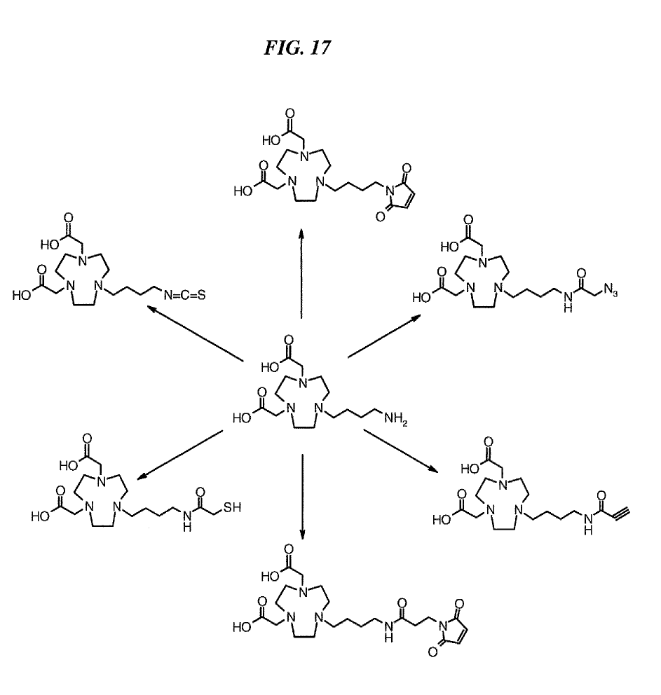

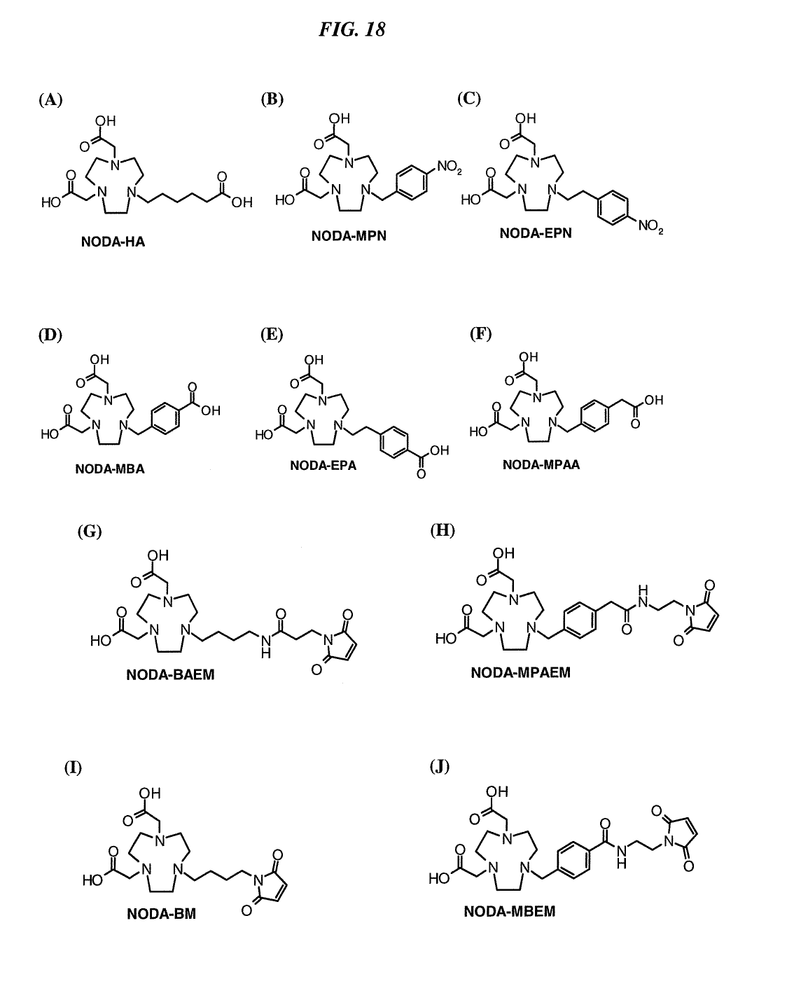

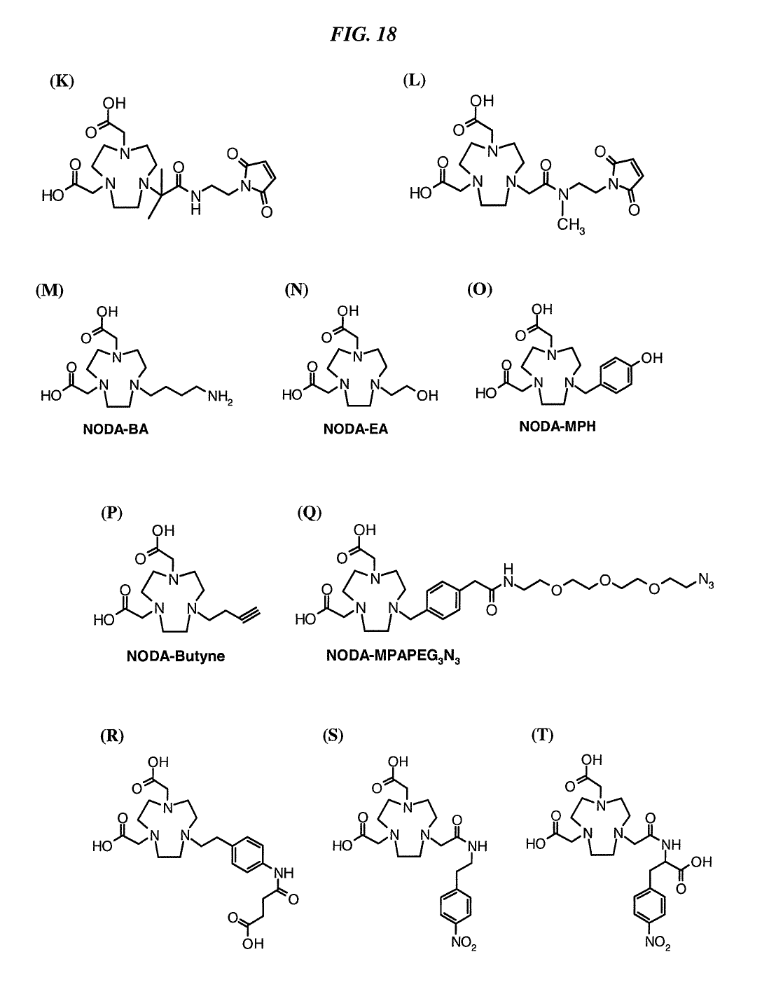

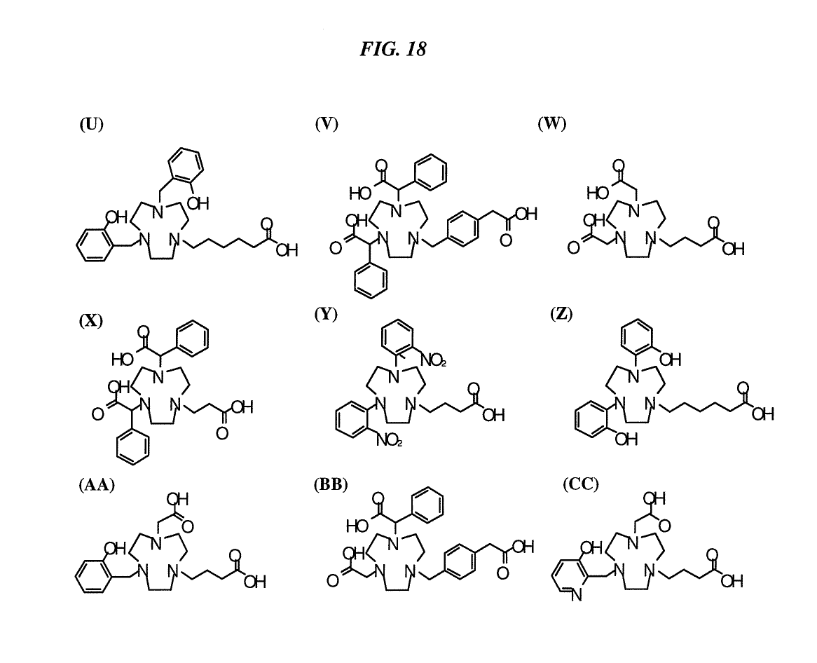

The present Example relates to synthesis and use of a new class of triazacyclonane derived ligands and their complexes useful for molecular imaging. Exemplary structures are shown in FIG. 16 to FIG. 18. The ligands may be functionalized with a ¹⁹F moiety selected from the group consisting of fluorinated alkyls, fluorinated acetates, fluoroalkyl phosphonates, fluoroanilines, trifluoromethyl anilines, and trifluoromethoxy anilines in an amount effective to provide a detectable ¹⁹F NMR signal. The complexation of these ligands with radioisotopic or paramagnetic cations renders them useful as diagnostic agents in nuclear medicine and magnetic resonance imaging (MRI). Preferably, the Al¹⁸F and ⁶⁸Ga complexes of these ligands are useful for PET imaging, while the ¹¹¹ In complexes can be used in SPECT imaging. Methods for conjugating these radiolabeled ligands to a targeting molecule like antibody, protein or peptide are also disclosed.

The disclosed bifunctional chelators (BFCs) can be radiolabeled with ¹¹¹In, ⁶⁸Ga, ⁶⁴Cu, ¹⁷⁷Lu, Al¹⁸F, ^{99m}Tc or ⁸⁶Y or complexed with a paramagnetic metal like manganese, iron, chromium or gadolinium, and subsequently attached to a targeting molecule (biomolecule). The labeled biomolecules can be used to image the hematological system, lymphatic reticuloendothelial system, nervous system, endocrine and exocrine system, skeletomuscular system, skin, pulmonary system, gastrointestinal system, reproductive system, immune system, cardiovascular system, urinary system, auditory or olfactory system or to image affected cells or tissues in various medical conditions.

### Synthesis of Bifunctional Chelators

### 2-{4-(carboxymethyl)-7-[2-(4-nitrophenyl)ethyl]-1,4,7-triazacyclononan-1-yl)acetic acid

### NOTA-EPN

To a solution of 4-nitrophenethyl bromide (104.5 mg, 0.45 mmol) in anhydrous CH₃CN at 0° C. was added dropwise over 20 min a solution of (tBu)₂NOTA (167.9 mg, 0.47 mmol) in CH₃CN (10 mL). After 1 h, anhydrous K₂CO₃ (238.9 mg, 1.73 mmol) was added to the reaction mixture and allowed to stir at room temperature overnight. Solvent was evaporated and the concentrate was acidified with 4 mL TFA. After 5 h, the reaction mixture was diluted with water and purified by preparative RP-HPLC to yield a pale yellow solid (60.8 mg, 32.8%). HRMS (ESI) calculated for C₁₈H₂₆N₄O₆ (M+H⁺ 395.1925; found 395.1925.

### 2-{4-(carboxymethyl)-7-[2-(4-nitrophenyl)methyl]-1,4,7-triazacyclononan-1-yl)acetic acid. NOTA-MPN

To a solution of 4-nitrobenzyl bromide (61.2 mg, 0.28 mmol) in anhydrous CH₃CN at 0° C. was added dropwise over 20 min a solution of (tBu)₂NOTA (103.6 mg, 0.29 mmol) in CH₃CN (10 mL). After 1 h, anhydrous K₂CO₃ (57.4 mg, 0.413 mmol) was added to the reaction mixture and allowed to stir at room temperature overnight. Solvent was evaporated and the concentrate was acidified with 3 mL TFA. After 5 h, the reaction mixture was diluted with water and purified by preparative RP-HPLC to yield a pale yellow solid (19.2 mg, 17.4%). HRMS (ESI) calculated for C₁₇H₂₄N₄O₆ (M+H)⁺ 381.1769; found 381.1774.

### 6-(4,7-bis{[(tert-butyl)oxycarbonyl]methyl}-1,4,7-triazacyclononan-1-yl)hexanoic acid. (tBu)₂NOTA-HA

To a solution of (tBu)₂NOTA (208.3 mg, 0.58 mmol) in 10 mL CH₃CN was added 6-bromohexanoic acid (147.3 mg, 0.755 mmol) and K₂CO₃ (144.5 mg, 1.05 mmol). The reaction flask was placed in a warm water-bath for 48 h. Solvent was evaporated and the concentrate was diluted with water and purified by preparative RP-HPLC to yield a white solid (138.5 mg, 50.1%). ESMS calculated for C₂₄H₄₅N₃O₆ (M+H)⁺ 472.3381; found 472.27.

### 4-[(4,7-bis{[(tert-butyl)oxycarbonyl]methyl}-1,4,7-triazacyclononan-1-yl) methyl]benzoic acid. (tBu)₂NOTA-MBA

To a solution of a-bromo-p-toluic acid (126.2 mg, 0. 59 mmol) in anhydrous CH₃CN was added dropwise over 20 min a solution of (tBu)₂NOTA (208 mg, 0.58 mmol) in CH₃CN (10 mL) and allowed to stir at room temperature for 48 h. Solvent was evaporated and the concentrate was diluted with water/DMF and purified by preparative RP-HPLC to yield a white solid (74.6 mg). HRMS (ESI) calculated for C₂₆H₄₁N₃O₆ (M+H)⁺ 492.3068; found 492.3071.

### 4-[(4,7-bis{[(tert-butyl)oxycarbonyl]methyl}-1,4,7-triazacyclononan-1-yl)ethyl]benzoic acid. (tBu)₂NOTA-EBA

To a solution of 4-(2-bromoethyl)benzoic acid (310.9 mg, 1.36 mmol) in anhydrous CH₃CN was added dropwise over 20 min a solution of (tBu)₂NOTA (432.3 mg, 1.21 mmol) in CH₃CN (10 mL) and K₂CO₃ (122.4 mg, 0.89 mmol). The reaction was stirred at room temperature for 72 h. Solvent was evaporated and the concentrate was diluted with water/DMF and purified by preparative RP-HPLC to yield a white solid (35.1 mg). HRMS (ESI) calculated for C₂₇H₄₃N₃O₆ (M+H)⁺506.3225; found 506.3234.

### 2-[7-but-3-ynyl-4-(carboxymethyl]-1,4,7-triazacyclononan-1-yl)acetic acid. NOTA-Butyne

To a solution of (tBu)₂NOTA (165.8 mg, 0.46 mmol) in 5 mL CH₃CN was added 4-bromo-1-butyne (44 μL, 62.3 mg, 0.47 mmol) and reaction mixture was stirred at room temperature for 72 h. Solvent was evaporated and the concentrate was purified by preparative

RP-HPLC to yield an oil. HRMS (ESI) calculated for C₂₂H₃₉N₃O₄ (M+H)⁺ 410.3013; found 410.3013. The purified product was acidified with 2 mL TFA and after 5 h diluted with water, frozen and lyophilized. HRMS (ESI) calculated for C₁₄H₂₃N₃O₆ (M+H)⁺ 298.1761; found 298.1757.

tert-butyl-2-(7-(4-aminobutyl)-4-{[(tert-butyl)oxycarbonyl]methyl}-1,4,7-triazacyclononan-1-yl)acetic acid. (tBu)₂NOTA-BA

To a solution of (tBu)₂NOTA (165.2 mg, 0.46 mmol) in 5 mL CH₃CN was added 4-(Boc-amino)butyl bromide (124.7 mg, 0.49 mmol), a pinch of K₂CO₃ and reaction mixture was stirred at room temperature for 72 h. Solvent was evaporated and the concentrate was treated with 1 mL CH₂Cl₂ and 0.5 mL TFA. After 5 min the solvents were evaporated and the crude oil was diluted with water/DMF and purified by preparative RP-HPLC to yield a white solid (137.2 mg, 69.3%). HRMS (ESI) calculated for C₂₂H₄₄N₄O₄ (M+H)⁺ 429.3435; found 429.3443.

### NOTA-BAEM: (BAEM =butyl amido ethyl maleimide)

To a solution of (tBu)₂NOTA-BM (29.3 mg, 0.068 mmol) in CH₂Cl₂ (3 mL) was added a 13-maleimido propionic acid NHS ester (16.7 mg, 0.063 mmol), 20 μL DIEA and stirred at room temperature overnight. Solvent was evaporated and the concentrate was acidified with 1 mL TFA. After 3 h, the reaction mixture was diluted with water and purified by preparative RP-HPLC to yield a white solid. HRMS (ESI) calculated for C₂₁H₃₃N₅O₇ (M+H)⁺ 468.2453; found 468.2441.

### 2-{4-[(4,7-bis-tert-butoxycarbonylmethyl)-[1,4,7]-triazacyclononan-1-yl)methyl]phenyl}acetic acid. (tBu)₂NOTA-MPAA

To a solution of 4-(bromomethyl)phenylacetic acid (593 mg, 2.59 mmol) in anhydrous CH₃CN (50 mL) at 0° C. were added dropwise over 1 h a solution of (tBu)₂NOTA (1008 mg, 2.82 mmol) in CH₃CN (50 mL). After 4 h, anhydrous K₂CO₃ (100.8 mg, 0.729 mmol) was added to the reaction mixture and allowed to stir at room temperature overnight. Solvent was evaporated and the crude was purified by preparative RP-HPLC (Method 5) to yield a white solid (713 mg, 54.5%). ¹H NMR (500 MHz, CDCl₃, 25° C., TMS) 6 1.45 (s, 18 H), 2.64-3.13 (m, 16 H), 3.67 (s, 2 H), 4.38 (s, 2 H), 7.31 (d, 2H), 7.46 (d, 2H); ¹³C (125.7 MHz, CDCl₃) δ 28.1, 41.0, 48.4, 50.9, 51.5, 57.0, 59.6, 82.3, 129.0, 130.4, 130.9, 136.8, 170.1, 173.3. HRMS (ESI) calculated for C₂₇H₄₃N₃O₆ (M+H)⁺ 506.3225; found 506.3210.

### 2-(4-(carboxymethyl)-7-{[4-(carboxymethyl)phenyl]methyl}-1,4,7-triazacyclononan-1-yl)acetic acid. NOTA-MPAA

To a solution of 4-(bromomethyl)phenylacetic acid (15.7 mg, 0.068 mmol) in anhydrous CH₃CN at 0° C. was added dropwise over 20 min a solution of (tBu)₂NOTA (26 mg, 0.073 mmol) in CH₃CN (5 mL). After 2 h, anhydrous K₂CO₃ (5 mg) was added to the reaction mixture and allowed to stir at room temperature overnight. Solvent was evaporated and the concentrate was acidified with 2 mL TFA. After 3 h, the reaction mixture was diluted with water and purified by preparative RP-HPLC to yield a white solid (11.8 mg, 43.7%). ¹H NMR (500 MHz, DMSO-d₆, 25° C.) δ 2.65-3.13 (m, 12 H), 3.32 (d, 2H), 3.47 (d, 2H), 3.61 (s, 2 H), 4.32 (s, 2 H), 7.33 (d, 2H), 7.46 (d, 2H); ¹³C (125.7 MHz, DMSO-d₆) 40.8, 47.2, 49.6, 50.7, 55.2, 58.1, 130.4, 130.5, 130.9, 136.6, 158.4, 158.7, 172.8, 172.9. HRMS (ESI) calculated for C₁₉H₂₇N₃O₆ (M+H)⁺ 394.1973; found 394.1979.

### (tBu)₂NOTA-MPAA NHS ester

To a solution of (tBu)₂NOTA-MPAA (175.7 mg, 0.347 mmol) in CH₂Cl₂ (5 mL) was added (1 M in CH₂Cl₂) DCC (347 μL, 0.347 mmol), N-hydroxysuccinimide (NHS) (42.5 mg, 0.392 mmol), and 20 μL N,N-diisopropylethylamine (DIEA). After 3 h, dicyclohexylurea (DCU) was filtered off and solvent evaporated. The crude product was purified by flash chromatography on (230-400 mesh) silica gel (CH₂Cl₂:MeOH (100:0 to 80:20) to yield the NHS ester (128.3 mg, 61.3%). HRMS (ESI) calculated for C₃₁H₄₆N₄O₈ (M+H)⁺ 603.3388; found 603.3395.

### NOTA-MPAEM: (MPAEM=methyl phenyl acetamido ethyl maleimide)

To a solution of (tBu)₂NOTA-MPAA NHS ester (128.3 mg, 0.213 mmol) in CH₂Cl₂ (5 mL) was added a solution of N-(2-aminoethyl) maleimide trifluoroacetate salt (52.6 mg, 0.207 mmol) in 250 μL DMF and 20 μL DIEA. After 3 h, the solvent was evaporated and the concentrate treated with 2 mL TFA. The crude product was diluted with water and purified by preparative RP-HPLC to yield a white solid (49.4 mg, 45%). HRMS (ESI) calculated for C₂₅H₃₃N₅O₇ (M+H)⁺ 516.2453; found 516.2452.

### tert-butyl-2-(7-(4-aminopropyl)-4-{[(tert-butyl)oxycarbonyl]methyl}-1,4,7-triazacyclononan-1-yl)acetic acid. (tBu)₂NOTA-PA

To a solution of (tBu)₂NOTA (391.3 mg, 1.09 mmol) in 5 mL CH₃CN was added Benzyl-3-bromo propyl carbamate (160 μL) and reaction mixture was stirred at room temperature for 28 h. Solvent was evaporated and the concentrate was dissolved in 40 mL 2-propanol, mixed with 128.7 mg of 10% Pd-C and placed under 43 psi H₂ overnight. The product was then filtered and the filtrate concentrated.The crude product was diluted with water/DMF and purified by preparative RP-HPLC to yield a white solid (353 mg). HRMS (ESI) calculated for C₂₁H₄₂N₄O₄ (M+H)⁺ 415.3291; found 415.3279.

### NOTA-PAEM: (PAEM =propyl amido ethyl maleimide)

To a solution of (tBu)₂NOTA-PM (109.2 mg, 0.263 mmol) in CH₂Cl₂ (3 mL) was added a β-maleimido propionic acid NHS ester (63.6 mg, 0.239 mmol), 20 μL DIEA and stirred at room temperature overnight. Solvent was evaporated and the concentrate was acidified with 1 mL TFA. After 3 h, the reaction mixture was diluted with water and purified by preparative RP-HPLC to yield a white solid (79 mg). HRMS (ESI) calculated for C₂₀H₃₁N₅O₇ (M+H)⁺ 454.2319; found 454.2296.

### ¹⁸F-Labeling of Functionalized Triazacyclononane Ligands

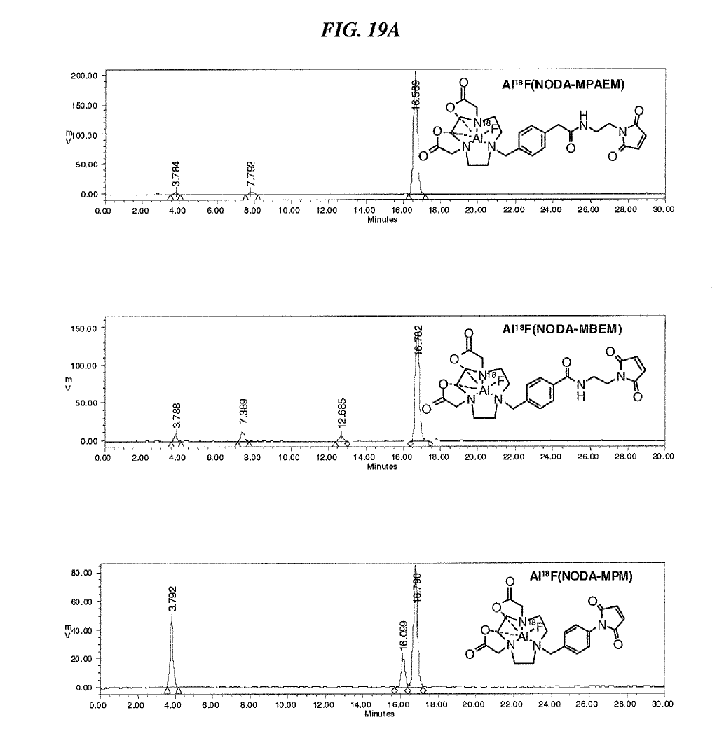

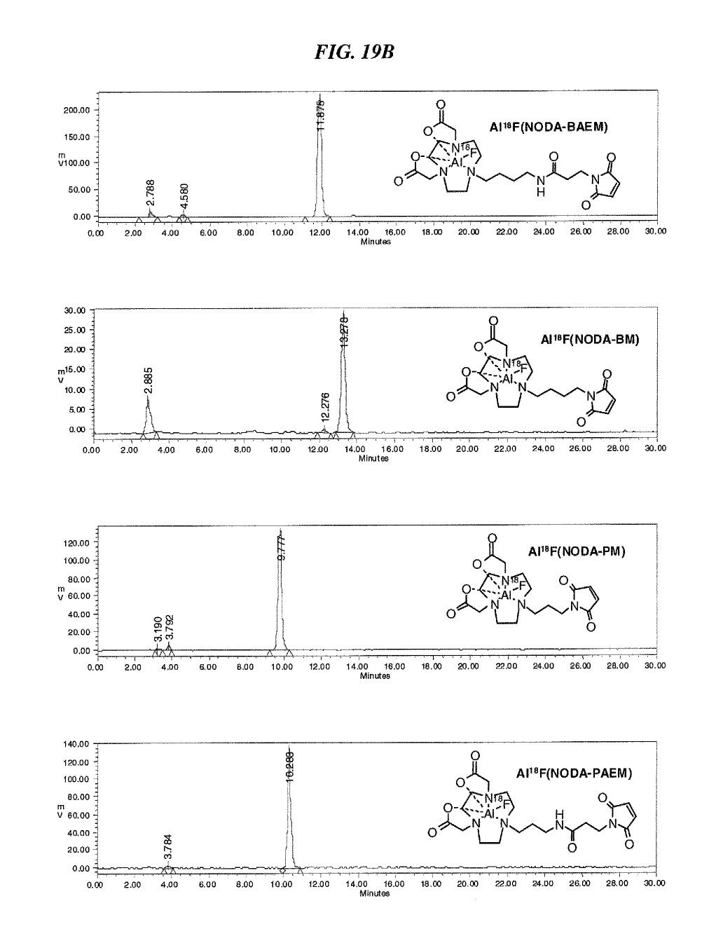

The functionalized triazacyclononane ligand (20 nmol; 10 μL), dissolved in 2 mM sodium acetate (pH 4), was mixed with AlCl₃ (5 μL, of 2 mM solution in 2 mM acetate buffer, 25-200 μL of ¹⁸F⁻ in saline, and 25-200 μL of ethanol. After heating at 90-105° C. for 15-20 min, 800 μL of deionized (DI) water was added to the reaction solution, and the entire contents removed to a vial (dilution vial) containing 1 mL of deionized (DI) water. The reaction vial was washed with 2×1 mL DI water and added to the dilution vial. The crude product was then passed through a 1-mL HLB column, which was washed with 2×1 mL fractions of DI water. The labeled product was eluted from the column using 3×200 μL of 1:1 EtOH/water. Radiochromatograms of the ¹⁸F-labeling of functionalized TACN ligands are shown in FIG. 19.

### ¹⁸F-labeling of NOTA-MPAEM

To 10 μL (20 nmol) 2 mM NOTA-MPAEM solution was added 5 μL 2 mM AlCl₃, 200 μL ¹⁸F⁻ solution [15.94 mCi, Na¹⁸F, PETNET] followed by 200 μL CH₃CN and heated to 110° C. for 15 minutes. The crude reaction mixture was purified by transferring the resultant solution into a Oasis® HLB 1 cc (30 mg) cartridge (P# 186001879, L# 099A30222A) and eluting with DI H₂O to remove unbound ¹⁸F⁻ followed by 1:1 EtOH/H₂O to elute the ¹⁸F-labeled peptide. The crude reaction solution was pulled through the HLB cartridge into a 10 mL vial and the cartridge washed with 6×1 mL fractions of DI H₂O (4.34 mCi). The HLB cartridge was then placed on a new 3 mL vial and eluted with 4×150 μL 1:1 EtOH/H₂O to collect the labeled peptide (7.53 mCi). The reaction vessel retained 165.1 μCi, while the cartridge retained 270 μCi of activity. 7.53 mCi [symbol] 61.2% of Al[¹⁸F]NOTA-MPAEM.

### ¹⁸F-labeling of NOTA-MPAEM

To 10 μL (20 nmol) 2 mM NOTA-MPAEM solution was added 5 μL 2 mM AlCl₃, 200 μL ¹⁸F⁻ solution [15.94 mCi, Na¹⁸F, PETNET] followed by 200 μL CH₃CN and heated to 110° C. for 15 minutes. The crude reaction mixture was purified by transferring the resultant solution into a Oasis® HLB 1 cc (30 mg) cartridge (P# 186001879, L# 099A30222A) and eluting with DI H₂O to remove unbound ¹⁸F⁻ followed by 1:1 EtOH/H₂O to elute the ¹⁸F-labeled peptide. The crude reaction solution was pulled through the HLB cartridge into a 10 mL vial and the cartridge washed with 6×1 mL fractions of DI H₂O (4.34 mCi). The HLB cartridge was then placed on a new 3 mL vial and eluted with 4×150 μL 1:1 EtOH/H₂O to collect the labeled peptide (7.53 mCi). The reaction vessel retained 165.1 μCi, while the cartridge retained 270 μCi of activity. 7.53 mCi [symbol] 61.2% of Al[¹⁸F]NOTA-MPAEM.

TABLE 21
¹⁸F-labeling of 20 nmol NOTA-MPAEM + 10 nmol Al³⁺

| Activity^{a} Na¹⁸F | Aqueous | CH₃CN | Isolated activity^{b} | RCY^{c} |
|---|---|---|---|---|
| (mCi) | (μL) | (μL) | (mCi) | (%) |
| 2.02 | 80 | — | 1.09 | 64.1 |
| 1.86 | 40 | 40 | 1.43 | 91.0 |
| 1.96 | 50 | 50 | 1.371 | 90.8 |
| 3.26 | 200 | 200 | 2.05 | 76.0 |
| 15.08 | 200 | 200 | 8.24 | 70.3 |
| 15.94 | 200 | 200 | 7.53 | 61.2 |

^{a}10 μL NOTA-MPAEM, 5 μL Al³⁺, 105-110° C., 15 min.
^{b}Isolated activity in (1:1) EtOH/H₂O after HLB column purification (SPE).
^{c}decay corrected RCY - based on synthesis time of 27-42 minutes.

### Conjugation of hMN14-Fab′-SH with Al¹⁸F(NOTA-MPAEM)

To the vial containing hMN14-Fab′-SH (1 mg, ˜20 nmoles) L # 112310 was added 200 μL PBS, pH 7.38 and 600 μL of the HLB purified Al¹⁸F(NOTA-MPAEM) (EtOH:H₂O::1:1). The crude reaction mixture was passed through a sephadex (G-50/80, 0.1 M NaOAc, pH 6.5) 3 mL spin column. The activity in the eluate was 4.27 mCi, while 1.676 mCi was retained on the spin column and 0.178 mCi in the empty reaction vial. 4.27 mCi [symbol] 68.6% of [Al¹⁸F]-hMN14-Fab.

To 800 μL of PBS was added 1 μL of eluate [symbol] injected 40 μL (SEC-HPLC) at 2.08 μm. Major product at 10.312 min.

### Serum Stability:

In an autosampler vial 200 μL of fresh human serum+50 μL of eluate [symbol] 149.6 μCi at 2.45 p.m. Incubated at 37° C. [symbol] [Al¹⁸F]-hMN14-Fab in serum.

To 4 μL of [Al¹⁸F]-hMN14-Fab in serum added 280 μL of buffer B (PBS)→injected 40 μL (SEC-HPLC) at 3.51 μm. Major product at 10.331 min.

### Immunoreactivity of ¹⁸F-hMN14-Fab:

To 100 μg carcinoembryonic antigen (CEA) [L # 2371505, Scripp's Labs] was added 200 μL 1% HSA in PBS, pH 7.38+100 μL PBS, pH 7.38→CEA in PBS. Added 4 μL of [Al¹⁸F]-hMN14-Fab in serum at 37° C. to 150 μL CEA in PBS→injected 40 μL (SEC-HPLC) at 4.33 p.m. Major product at 7.208 min.

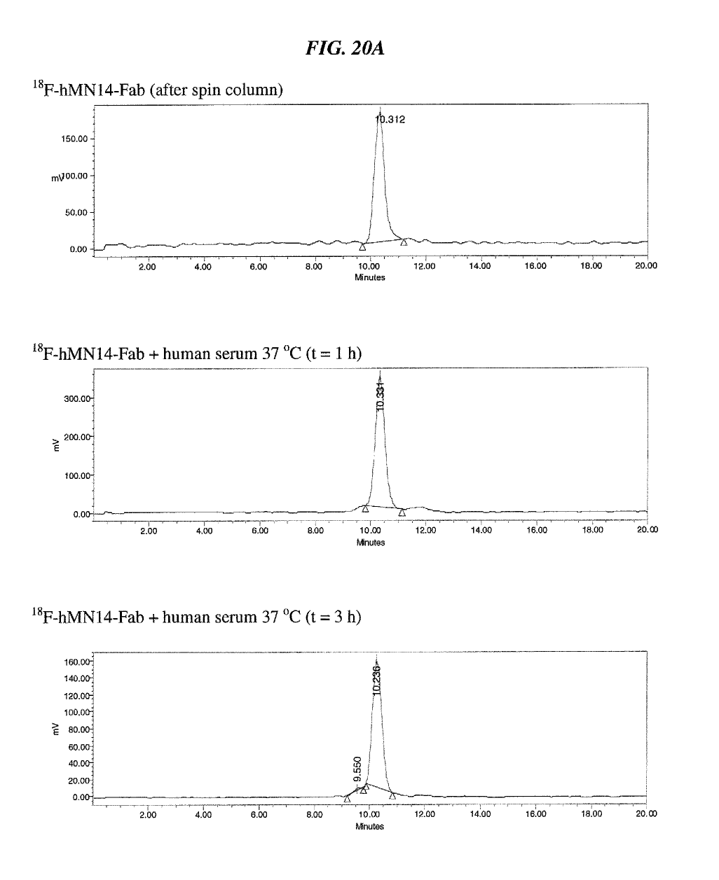

Radiochromatograms of spin column purified [Al¹⁸F]-hMN14-Fab, stability of [Al¹⁸F]-hMN14-Fab in human serum and its immunoreactivity with CEA are shown in FIG. 20.

Exemplary synthetic schemes for the bifunctional chelators are shown below.

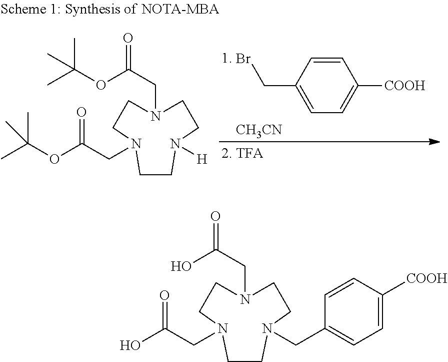

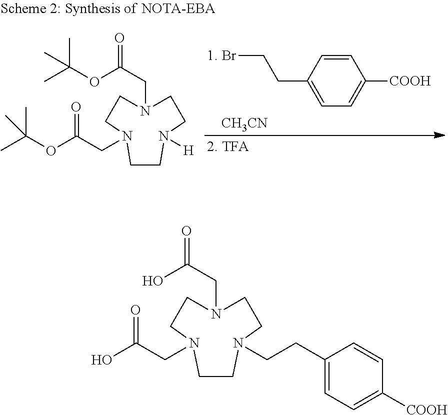

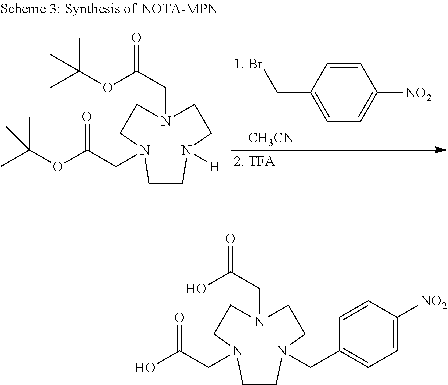

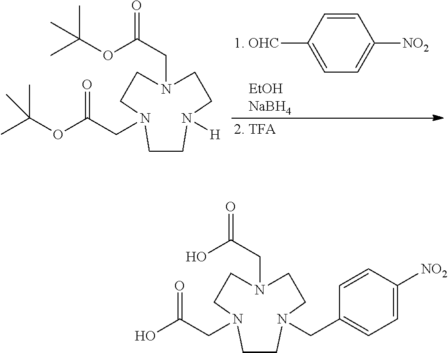

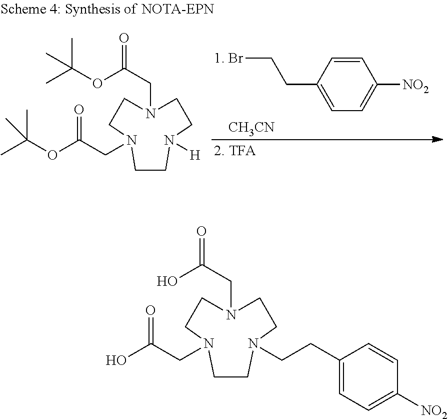

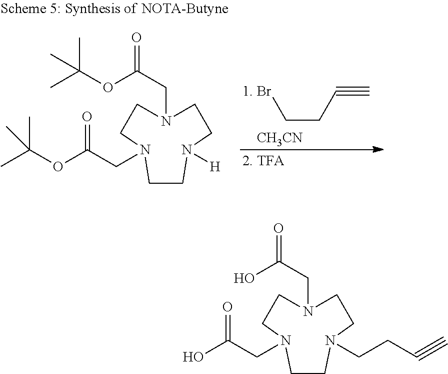

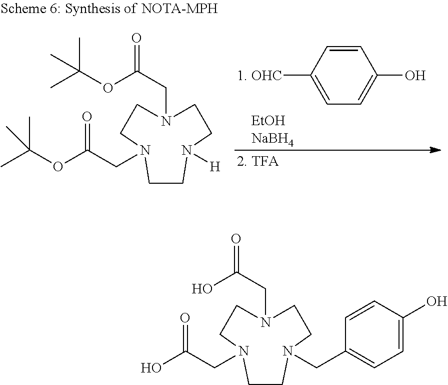

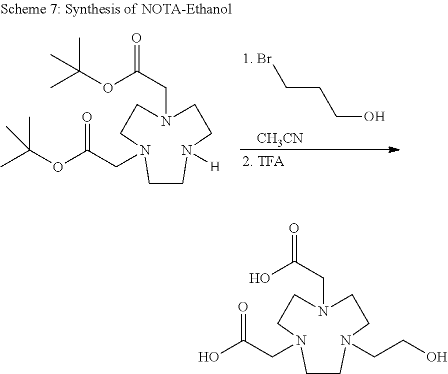

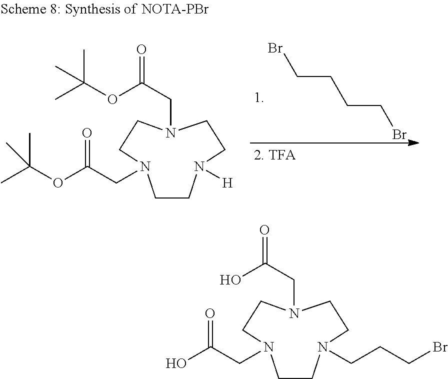 [SMILES: CC(C)(C)OC(=O)CN1CCNCCN(CC(=O)OC(C)(C)C)CC1 ; BrCCCCBr ; O=C(O)CN1CCN(CCCBr)CCN(CC(=O)O)CC1]

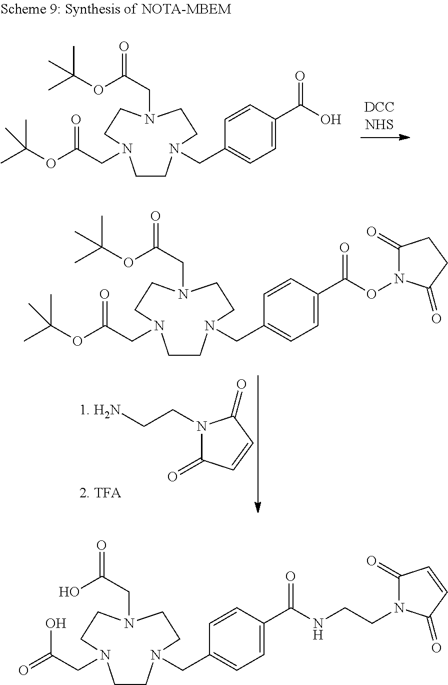

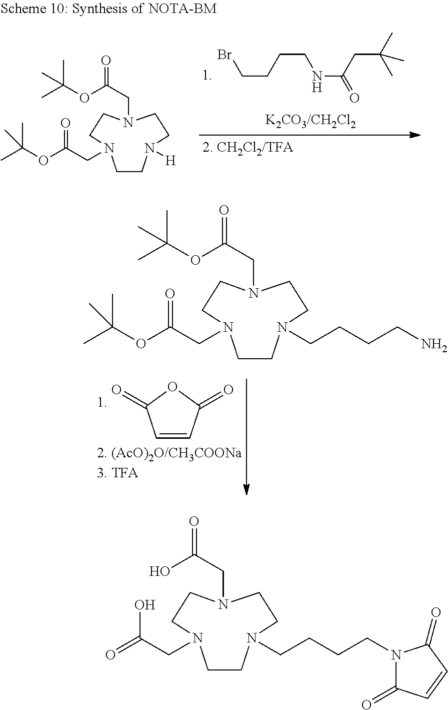

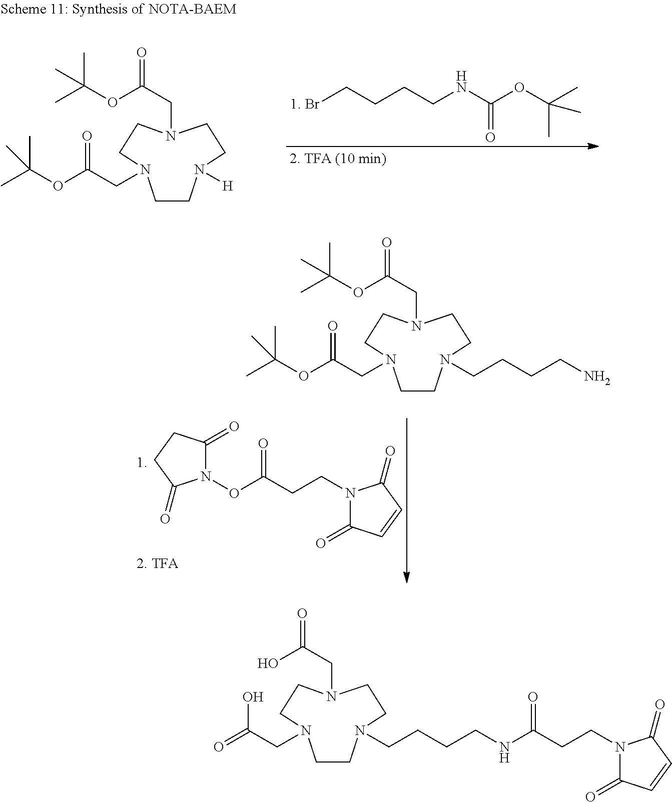

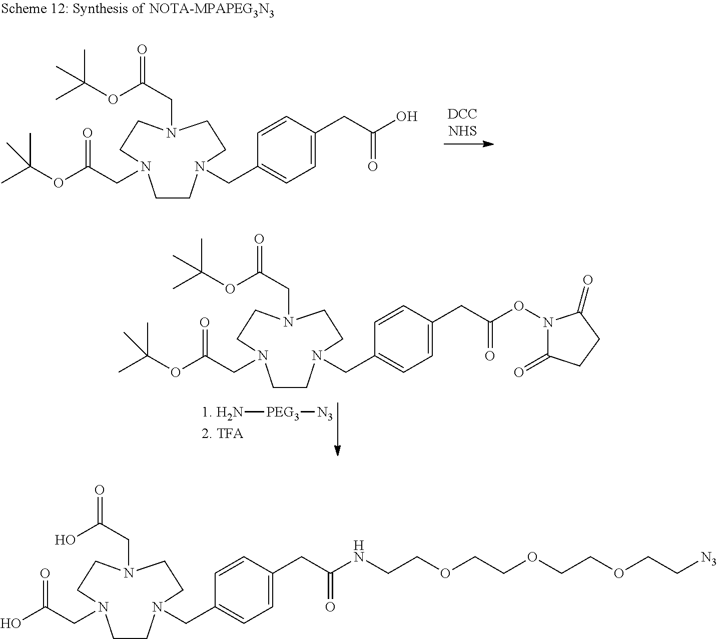

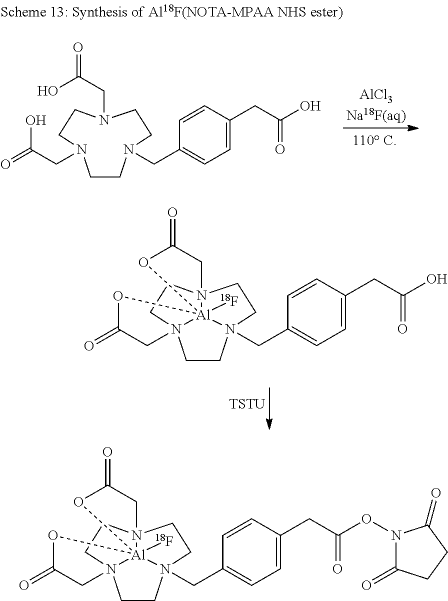

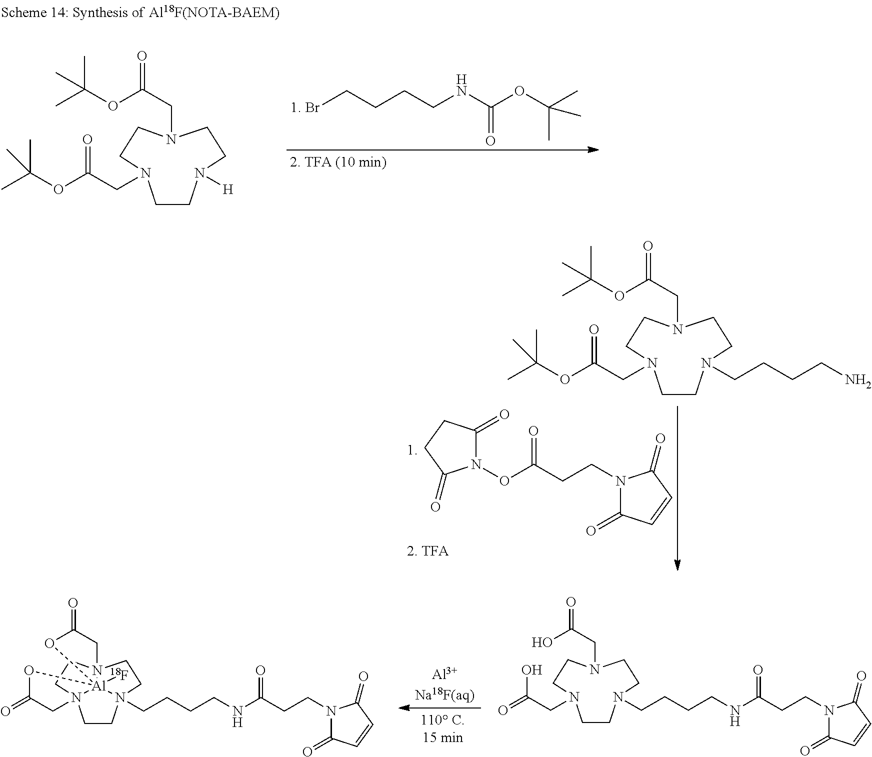

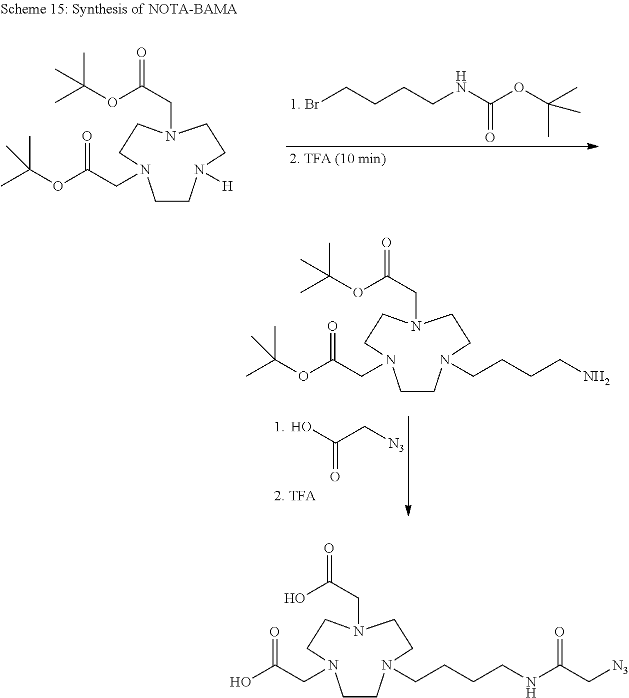

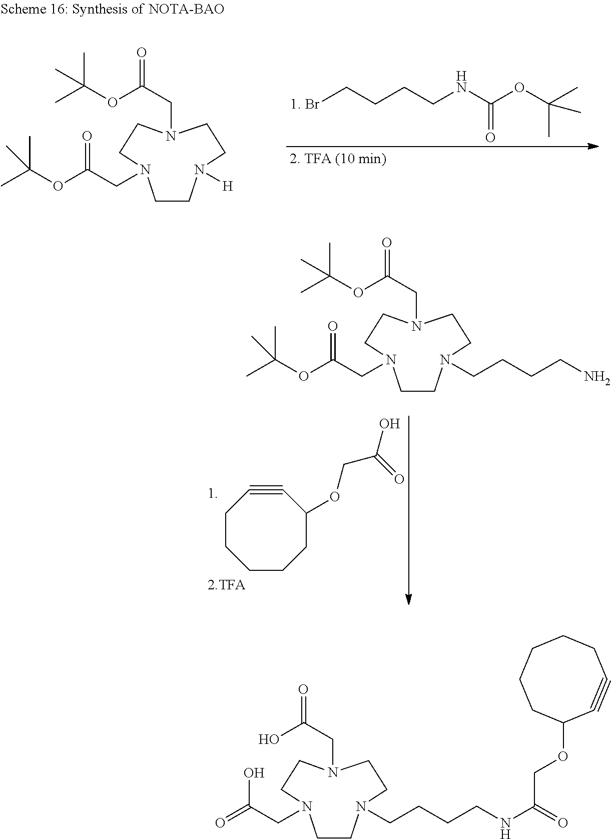

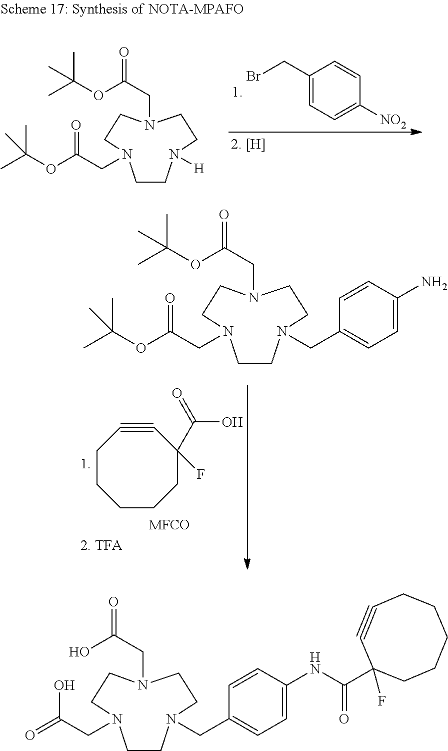

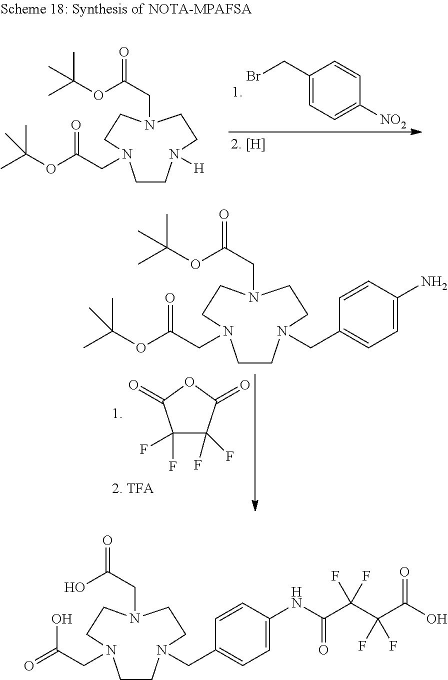

Exemplary structures of ¹⁸F-labeled probes are shown below.

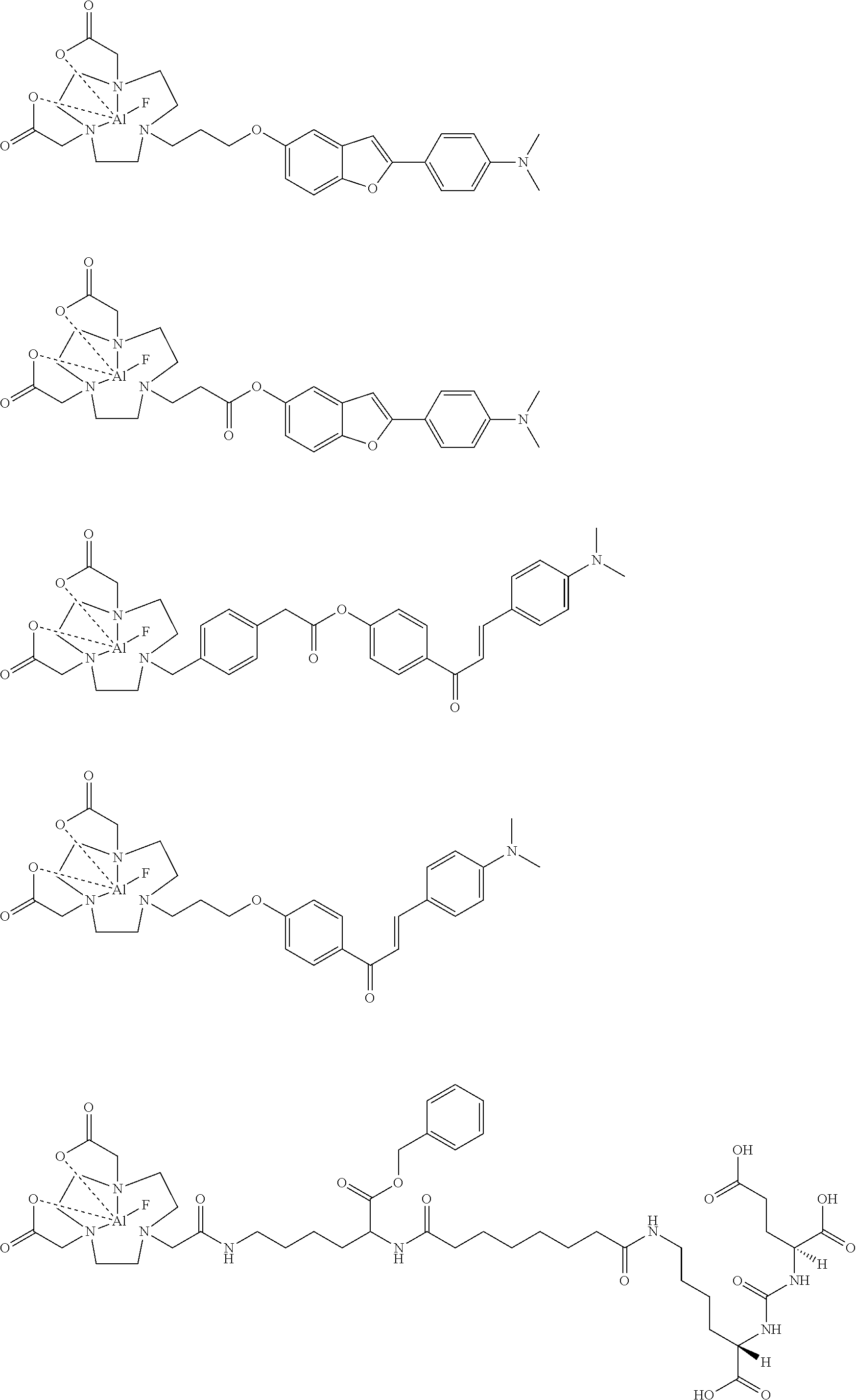

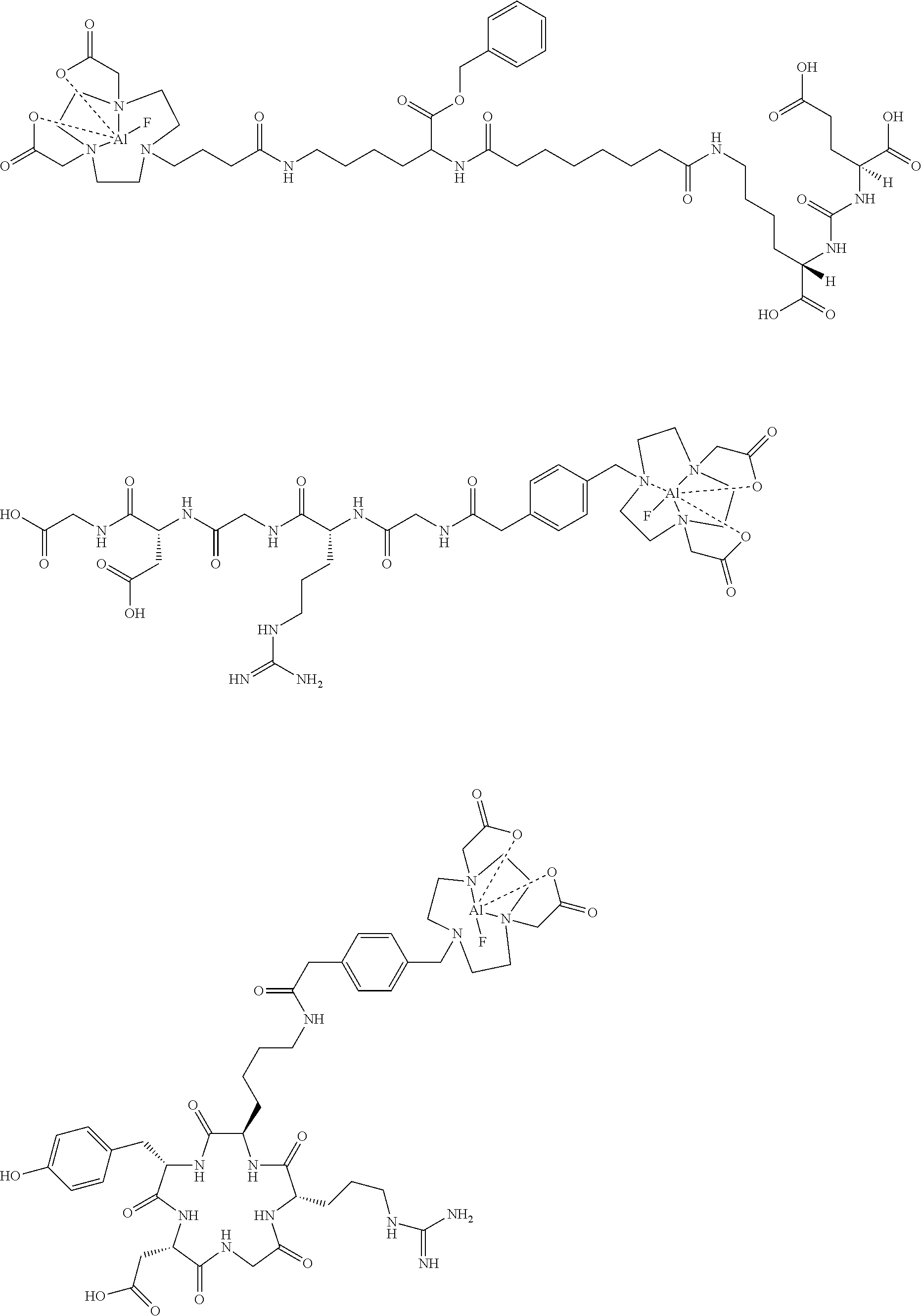

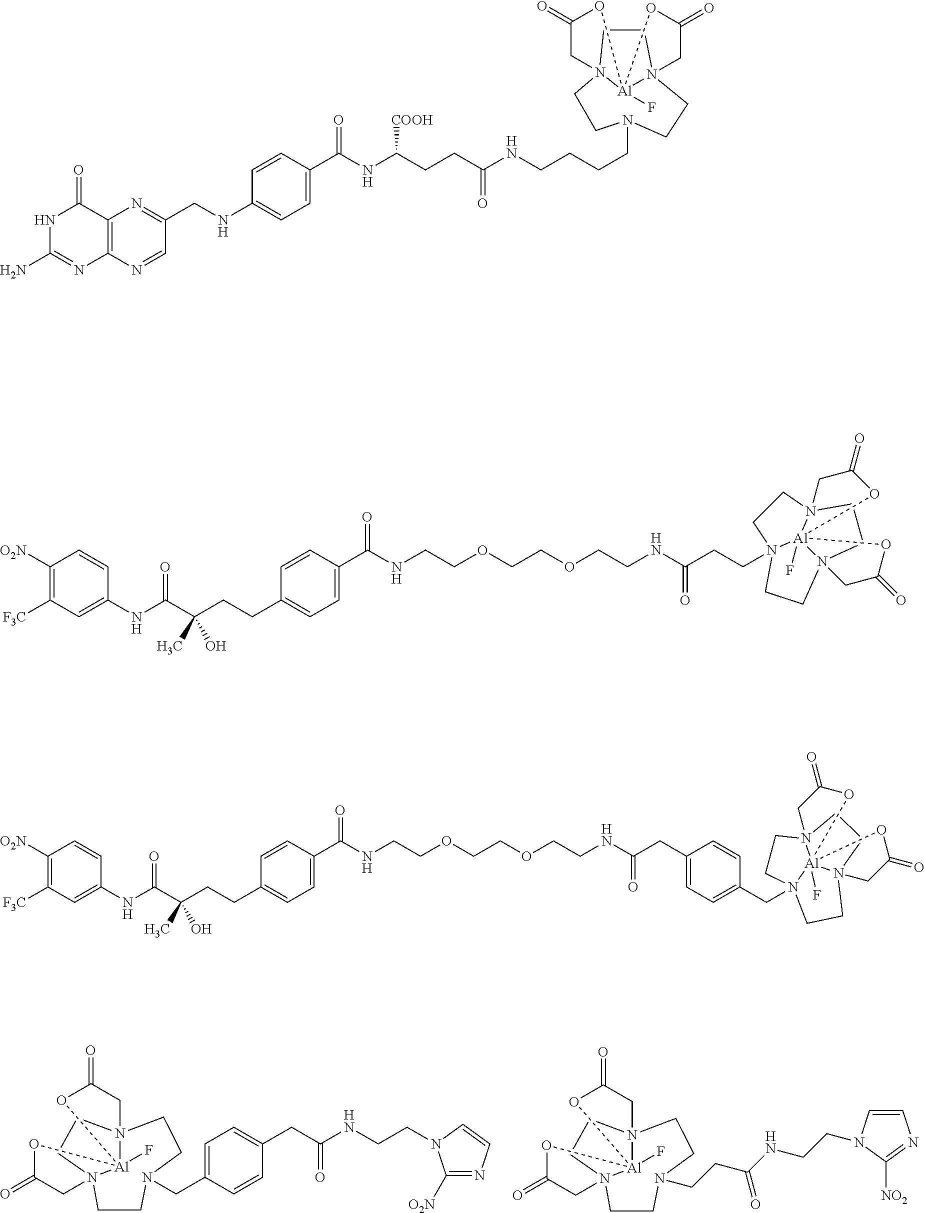

Conclusions

We have found that a novel class of triazacyclononane (TACN) derived BFCs, possessing a functionality that provides for an easy linkage onto biomolecules via solid phase or in solution, form stable complexes with a variety of metals. These BFCs also form remarkably stable Al¹⁸F chelates. Most ¹⁸F-labeling methods are tedious to perform, require the efforts of a specialized chemist, involve multiple purifications of the intermediates, anhydrous conditions, and generally end up with low RCYs. An advantage of this new class of BFCs is that they can be radiofluorinated rapidly in one step with high specific activity in an aqueous medium.

### Example 30

### Further Optimization of Kit Formulation

The effect of varying buffer composition on labeling efficiency was determined. Kits were formulated with 20 nmol IMP485 and 10 nmol AlCl₃.6H₂O in 5% α,α-trehalose. The buffers and ascorbic acid were varied in the different formulations. The peptide and trehalose were dissolved in DI water and the AlCl₃.6H₂O was dissolved in the buffer tested.

MES Buffer—4-morpholineethanesulfonic acid (MES, Sigma M8250), 0.3901 g (0.002 mol) was dissolved in 250 mL of DI H₂O and adjusted to pH 4.06 with acetic acid (8 mM buffer).

KHP Buffer—Potassium biphthalate (KHP, Baker 2958-1), 0.4087 g (0.002 mol) was dissolved in 250 mL DI H₂O pH 4.11 (8 mM buffer).

HEPES Buffer—N-2 hydroxyethylpiperazine-N′-2-ethane-sulfonic acid (HEPES, Calbiochem 391338) 0.4785 g (0.002 mol) dissolved in 250 mL DI H₂O and adjusted to pH 4.13 with AcOH (8 mM buffer).

HOAc Buffer—Acetic acid (HOAc, Baker 9522-02), 0.0305 g (0.0005 mol) was dissolved in 250 mL DI H₂O and adjusted to pH 4.03 with NaOH (2 mM buffer).

The AlCl₃.6H₂O (Aldrich 23078) was dissolved in the buffers to obtain a 2 mM solution of Al³⁺ in 2 mM buffer. IMP 485 0.0011 g (MW 1311.67, 8.39×10⁻⁷ mol) was dissolved in 419 μL DI H₂O. Ascorbic acid, 0.1007 g (Aldrich 25,556-4, 5.72×10⁻⁴ mol) was dissolved in 20 mL DI H₂O.

A variety of kits (summarized in Table 22) were prepared and adjusted to the proper pH by the addition of NaOH or HOAc as needed. The solution was then dispensed in 1 mL aliquots into 4, 3 mL lyophilization vials, frozen on dry ice and lyophilized. The initial shelf temperature for the lyophilization was −10° C. The samples were placed under vacuum and the shelf temperature was increased to 0° C. The samples were lyophilized for 15 hr and the shelf temperature was increased to 20° C. for 1 h before the vials were sealed under vacuum and removed from the lyophilizer. The kits were prepared with different buffers, at different pH values, with or without ascorbic acid and with or without acetate. After lyophilization, the kits were dissolved in 400 μL of saline and the pH was measured with a calibrated pH meter with a micro pH probe.

Radiolabeling—The kits were all labeled with ¹⁸F⁻ in saline (200 μL, PETNET) with ethanol (200 μL) and heated to ˜105° C. for 15 min. The labeled peptides were diluted with 0.6 mL DI H₂O and then added to a dilution vial containing 2 mL DI H₂O. The reaction vial was washed with 2×1 mL portions of DI H₂O, which were added to the dilution vial. The diluted solution was filtered through a 1 mL (30 mg) HLB cartridge (1 mL at a time) and washed with 2 mL DI H₂O. The cartridge was moved to an empty vial and eluted with 3×200 μL 1:1 EtOH/DI H₂O. The Al[¹⁸F]IMP485 was in the 1:1 EtOH/DI H₂O fractions. The isolated yield was determined by counting the activity in the reaction vial, the dilution vial, the HLB cartridge, the DI H₂O column wash and the 1:1 EtOH/DI H₂O wash adding up the total and then dividing the amount in the 1:1 EtOH/DI H₂O fraction by the total and multiplying by 100.

### Results

The results of the studies are shown in Table 22. All the labeling in the presence of 0.1 mg of ascorbic acid went well. The ascorbic acid appears to serve as a significant non-volatile buffer that keeps the pH the same before and after lyophilization (kits 1-4). When ascorbic acid is not used (kits 5-8) the pH can change significantly along with the radiolabeling yield. The KHP buffer, kit 8, was the best kit in the second batch. Higher levels of ascorbate might also stabilize the Al¹⁸F complex in solution and act as a transfer ligand for Al¹⁸F. The KHP buffer might also act as a transfer ligand for Al¹⁸F so the amount of KHP was increased from 5×10⁻⁷ mol/kit for kit 8 to 6×10⁻⁶ mol/kit for kit 11. The increase in KHP stabilized the pH better than kit 8 and gave a much better labeling yield. The kits with KHP+ascorbate (kit 12) and KHP +MES (kit 13) had slightly higher labeling yields. It may be that the higher levels of KHP and ascorbate act both as buffers and as transfer ligands to increase the labeling yields with those excipients. Citric acid is not a good buffer for [Al¹⁸F]-labeling (kit 14), it gives low labeling yields even when only 50 μL of 2 mM citrate was used in the presence 0.1 mg of ascorbate.

Increasing amounts of KHP, 0.1 M and above (kits 16-18) lead to lower labeling yields with more activity found in the aqueous wash from the HLB column.

TABLE 22
Results of labeling and pH studies

| | | pH before | pH after | |
|---|---|---|---|---|
| | | lyophili- | lyophili- | Isolated |
| Kit/lot Buffer | | zation | zation | % yield |
| 1. | HEPES + ascorbic | 4.07 | 3.83 | 82.6 |
| 2. | MES + ascorbic | 4.08 | 3.99 | 83.5 |
| 3. | NaOAc + ascorbic | 4.10 | 4.10 | 83.5 |
| 4. | KHP + ascorbic | 4.12 | 4.10 | 86.1 |
| 5. | HEPES No ascorbic + HOAc | 4.10 | 4.24 | 45.4 |
| 6. | MES No ascorbic + HOAc | 4.07 | 4.25 | 66.4 |
| 7. | HOAc No ascorbic + HOAc | 4.11 | 4.50 | 35.2 |
| 8. | KHP No ascorbic + HOAc | 4.01 | 4.09 | 71 |
| 9. | HEPES No ascorbic or NaOAc | 4.07 | 4.44 | 69 |
| 10. | MES No ascorbic or NaOAc | 4.06 | 4.62 | 75 |
| 11. | BM 20-57 KHP alone 0.015M | 4.07 | 4.05 | 83.9 |
| 12. | BM 20-57 KHP 0.015M + | 4.03 | 3.96 | 85.8 |
| | ascorbic | | | |
| 13. | BM 20-57 KHP 0.015M + | 4.10 | 4.09 | 87.0 |
| | MES 0.015M | | | |
| 14. | Citric acid | 4.03 | 3.93 | 31.2 |
| 15. | Ascorbic acid | 4.13 | 4.04 | 80.0 |
| 16. | KHP 0.1M | 4.09 | 3.82 | 80.7 |
| 17. | KHP 0.2M | 4.05 | 3.79 | 78.1 |
| 18. | KHP 0.4M | 4.02 | 3.79 | 69.0 |

It appears from these results that potassium biphthalate is an optimal buffer for labeling. The peptide labeling kits were therefore reformulated to utilize KHP in the labeling buffer. The reformulated kits gave very high isolated labeling yields of about 97% when 100 nmol of peptide was labeled in 1:1 ethanol/saline. The labeling and purification time was also simplified and reduced to 20 min. In addition to using the new buffering agent, potassium biphthalate (KHP), we also added more moles of buffer, which may help stabilize the pH during labeling. The peptide is purified through an Alumina N cartridge by adding more saline to the reaction after heating and pushing crude product through the cartridge. The unbound ¹⁸F⁻ and Al¹⁸F stick to the alumina and the labeled peptide is eluted very efficiently from the cartridge with saline. The formulation shown below is for a 20 nmol peptide kit but the same formulation is used for a 100 nmol peptide kit by adding more peptide and more Al³⁺ (60 nmol Al³⁺ for the 100 nmol peptide kit).

2 mM Al³⁺ in 2 mM KHP—Aluminum chloride hexahydrate, 0.0196 g (8.12×10⁻⁵ mol, Aldrich 23078, MW 241.43) was dissolved in 40.6 mL of 2 mM potassium biphthalate (KHP, JT Baker 2958-1, MW 204.23). This can be stored at room temperature for months.

Ascorbic Acid—Ascorbic acid, 0.100 g was dissolved in 20 mL DI H₂O. This is made fresh on the day of use.

5% Trehalose—α,α-Trehalose dihydrate, 2.001 g (J T Baker, 4226-04, MW 378.33) was dissolved in 20 mL DI H₂O. This can be stored at room temperature for weeks.

KHP Kit Buffer—KHP, 0.2253 g was dissolved in 18 mL DI H₂O (0.06 M). This solution can be kept for months at room temperature.

IMP485 solution—IMP485, 0.0049 g (3.74×10⁻⁶ mol, MW 1311.67) was dissolved in 1.494 mL DI H₂O (2.5×10⁻³ M). This solution can be stored for months at −20° C.

1M KOH—Potassium hydroxide (99.99% semiconductor grade, MW 56.11, Aldrich 306568) was dissolved in DI H₂O to make a 1 M solution.

Kit Formulation (20 nmol kit, 40 kits)—The peptide, IMP485 (320 μL, 8×10⁻⁷ mol) was placed in a 50 mL sterile polypropylene centrifuge tube (metal free) and mixed with 240 μL of the 2 mM Al³⁺ solution (4.8×10⁻⁷ mol) 800 μL of the ascorbic acid solution, 1600 μL of the 0.06 M KHP solution, 8 mL of the 5% trehalose solution and the mixture was diluted to 40 mL with DI H₂O. The solution was adjusted to pH 3.99-4.03 with a few microliters of 1 M KOH. The peptide solution was dispensed 1 mL/vial with a 1 mL pipette into 3 mL glass lyophilization vials (unwashed).

Lyophilization—The vials were frozen on dry ice, fitted with lyophilization stoppers and placed on a −20° C. shelf in the lyophilizer. The vacuum pump was turned on and the shelf temperature was raised to 0° C. after the vacuum was below 100 mtorr. The next morning the shelf temperature was raised to 20° C. for 4 hr before the samples were closed under vacuum and crimp sealed.

Radiolabeling—The ¹⁸F⁻ in saline was received from PETNET in 200 μL saline in a 0.5 mL tuberculin syringe. Ethanol, 200 μL, was pulled into the ¹⁸F⁻ solution and then the mixture was injected into a lyophilized kit containing the peptide. The solution was then heated in a 105° C. heating block for 15 min. Sterile saline, 0.6 mL was then added to the reaction vial and the solution was removed from the vial and pushed through an alumina N cartridge (SEP-PAK light, WAT023561, previously washed with 5 mL sterile saline) into a collection vial. The reaction vial was washed with 2×1 mL saline and the washes were pushed through the alumina column. The total labeling and purification time was about 20 min.

### Example 31

### Labeling at Reduced Temperature

The effect of varying the chelator structure on efficiency of labeling at reduced temperature was examined. A comparison of low temperature labeling of IMP466 (NOTA-Octreotide) with IMP485 showed that the simple NOTA ligand labels much better at low temperature than the NOTA-MPAA ligand.

In one embodiment, a temperature sensitive molecule, such as a protein, may be conjugated to multiple copies of a simple NOTA ligand. The protein can then be purified and formulated for Al¹⁸F-labeling (e.g., lyophilized). The protein kit was reconstituted with ¹⁸F⁻ in saline, heated for the appropriate length of time and purified by gel filtration or an alumina column. Tables 27 and 28 show the temperature effects of labeling IMP466 vs. IMP485.

TABLE 23
Temperature-dependent labeling for Al¹⁸F(IMP466)

| | % Yield | % Yield | % Yield | % Yield | % Yield |
|---|---|---|---|---|---|
| Temp ° C. | 25 μM | 50 μM | 100 μM | 250 μM | 500 μM |
| 50 | 3.3 | 8.6 | 14.5 | 20.5 | 37.1 |
| 70 | 21.3 | 47.4 | 58.0 | 82.8 | 93.6 |
| 90 | 29.0 | 50.7 | 70.3 | 83.0 | 93.5 |
| 100 | 34.3 | 55.8 | 77.4 | 84.0 | 94.5 |
| 110 | 34.9 | 60.3 | 78.1 | 87.9 | 90.5 |

TABLE 24
Temperature-dependent labeling for Al¹⁸F(IMP485)

| | % Yield | % Yield | % Yield | % Yield | % Yield |
|---|---|---|---|---|---|
| Temp ° C. | 25 μM | 50 μM | 100 μM | 250 μM | 500 μM |
| 50 | 1.31 | 3.29 | 3.18 | 6.10 | 12.99 |
| 70 | 7.01 | 12.8 | 22.90 | 36.8 | 39.8 |
| 90 | 22.2 | 38.3 | 82.3 | 85.4 | 85.3 |
| 100 | 48.6 | 76.1 | 91.8 | 94.6 | 96.6 |
| 110 | 61.6 | 74.4 | 96.4 | 94.0 | 96.8 |

The data show that by switching to a different chelating moiety, the efficiency of low temperature labeling with Al¹⁸F may be tripled at 50° C. Further modification of the chelating moiety may provide additional improvement of low temperature labeling. However, the 37% efficiency observed with IMP466 is sufficient to enable ¹⁸F PET imaging with temperature sensitive molecules if a sufficient number of chelating moieties are attached to the molecule.

We have also examined the effect of peptide concentration on low temperature labeling of IMP485. Kits were made with 10, 20, 40, 100 and 200 nmol of peptide and 0.6 equivalents of Al³⁺ respectively. The rest of the formulation was the same for all of the kits. The kits were labeled with 400 μL saline/EtOH and heated at 50-110° C. for 15 min and then purified through the Alumina N cartridge. The labeling results are reported as isolated yields in Table 25. At any temperature, increasing the concentration of peptide increased the efficiency of labeling. The results indicate that if the reaction volume can be decreased with the use of a microfluidics device then we can greatly reduce the amount of peptide and ¹⁸F⁻ needed to prepare a single dose of labeled peptide for PET imaging.

TABLE 25
Effect of peptide concentration on efficiency
of labeling as a function of temperature.

| | % Yield | % Yield | % Yield | % Yield | % Yield |
|---|---|---|---|---|---|
| Temp ° C. | 25 μM | 50 μM | 100 μM | 250 μM | 500 μM |
| 50 | 1.31 | 3.29 | 3.18 | 6.10 | 12.99 |
| 70 | 7.01 | 12.8 | 22.90 | 36.8 | 39.8 |
| 90 | 22.2 | 38.3 | 82.3 | 85.4 | 85.3 |
| 100 | 48.6 | 76.1 | 91.8 | 94.6 | 96.6 |
| 110 | 61.6 | 74.4 | 96.4 | 94.0 | 96.8 |

### Example 32

### Automated synthesis of ¹⁸ F-labeled molecules

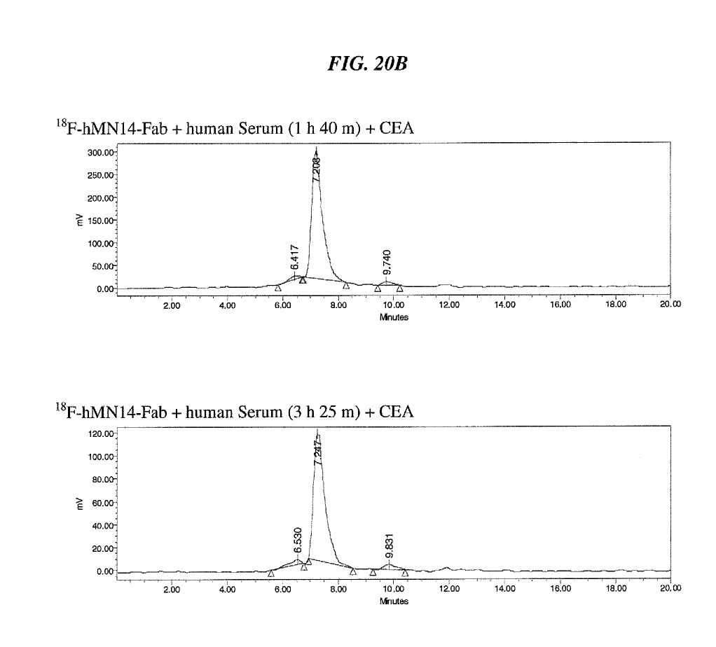

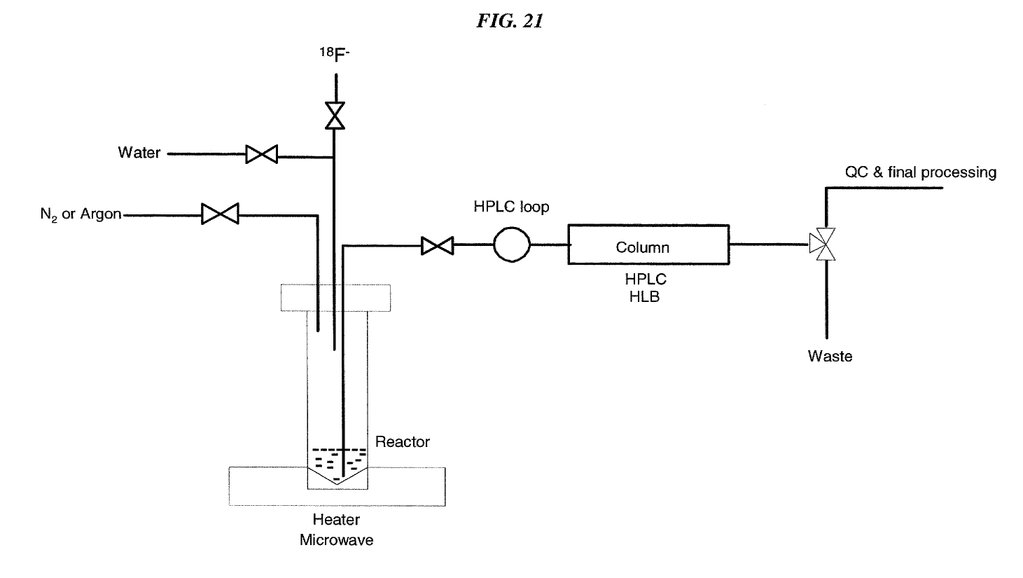

This Example compared the automated synthesis of ¹⁸F-FBEM published by Kiesewetter et al., (2011, Appl Radiat Isot 69:410-4) to that of Al¹⁸F(NOTA-MPAEM). The automated synthesis of ¹⁸F-FBEM was accomplished using a sophisticated synthesis module (see below), with a RCY of 17% in 95 min. Our synthesis module (FIG. 21) would include a heating device and a HLB cartridge or HPLC column. With NOTA-MPAEM we were able to get 67-79% RCY (decay corrected) in 40 min in one single step.

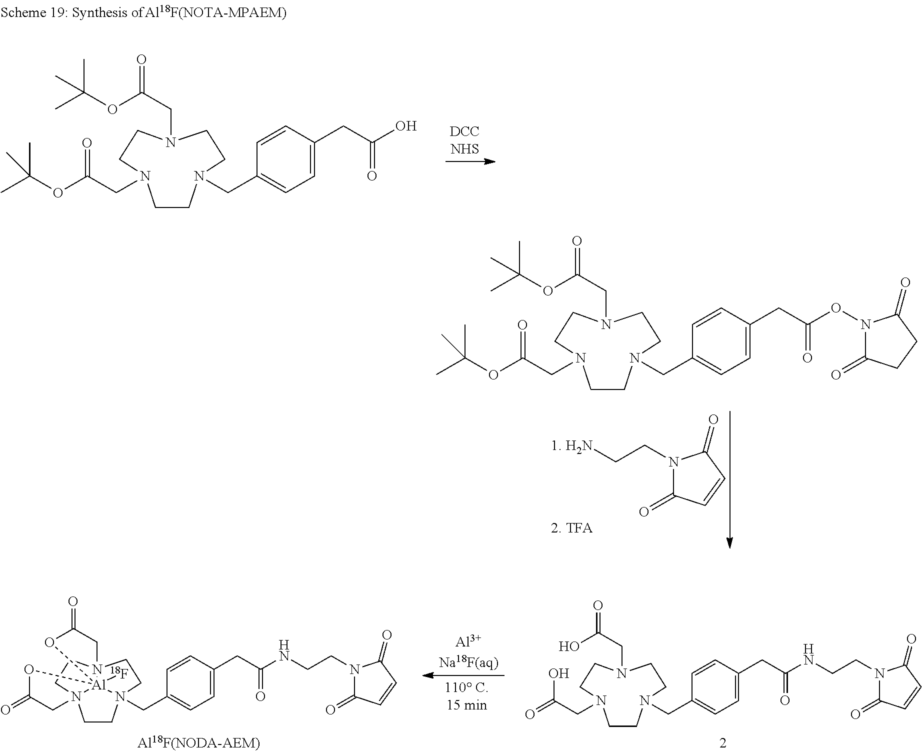

In both ¹⁸F-FBEM and ¹⁸F-FDG-MHO, the ¹⁸F is introduced first followed by a maleimide (Scheme 20 and 21). While NOTA-MPAEM—a maleimide containing BFC—is ¹⁸F-labeled in one final step (Scheme 19).

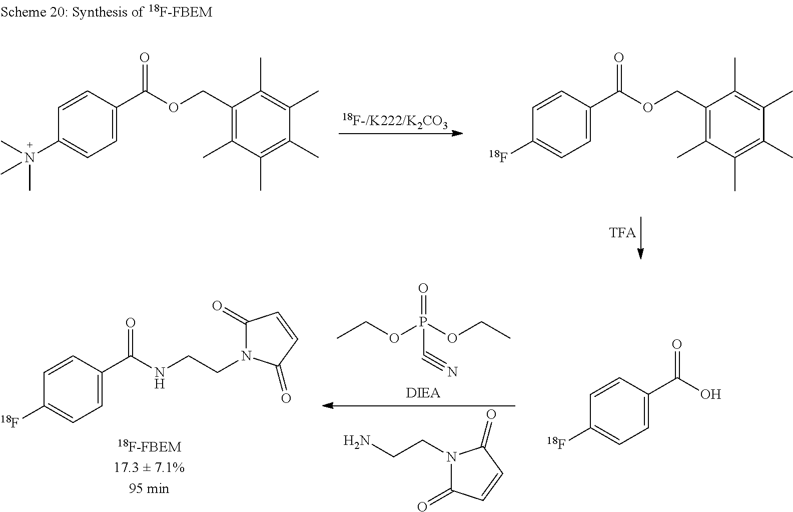

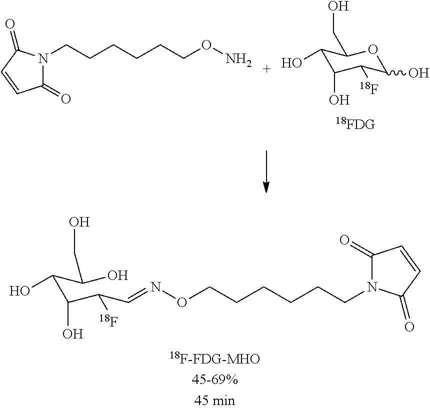 [SMILES: NOCCCCCCN1C(=O)C=CC1=O ; OCC1OC(O)C([18F])C(O)C1O ; O=C1C=CC(=O)N1CCCCCCO/N=C/C([18F])C(O)C(O)C(O)CO]

| Prosthetic | Synthesis | Synthesis | RCY | Specific |
|---|---|---|---|---|
| group | module | time | (%) | activity |
| ¹⁸F-SFB | TRACERlab ™ | 98 min | 44.3 ± 2.5 | 250-350 |
| 8-12 GBq | MX_{FDG} | | | GBq/μmol |
| (216-324 | | | | (6.8-9.5 |
| mCi) | | | | Ci/μmol) |

| Prosthetic | Synthesis | Synthesis | RCY* | Specific |
|---|---|---|---|---|
| group | module | time | (%) | activity |
| ¹⁸F-FBEM | Eckert & | 95 min | 17.3 ± 7.1 | 181-351 |
| | Ziegler | | | GBq/μmol |
| | | | | (4.9-9.5 |
| | | | | Ci/μmol) |

With 8.2 GBq (222 mCi) ¹⁸F⁻ provide 1.87 GBq (50.6 mCi) of ¹⁸F-FBEM in 96 min (22.8% uncorrected; 41.7% corrected for decay).
*not decay corrected.

### Example 33

### Room Temperature Labeling of Targeting Molecules Using Bifunctional Chelator (BFC) Moieties

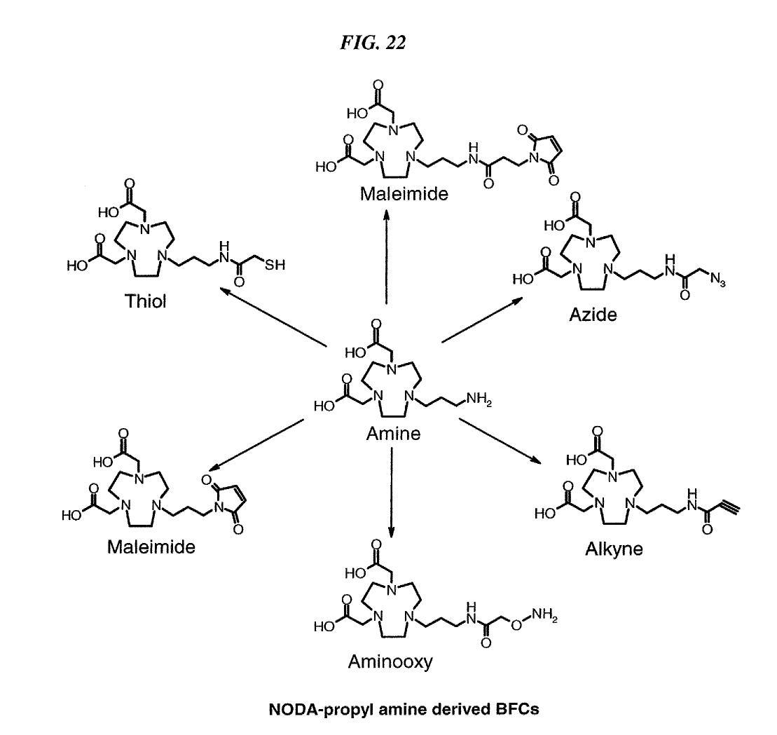

The objective of this Example was to perform ¹⁸F-labeling of temperature sensitive molecules at reduced temperatures, such as room temperature, with high radiochemical yield and high specific activity of the labeled molecule. Preferably, the labeling reaction is accomplished in 10 to 15 minutes in aqueous medium, with a total synthesis time of 30 minutes or less. More preferably, the labeling technique involves the initial reaction of a metal-¹⁸F or metal-¹⁹F with a bifunctional chelating (BFC) moiety at elevated temperature (e.g., 90 to 105° C.), followed by site-specific attachment of the BFC to the targeting molecule at a reduced temperature (e.g., room temperature). In certain embodiments, the BFC may be derived from the structure of NOTA-propyl amine (FIG. 22).

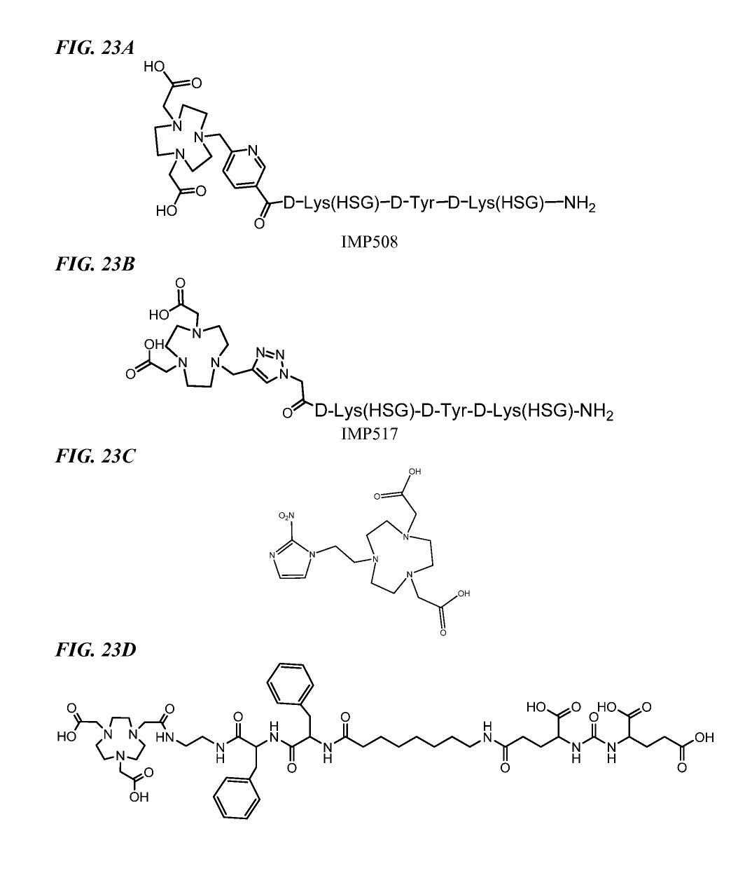

IMP508 (FIG. 23A) and IMP517 (FIG. 23B) were synthesized as disclosed below. The NOTA chelating moiety formed according to schemes 22 and 23 was attached to a bis-HSG peptide (IMP508), formulated into 20 nmol peptide kits and labeled with ¹⁸F.

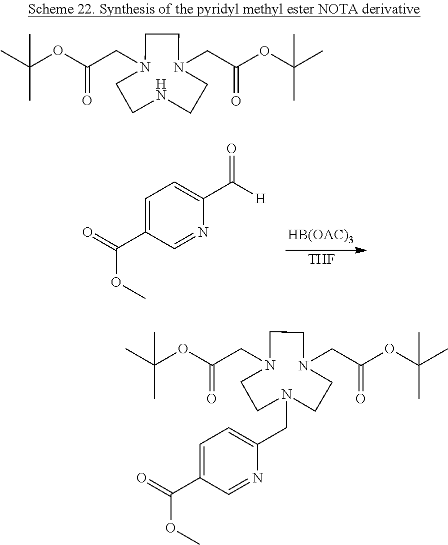

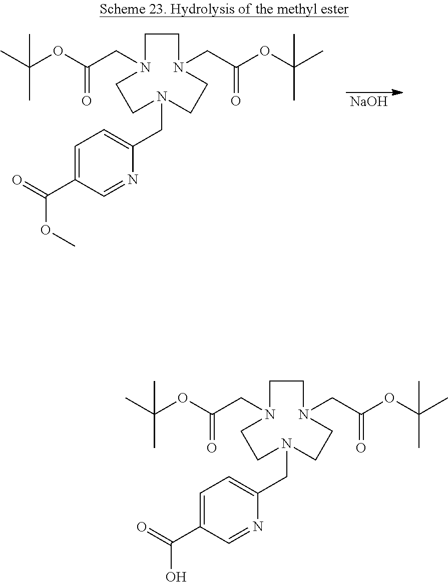

The methyl ester was synthesized as follows. The NO₂AtBu, 1.0033 g (2.807×10⁻³ mol) was mixed with 0.4638g (2.810×10⁻³ mol) of the methyl 6-formylnicotinate and dissolved in 10 mL THF. Triacetoxyborohydride, 0.6248 g (2.948×10⁻³ mol) was added and the reaction was stirred at room temperature for two days and an additional 0.3044 g of the borohydride was added. The reaction was quenched with H₂O after stirring 6.5 hr more at room temp. The product was extracted with dichloromethane, dried over Na₂SO₄, filtered and concentrated under reduced pressure to obtain the crude brown product. The product was purified by flash chromatography eluting with hexanes, 25% EtOAc/hexanes, 50% EtOAc/hexanes, 75% EtOAc/hexanes, 100% EtOAc, dichloromethane, 5% MeOH/94% dichloromethane/1% triethylamine and 10% MeOH/89% dichloromethane/1% triethylamine. The product, was isolated as a brown tar 0.455 g and was in the MeOH/ dichloromethane/ triethylamine fractions.

To synthesize the acid, the methyl ester (0.411 g, 8.12×10⁻⁴ mol) was dissolved in 5 mL dioxane and stirred with 0.8 mL of 1 M NaOH. The reaction was stirred for 18 hr at room temperature and another 1.3 mL of NaOH was added in portions as the reaction stirred at room temperature for another 8 hr. The reaction was quenched with 1 M citric acid and adjusted to pH 4.91 with 1 M NaOH. The product was extracted with dichloromethane. Some saturated NaC1 solution was added to the aqueous layer and the solution was again extracted with dichloromethane. The organic layers were combined, dried over Na₂SO₄, and concentrated to obtain 0.3421 g of the product (85% yield).

IMP517 was produced as disclosed in Scheme 24. The methyl ester triazole precursor was hydrolyzed and conjugated to the bis-HSG peptide to obtain IMP517 (FIG. 23B).

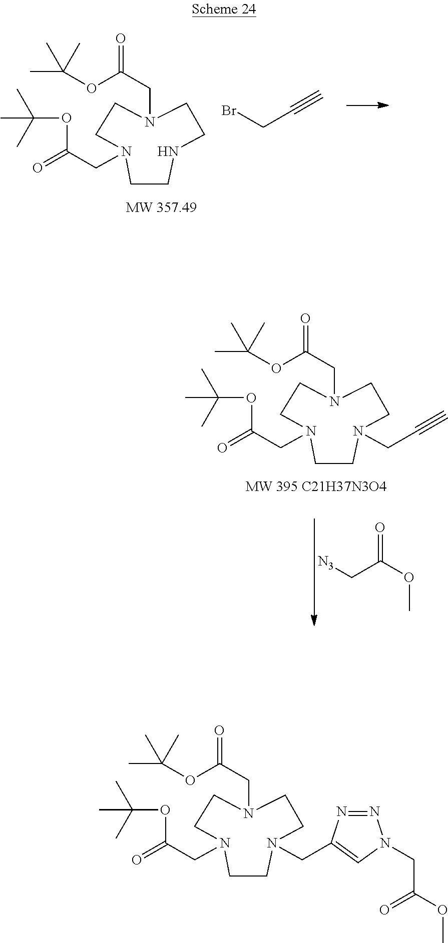

IMP517 was test labeled with different concentrations of peptide in 400 μL of saline. IMP485 was also labeled in 400 μL of saline for comparison.

| Peptide/nmol labeled in 400 μL | Isolated % |
|---|---|
| saline 110° C., 15 min | Yield |
| IMP517 2.5 nmol | 5.54 |
| IMP517 5 nmol | 31.1 |
| IMP517 10 nmol | 66.9 |
| IMP517 20 nmol | 85.8 |
| IMP485 20 nmol LSNE Kit | 78.3 |

IMP517 was labeled with F-18 in 400 μL of 1:1 EtOH/saline at different temperatures for 15 min.

| IMP517 (20 nmol) | Isolated % |
|---|---|
| Labeling temp. ° C. | Yield |
| 50 | 5.8 |
| 60 | 19.3 |
| 70 | 31.6 |
| 90 | 72.4 |
| 100 | 86.9 |
| 110 | 93.1 |

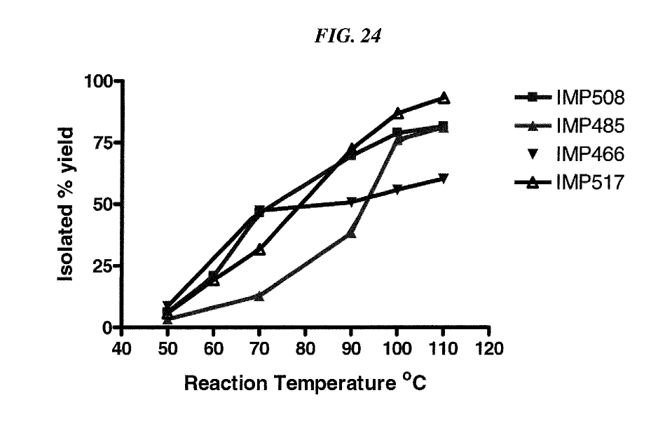

FIG. 24 compares the labeling of IMP517 20 nmol kits in 400 μL of 1:1 EtOH/saline heated for 15 min. IMP517 gave the highest labeling yields of the ligands tested so far and also gave high yields in saline alone. New NOTA derivatives with different functional groups in the vicinity of the 1,4,7-triazacyclononane ring were prepared and attached to a standard test peptide. The peptides were radiolabeled over a range of temperatures from 50 to 110° C. with and without a co-solvent. Two of these derivatives containing a pyridyl or a triazole group showed improved labeling yields at lower temperatures as well as labeling equal or better than the benzyl-NOTA standard at higher temperatures. Adding ethanol to the triazole derivative did not increase yields as much as the other derivatives, indicating that it may be possible to improve the radiolabeling yield at lower temperatures and reduce or eliminate the need for a co-solvent.

Alterations to the NOTA/NOTA ligand on a peptide can have a positive effect on the radiolabeling yield of the peptide, and may lead to ligands that can be used for direct one-step ¹⁸F labeling of some temperature-sensitive molecules.

### Example 34

### Non-Peptide, Small Molecule-Imaging Agents

A NOTA-2-nitroimidazole derivative (50 nmol, I mL) (FIG. 23C) used for hypoxia imaging was labeled in 0.1 M, pH 4, NaOAc buffer by mixing with 22.5 μL of 2 mM AlCl₃.6H₂O (45 nmol) in 0.1 M pH 4 NaOAc, and 50 μL of ¹⁸F⁻ in saline, then heating at 110° C. for 10 min to obtain the labeled complex in 85% yield. In vivo studies with the Al¹⁸F-NOTA-2-nitroimidazole showed the expected biodistribution and tumor targeting, with no evidence of product instability. The NOTA-DUPA-Pep molecule (FIG. 23D) was made for targeting the prostate-specific membrane antigen (PSMA). The ¹⁸F-labeled molecule was synthesized in 79% yield after HPLC purification to remove the unlabeled targeting agent.

### Example 35

### Large Peptide and Protein Labeling

NOTA-N-ethylmaleimide was attached to a cysteine side chain of the 40 amino acid exendin-4 peptide, which targets the glucagon-like peptide type-1 receptor (GLP-1 receptor) (Kiesewetter et al., 2012, Tharanostics 2:999-1009). The peptide was labeled with ¹⁸F⁻, using unpurified cyclotron target water to obtain the labeled peptide in 23.6±2.4% uncorrected yield in 35 min. The Al¹⁸F-labeled peptide had 15.7±1.4% ID/g in the tumor and 79.25±6.20% ID/g in the kidneys at 30 min, with low uptake in all other tissues.

The NOTA-affibody Z_{HER2 2395} (58-amino acid, 7 kDa) was labeled at 90° C. for 15 min with Al¹⁸F, the affibody, and acetonitrile (Heskamp et al., 2012, J Nucl Med 53:146-53). The labeling and purification process took about 30 min and the yield was 21±5.7%. Again, biodistribution studies supported the stability of the product with negligible bone uptake.

We also examined a two-step labeling method for temperature-sensitive molecules. The NOTA-MPAA ligand was attached to N-ethylmaleimide to make NOTA-MPAEM. The NOTA-MPAEM (20 nmol in 10 μL 2 mM, pH 4, NaOAc) was mixed with 5 μL 2 mM AlCl₃ in 2 mM, pH 4, NaOAc followed by 200 μL ¹⁸F⁻ in saline and 200 μL of acetonitrile. The solution was heated at 105-109° C. for 15 min and purified by SPE to produce the Al¹⁸F-NOTA-MPAEM in 80% yield. This product was then coupled to a pre-reduced antibody Fab’ fragment (20 nmol) by mixing the purified Al¹⁸F-NOTA-MPAEM at room temperature for 10 min, followed by isolation of the labeled Fab' by gel filtration. The labeled protein was obtained in an 80% yield. The total synthesis time for both steps combined was about 50 min, with an overall decay-corrected yield of about 50-60%.

### Example 36

### Residualization and In Vivo Clearance of Al¹⁸F Complexes

Lang et al. compared the biodistribution of ^{18F} on carbon, Al¹⁸F and ⁶⁸Ga attached to the same NOTA-PRGD2 peptide in the U-87 MG human glioblastoma model (Lang et al., 2011, Bioconjugate Chem 22:2415-22). They found that tumor uptake of the ¹⁸F-PPRGD2 peptide was 3.65±0.51% ID/g at 30 min PI compared to 1.85±0.30% ID/g at 2 h, indicating that the ¹⁸F activity was slowly clearing from the tumor between 30 min and 2 h (51% retention). The metal-complexed RGD peptides had higher tumor retention [4.20±0.23% ID/g (30 min), 3.53±0.45% ID/g (2 h) or 84% retention for Al¹⁸F-NOTA-PRGD2, and 3.25±0.62% ID/g (30 min), 2.66±0.32% ID/g (2 h), or 82% retention ⁶⁸Ga-NOTA-PRGD2] over the same period. These data show that the chelated AlF complex may be retained better in the tumor than the radiofluorinated compound with ¹⁸F bound to a carbon atom. The retention of activity also was seen with the exendin peptide and the affibody, where the activity cleared from the kidneys when the ¹⁸F was attached to a carbon atom (Kiesewetter et al., 2012, Eur J Nucl Med Mol Imaging 39:463-73; Kramer et al., 2008, Eur J Nucl Med Mol Imaging 35:1008-18), but was retained with the Al¹⁸F complex (Kiesewetter et al., 2012, Theranostics 2:999-1009; Heskamp et al., 2012, J Nucl Med 53:146-53). Retention of the radionuclide in a tissue could provide a targeting advantage, particularly in rapidly metabolizing tissues, such as damaged heart tissue.

### Example 37

### Labeling of NOTA-derivatized Octreotate

Exemplary targeting peptides of use in the claimed methods and compositions are disclosed below. The peptides are produced by standard synthesis techniques and conjugated to chelating moieties as disclosed in the Examples above. The NOTA-octreotate derivatives are labeled with Al-¹⁸F as described above and administered to patients with suspected neuroendocrine tumors. PET imaging is used to detect sst₂⁺ tumors by standard PET techniques, as disclosed above. The labeled targeting peptides provide high resolution images of both primary and metastatic tumors.

Labeling with other isotopic species, such as ⁶¹Cu, ⁶²Cu, ⁶⁴Cu, ⁶⁷Cu, ¹⁸F, ¹⁹F, ⁶⁶Ga, ⁶⁷Ga, ⁶⁸Ga, ⁷²Ga, ¹¹¹In, ¹⁷⁷Luu, ⁴⁴Sc, ⁴⁷Sc, ⁸⁶Y, ⁸⁸Y, ⁹⁰Y, ⁴⁵Ti and ⁸⁹Zr, show that the methods and compositions are not limited to Al-¹⁸F labeling, but rather are applicable to any radionuclide or other diagnostic agent that can bind to NOTA or a derivatized NOTA. Numerous exemplary species of derivatized NOTA are disclosed in the Examples above and any such chelating moiety may be utilized.

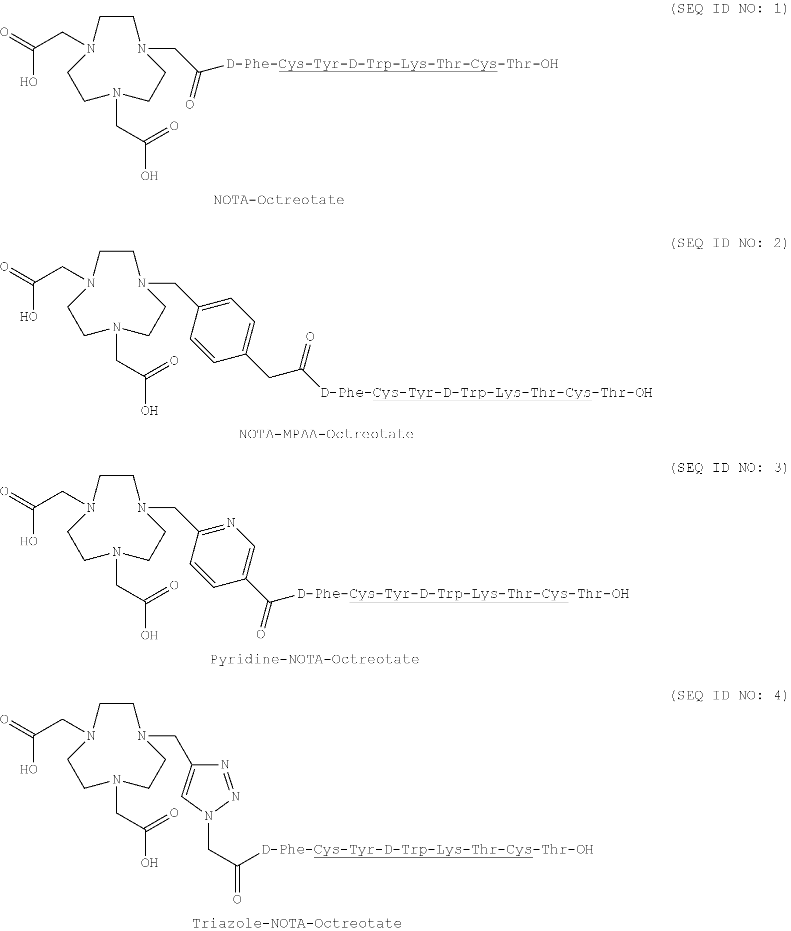

What is claimed is:

1. A compound comprising a chelating moiety and a peptide, wherein the structure of the compound is selected from the group consisting of:

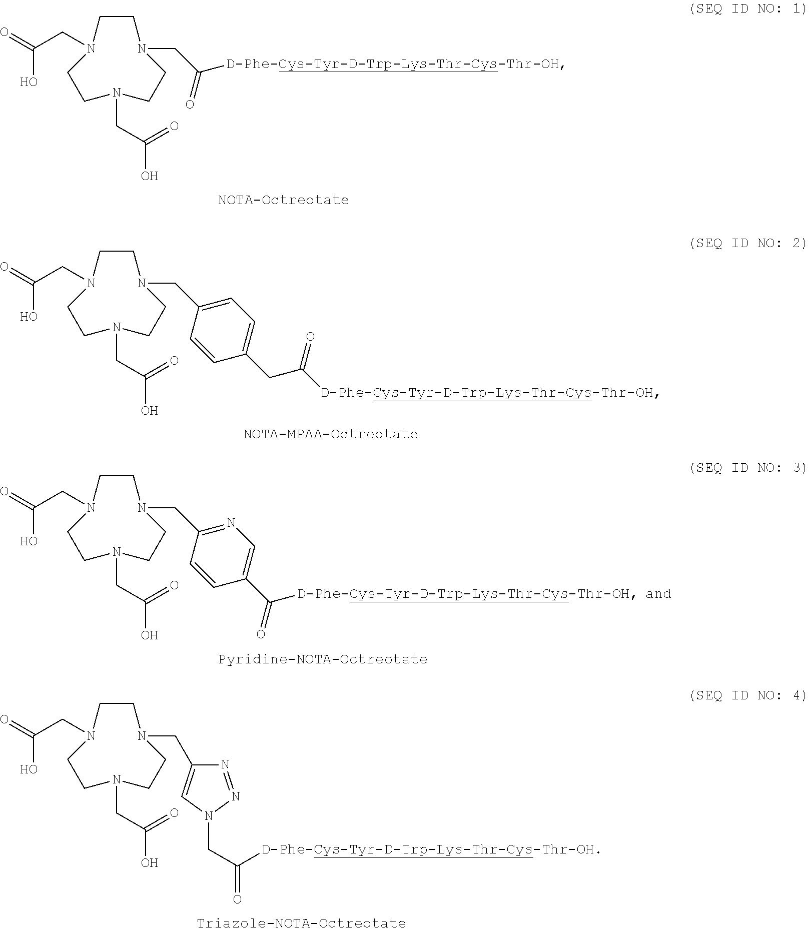

2. The compound of claim 1, further comprising a diagnostic agent or a therapeutic agent attached to the chelating moiety

3. The compound of claim 1, wherein the diagnostic agent is a metal-¹⁸F or metal-¹⁹F complex.

4. The compound of claim 3, wherein the metal is a group IIIA metal.

5. The compound of claim 4, wherein the metal is aluminum.

6. The compound of claim 2, wherein the diagnostic or therapeutic agent comprises an isotope selected from the group consisting of ⁶¹Cu, ⁶²Cu, ⁶⁴Cu, ⁶⁷Cu, ¹⁸F, ¹⁹F, ⁶⁶Ga, ⁶⁷Ga, ⁶⁸Ga, ⁷²Ga, ¹¹¹In, ¹⁷⁷Luu, ⁴⁴Sc, ⁴⁷Sc, ⁸⁶Y, ⁸⁸Y, ⁹⁰Y, ⁴⁵Ti and ⁸⁹Zr.

7. A composition comprising a compound according to claim 1.

8. The composition of claim 7, further comprising at least one component selected from the group consisting of water, an organic solvent, a buffer, phosphate, citrate, arginine, glutamine, sodium chloride, ascorbic acid, dextrose, maltose, sucrose, trehalose, sorbitol, mannitol, glycerol, albumin, a protamine, a detergent, and Tween 80.

9. The composition of claim 7, further comprising a diagnostic agent or a therapeutic agent attached to the chelating moiety

10. The composition of claim 9, wherein the diagnostic agent is a metal-¹⁸F or metal-¹⁹F complex.

11. The composition of claim 10, wherein the metal is a group IIIA metal.

12. The composition of claim 11, wherein the metal is aluminum.

13. The composition of claim 9, wherein the diagnostic or therapeutic agent comprises an isotope selected from the group consisting of ⁶¹Cu, ⁶²Cu, ⁶⁴Cu, ⁶⁷Cu, ¹⁸F, ¹⁹F, ⁶⁶Ga, ⁶⁷Ga, ⁶⁸Ga, ⁷²Ga, ¹¹¹In, ¹⁷⁷Luu, ⁴⁴Sc, ⁴⁷Sc, ⁸⁶Y, ⁸⁸Y, ⁹⁰Y, ⁴⁵Ti and ⁸⁹Zr.

14. A method of detecting, diagnosing and/or imaging an sst₂-expressing cancer comprising:
a) administering to a subject suspected of having an sst₂-expressing cancer a compound according to claim 1, wherein the compound is attached to at least one diagnostic agent; and
b) detecting or imaging the compound attached to the sst₂-expressing cancer.

15. The method of claim 14, wherein the diagnostic agent is a metal-¹⁸F or metal-¹⁹F complex.

16. The method of claim 15, wherein the metal is a group IIIA metal.

17. The method of claim 15, wherein the metal is aluminum.

18. The method of claim 14, wherein the diagnostic agent is selected from the group consisting of ⁶¹Cu, ⁶²Cu, ⁶⁴Cu, ¹⁸F, ¹⁹F, ⁶⁶Ga, ⁶⁷Ga, ⁶⁸Ga, ¹¹¹In, ¹⁷⁷Lu, ⁴⁴Sc, ⁴⁷Sc, ⁸⁶Y, ⁸⁸Y, ⁹⁰Y, ⁴⁵Ti and ⁸⁹Zr.

19. The method of claim 14, wherein the sst₂-expressing cancer is selected from the group consisting of neuroendocrine tumors (NET), gastroenteropancreatic NET, meningiomas, well-differentiated brain tumors, malignant lymphomas, renal cell carcinoma, breast carcinoma and lung carcinoma.

20. The method of claim 14, wherein the subjet is a human subject.

21. A method of treating an sst₂-expressing cancer comprising:
a) administering to a subject with an sst₂-expressing cancer a compound according to claim 1, wherein the compound is attached to at least one therapeutic isotope; and
b) delivering the therapeutic isotope to the cancer.

22. The method of claim 21, wherein the therapeutic isotope is selected from the group consisting of ⁶⁴Cu, ⁶⁷Cu, ⁶⁷Ga, ⁶⁸Ga, ⁷²Ga, ¹¹¹In, ¹⁷⁷Lu, ⁴⁴Sc, ⁴⁷Sc, ⁸⁶Y, ⁸⁸Y, ⁹⁰Y, ⁴⁵Ti and ⁸⁹Zr.

23. The method of claim 21, further comprising administering to the subject another therapeutic agent selected from the group consisting of cytotoxic agents, anti-angiogenic agents, pro-apoptotic agents, antibiotics, hormones, hormone antagonists, chemokines, drugs, prodrugs, toxins, enzymes, antibodies, antibody fragments, immunoconjugates, immunomodulators, oligonucleotides, siRNA, and RNAi.

24. The method of claim 23, wherein the therapeutic agent is selected from the group consisting of canertinib, dasatinib, erlotinib, gefitinib, imatinib, lapatinib, leflunomide, nilotinib, pazopanib, semaxinib, sorafenib, sunitinib, vatalanib, temsirolimus, rapamycin, ridaforolimus everolimus, ibrutinib, 5-fluorouracil, capecitabine, temozolomide, lambrolizumab, pidilizumab, ipilimumab and tremelimumab

25. The method of claim 23, wherein the drug is selected from the group consisting of 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celecoxib, chlorambucil, cisplatinum, Cox-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, doxorubicin, 2-pyrrolinodoxorubicine (2P-DOX), pro-2P-DOX, cyano-morpholino doxorubicin, doxorubicin glucuronide, epirubicin glucuronide, erlotinib, estramustine, epidophyllotoxin, erlotinib, entinostat, estrogen receptor binding agents, etoposide (VP 16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide (an aqueous form of DTIC), transplatinum, thalidomide, thioguanine, thiotepa, teniposide, topotecan, uracil mustard, vatalanib, vinorelbine, vinblastine, vincristine, vinca alkaloids and ZD1839.

26. The method of claim 23, wherein the toxin is selected from the group consisting of ricin, abrin, alpha toxin, saporin, ribonuclease (RNase), e.g., onconase, DNase I, Staphylococcal enterotoxin-A, pokeweed antiviral protein, gelonin, diphtheria toxin, Pseudomonas exotoxin, and Pseudomonas endotoxin.

27. The method of claim 23, wherein the immunomodulator is selected from the group consisting of a cytokine, a stem cell growth factor, a lymphotoxin, a hematopoietic factor, a colony stimulating factor (CSF), an interferon (IFN), erythropoietin, thrombopoietin, a tumor necrosis factor (TNF), granulocyte-colony stimulating factor (G-CSF), granulocyte macrophage-colony stimulating factor (GM-CSF), interferon-a, interferon-β, interferon-γ, interferon-λ, human growth hormone, N-methionyl human growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, tumor necrosis factor-a, tumor necrosis factor- B, mullerian-inhibiting substance, mouse gonadotropin-associated peptide, inhibin, activin, vascular endothelial growth factor, integrin, NGF-B, platelet-growth factor, TGF-α, TGF-β, insulin-like growth factor-I, insulin-like growth factor-II, macrophage-CSF (M-CSF), interleukin-1 (IL-1), IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, and lymphotoxin.

28. The method of claim 23, wherein the antibody, antibody fragment or immunoconjugate binds to an antigen selected from the group consisting of carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD4OL, CD45, CD46, CD52, CD54, CD55, CD59, CD64, CD66a-e, CD67, CD70, CD74, CD79a, CD80, CD83, CD95, CD126, CD133, CD138, CD147, CD154, CXCR4, CXCR7, CXCL12, HIF-1α, AFP, PSMA, CEACAM5, CEACAM-6, c-met, B7, ED-B of fibronectin, Factor H, FHL-1, Flt-3, folate receptor, GRO-β, HMGB-1, hypoxia inducible factor (HIF), HM1.24, insulin-like growth factor-1 (ILGF-1), IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, IP-10, MAGE, mCRP, MCP-1, MIP-1A, MIP-1B, MIF, MUC1, MUC2, MUC3, MUC4, MUC5, NCA-95, NCA-90, Ia, HM1.24, EGP-1, EGP-2, HLA-DR, tenascin, Le(y), RANTES, T101, TAC, Tn antigen, Thomson-Friedenreich antigens, tumor necrosis antigens, TNF-α, TRAIL receptor (R1 and R2), VEGFR, EGFR, PlGF, complement factors C3, C3a, C3b, C5a, C5, and an oncogene product.

29. The method of claim 23, wherein the antibody is selected from the group consisting of hR1 (anti-IGF-1R), hPAM4 (anti-pancreatic cancer mucin), hA20 (anti-CD20), hAl9 (anti-CD19), hIMMU31 (anti-AFP), hLL 1 (anti-CD74), hLL2 (anti-CD22), hMu-9 (anti-CSAp), hL243 (anti-HLA-DR), hMN-14 (anti-CEACAM5), hMN-15 (anti-CEACAM6), hRS7 (anti-EGP-1) and hMN-3 (anti-CEACAM6).

30. A method of detecting, diagnosing and/or imaging an sst₂-expressing cancer comprising:
a) administering to a subject suspected of having an sst₂-expressing cancer a compound comprising a chelating moiety conjugated to octreotate, wherein the chelating moiety is attached to a metal-18F or metal-19F complex; and
b) detecting or imaging the compound attached to the sst₂-expressing cancer by PET, SPECT or MRI.
